(19) United States
(12) Patent Application Publication
(10) Pub. No.: US 2015/0275276 A1
(43) Pub. Date: Oct. 1, 2015

(54) PCR REACTION MIXTURES AND METHODS OF USING SAME

(71) Applicants: SYNTEZZA MOLECULAR DETECTION ISRAEL LTD., Jerusalem (IL); MOLECULAR DETECTION INC., Wayne, PA (US)

(72) Inventors: Todd Wallach, Ambler, PA (US); Aryeh Gassel, Jerusalem (IL); David Wilson, East Sussex (GB); Tzvi Tzubery, Ofra (IL); Tal Salomon, Moshav Herev Leet (IL); Shira Glasner, Jerusalem (IL); Rina Maoz, Jerusalem (IL); Maoz Tal, Moshav Aviezer (IL); Sylvia Kachalsky, Gan Yavne (IL); Raphael Gassel, Jerusalem (IL)

(73) Assignee: MOLECULAR DETECTION ISRAEL LTD., JERUSALEM (IL)

(21) Appl. No.: 14/442,735

(22) PCT Filed: Nov. 15, 2013
(86) PCT No.: PCT/IL13/50949

§ 371 Date: May 14, 2015

Related U.S. Application Data
(60) Provisional application No. 61/726,630, filed on Nov. 15, 2012.

(51) Int. Cl. C12Q 1/68; G06F 19/20

(52) U.S. Cl. CPC C12Q 1/689; C12Q 1/686; G06F 19/20; C12Q 2600/16

(57) ABSTRACT

Provided herein are methods and compositions involving Polymerase Chain Reaction (PCR).

### FIELD

Provided herein are methods and compositions involving Polymerase Chain Reaction (PCR).

### BACKGROUND

Sepsis is a life-threatening illness in which toxic cytokines are released by the body in response to the presence of infectious bacteria or other pathogens. The worldwide annual incidence of sepsis is estimated to be 18 million cases. According to the US Center for Disease Control (CDC), the hospitalization rate of those with a principal diagnosis of septicemia or sepsis more than doubled from 2000 through 2008, to 24 per 10,000 population. Bloodstream infections (BSIs) are major causes of morbidity and mortality (Hall et al., 2011).

The true incidence of nosocomial BSIs is unknown, but it is estimated that about 250,000 cases occur annually in the USA. Some studies have reported the incidence of BSI to be around 1% in the intensive care unit (ICU) and 36% in bone marrow transplant recipients. The crude mortality rate has been reported to range from 12% in total hospital populations to 80% in ICU patients. The rate of mortality directly attributable to BSIs in ICU patients has been estimated to be 16-40%.

For patients with symptoms of septic shock, current guidelines recommend the administration of antibiotics within 1 hour after diagnosis, and within 3 hours for patients with earlier-stage sepsis symptoms. In the absence of microbiological information within this time frame, current practice relies on the empiric use of broad-spectrum antibiotics while the pathogen is cultured, identified and then subjected to antibiotic susceptibility testing over the course of several days. Culturing is required in order to “grow” pathogen to levels sufficient for detection, with the ability to distinguish true signal from noise, as current methods are not sensitive enough without culturing to detect pathogen in quantities less than 10-30 colony forming units per milliliter (cfu/ml).

Inadequate and/or delayed empirical antimicrobial therapy is the primary determinant of mortality, morbidity and increased hospital length of stay for sepsis patients. Mortality from sepsis increases at a rate of 8% for every hour that the patient is not receiving the antimicrobial therapy (Daniels 2011). Approximately 30-50% of all patients presenting with the clinical symptoms of sepsis receive inappropriate antimicrobial therapy for the first several days, because the causative pathogen and its antibiotic resistance profile is unknown at the time therapy is initiated. The use of inappropriate antibiotics is also discouraged because it increases the burden of antibiotic resistance in general.

Polymerase Chain Reaction (PCR) and Real-Time Quantitative PCR

The development of the polymerase chain reaction (PCR) made possible the in vitro amplification of nucleic acid sequences. PCR is described inter alia in U.S. Pat. No. 4,683,195; U.S. Pat. No. 4,683,202; and U.S. Pat. No. 4,965,188.

Additionally, commercial vendors, such as Applied Biosystems (Foster City, Calif.), market PCR reagents and publish PCR protocols.

PCR is designed to amplify a particular region of the target DNA known as the “amplicon”. PCR typically begun with an initial denaturation step, to enable efficient utilization of template in the first amplification cycle, followed by cycles of PCR amplification. In each cycle of PCR amplification, a double-stranded target sequence is denatured, primers are annealed to each strand of the denatured target, and the primers are extended by the action of a DNA polymerase, referred to as the “denaturation”, “annealing”, and “extension” steps. A final extension step may be included to fill in the protruding ends of newly synthesized PCR products. The specificity of amplification depends on the specificity of primer hybridization. Primers are selected to be complementary to, or substantially complementary to, sequences occurring at the 3′ end of each strand of the target nucleic acid sequence. Classically, product formation is analyzed after the conclusion of amplification, known as “endpoint PCR”.

Quantitative PCR, sometimes referred to as “real-time PCR” or “qPCR”, utilizes the same amplification scheme as PCR, with 2 oligonucleotide primers flanking the DNA segment to be amplified. In qPCR, the reaction products are monitored as they are formed. Several methods that rely on fluorescence at a specific wavelength can be used for real-time monitoring. One method used in real-time monitoring employs DNA-intercalating fluorescent dyes, such as SYBR® Green fluorescent dye (which also can be used in endpoint PCR). Another method adds a target-specific oligonucleotide probe that is labeled at 1 end with a fluorescent tag and at the other end with a fluorescent quencher (Molecular Beacon Probes), which separate from one another upon target binding, thus increasing fluorescence. In another variation, TaqMan® probes, the probes bind to the DNA target, and their fluorescent labels are cleaved from the probe during primer extension, thereby releasing the fluorescent tag.

As specialized type of endpoint PCR uses DNA-intercalating fluorescent dyes in combination with controlled melting, which is described inter alia in Won et al, Rapid identification of bacterial pathogens in positive blood culture bottles by use of a broad-based PCR assay coupled with high-resolution melt analysis. J Clin Microbiol 48:3410-3413; Yang et al, Rapid identification of biothreat and other clinically relevant bacterial species by use of universal PCR coupled with high-resolution melting analysis. J Clin Microbiol 47: 2252-2255; and US Pat. Appl. Publ. No. 2011/0045479 to Andreas Tobler, which is incorporated herein by reference, and the references cited in these publications.

Use of qPCR to Detect Polynucleotide Sequences of Interest in Clinical Specimens

qPCR has been used to detect target polynucleotide sequences of interest in test samples. One exemplary type of target polynucleotide sequences are those characteristic of pathogens of interest, typically assayed in clinical specimens to test for infectious disease. U.S. Pat. No. 6,664,080 to Klaus Pfeffer, entitled “TaqMan™-PCR for the detection of pathogenic E. coli strains”; US Pat. App. No. 2009/0181363, entitled “Non-Invasive Detection of Fish Viruses by Real-Time PCR”; US Pat. App. No. 2006/0177818, entitled “Method of detection of classical swine fever”, which are incorporated herein by reference, and references cited therein.

qPCR has also been used to specifically detect antibiotic-resistant pathogens such as methicillin-resistant Staphylococcus aureus (MRSA), where the mecA gene is integrated into the SA chromosome. For example, U.S. Pat. No. 8,017,337 to Yosef Paitan, entitled “Methods, Compositions and Kits for Detection and Analysis of Antibiotic-Resistant Bacteria”, which is incorporated herein by reference, describes use of multiplex qPCR that amplifies (a) a gene specific for the target bacteria; (b) an antibiotic-resistance gene; and (c) the bridging region between a known region of the bacterial genome and the usual site of integration of the antibiotic-resistance cassette.

Linear-after-the-exponential (LATE) PCR has also been used to amplify and detect polynucleotides, as described inter alia in Rice et al, Gentile et al, and US Pat. Appl. Publ. No. 2004/0053254 to Wangh et al, which is incorporated herein by reference, and the references cited therein. However, the disclosed methods failed to achieve a high sensitivity (low limit of detection), such as that needed for a sepsis assay in a clinical setting, and a relatively small number of primer sets and probes, such that such tests would be not be sufficient to detect the desired number of target polynucleotides for a sepsis test.

The detection of pathogen DNA and antibiotic-resistance polynucleotides in blood samples, in order to accurately and rapidly diagnose sepsis and determine appropriate antibiotic therapy, is quite challenging for a number of reasons: Firstly, a 10-ml. whole blood sample may contain as few as 10 copies of pathogen DNA, equivalent to at most 5 copies after extraction, using current extraction techniques. Second, a minimum of 1-2 copies of a DNA target of interest are needed to provide the level of reliability required. As a result, a sample containing 5 DNA copies can be divided into no more than 2 separate PCR reaction tubes (or reaction wells or chambers) to enable each tube to obtain at least 1-2 copies. Furthermore, meaningful coverage of clinically relevant antibiotic resistance genes and pathogen species to guide treatment of sepsis patients requires amplification of 12-30 different amplicons (6-15 primer pairs in each of 2 separate reactions) in order to achieve differential identification of 12-30 different DNA markers. The currently prior art multiplex qPCR methods do not enable this high level of activity.

Rapid detection of pathogen DNA and antibiotic-resistance polynucleotides in blood samples, in order to provide rapid and clinically relevant results, thus remains a vexing problem until today.

### SUMMARY

Those skilled in the art will appreciate, in light of the present disclosure, that identification of the presence of a particular pathogen and the presence or absence of particular antibiotic-resistance genes can help guide antibiotic therapy of a patient with a suspected infection.

The described methods and compositions are directed to improvement of existing PCR methods, for example regarding their ability to confirm a suspected case of sepsis and identify a variety of common (and in some embodiments, less common) pathogens and antibiotic-resistance genes present in the blood, even when present in very low copy number (approximately one copy per milliliter). The present inventors have developed methods and compositions for producing actionable results in just a few hours, instead of the 1-6 days typically required using culturing and antibiotic-resistance plating methods. In some embodiments, the highly-multiplexed design and high sensitivity of the assay enables detection of samples containing more than one pathogen and/or more than one antibiotic-resistance gene, even when present in very different amounts.

An additional aspect relates to kits for the in vitro amplification of nucleic acid sequences, for detection of pathogens and antibiotic-resistance polynucleotides, and for confirmation and diagnosis of a suspected case of sepsis, utilizing the described reaction mixtures.

Additionally, the inventors have also found that, when asymmetric PCR is performed with activatable primers such as ribo-primers and the like, which are cleaved as part of their activation process, both the pre-cleavage and post-cleavage melting temperatures play a role in determining the efficacy. The inventors have found particular combinations of these 2 parameters useful in enabling successful asymmetric PCR of GC-rich regions.

In jurisdictions allowing it, all patents, patent applications, and publications mentioned herein, both supra and infra, are incorporated herein by reference.

As used herein “comprising” is to be interpreted as specifying the presence of the stated features, integers, steps, or components as referred to, but does not preclude the presence or addition of one or more features, integers, steps, or components, or groups thereof. Thus, for example, a method comprising a given step may contain additional steps. Additionally, the term “comprising” is intended to include embodiments encompassed by the terms “consisting essentially of” and “consisting of.” Similarly, the term “consisting essentially of” is intended to include embodiments encompassed by the term “consisting of.”

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### BRIEF DESCRIPTION OF THE DRAWINGS

FIG. 1. Plots of performance of the VIM-PB 1 probe in monoplex and triplex reactions. A. Amplification curve. B. Melting curve. C. Plot of first derivative of fluorescence during melting, or Δf/ΔT; namely the first derivative of the decrease of fluorescence during melting, which describes the change in fluorescence as it depends on the change in temperature. For this and all Figures, the horizontal axis is cycle number (for amplification curves) or temperature in ° C. (for the other plots). The fluorescence in the amplification and melt panels is depicted in arbitrary units set by the apparatus.

FIG. 2. Plots of performance of VIM-PB2 in monoplex and triplex. A. Amplification curve. B. Melting curve. C. First derivative of fluorescence during melting.

FIG. 3. Plots of performance of NDM-PB1 in monoplex and triplex. A. Amplification curve. B. Melting curve. C. First derivative of melting fluorescence.

FIG. 4. Plots of performance of NDM-PB2 in monoplex and triplex. A. Amplification curve. B. Melting curve. C. First derivative of melting fluorescence.

FIG. 5. Plots of performance of 16SGN-PB in monoplex and triplex with VIM-PB1 and NDM-PB1. A. Amplification curve. B. Melting curve. C. First derivative of melting fluorescence.

FIG. 6. Plots of performance of 16SGN-PB in monoplex and triplex with VIM-PB2 and NDM-PB2. A. Amplification curve. B. Melting curve. C. First derivative of melting fluorescence.

FIG. 7. Superimposition of the curves of separate amplification of vim+16SGN, NDM+16SGN, and 16SGN, in each case in the presence of VIM-PB1, NDM-PB1, and 16SGN-PB. A. Amplification curve. B. Melting curve. C. First derivative of melting fluorescence.

FIG. 8. Superimposition of the curves of separate amplifications of vim+16SGN, NDM+16SGN, and 16SGN, in each case in the presence of VIM-PB2, NDM-PB2, and 16SGN-PB. A. Amplification curve. B. Melting curve. C. First derivative of melting fluorescence.

FIG. 9. Plots of monoplex performance of Spn9802-PB1 and Spn9802-PB2. A. Amplification curve. B. Melting curve. C. First derivative of melting fluorescence. For this Figure and FIGS. 10-12, samples were tested in triplicate, and the results between samples were consistent, as depicted by the different lines that closely track one another—in some cases too close together to be discriminated from one another.

FIG. 10. Plots of monoplex performance of IC-PB 1 and IC-PB2. A. Amplification curve. B. Melting curve. C. First derivative of melting fluorescence.

FIG. 11. Plots of monoplex performance of IC-PB 1 (A-C), IC-PB3 (D-F), and IC-PB4 (G-I), all plotted on the same scale, showing amplification curves (top), melting curves (middle), and first derivative of melting fluorescence (bottom). Top: Amplification curve. Middle: Melting curve. Bottom: First derivative of melting fluorescence.

FIG. 12. Plots of monoplex performance of tuf-PB1 (A-C), tuf-PB2 (D-F), and tuf-PB3 (G-I), all plotted on the same scale, showing amplification curves (top), melting curves (middle), and first derivative of melting fluorescence (bottom). Top: Amplification curve. Middle: Melting curve. Bottom: First derivative of melting fluorescence.

FIG. 13. Superimposition of the curves of separate amplifications of GES, OXA-48, and KPC, in each case in the presence of most of the GN tube primers. A. Amplification curve. B. Melting curve. C. First derivative of melting fluorescence.

FIG. 14. Superimposition of the curves of separate amplifications of vim, NDM, and 16S-GN, in each case in the presence of most of the GN tube primers. A. Amplification curve. B. Melting curve. C. First derivative of melting fluorescence.

FIG. 15. Plots of symmetric and asymmetric amplification of a GC-rich region of the KPC gene, using KPC-F2 and KPC-R2. A. Amplification curve. B. Melting curve. C. First derivative of melting fluorescence. Samples were tested in triplicate, and the results between samples were consistent, as depicted by the different lines that closely track one another.

FIG. 16. Plots of symmetric and asymmetric amplification of a GC-rich region of the NDM gene, using NDM-F2 and NDM-R2. A. Amplification curve. B. Melting curve. C. First derivative of melting fluorescence. Samples were tested in duplicate, and the results between samples were consistent, as depicted by the different lines that closely track one another.

### DETAILED DESCRIPTION

Provided herein are methods for detecting the presence of a pathogen and antibiotic-resistance polynucleotides; and compositions and kits that perform the methods.

Definitions

“Multiplex PCR” refers to a PCR wherein multiple sequences are simultaneously amplified in the same reaction mixture. Generally in such methods, distinct sets of primers are employed for each sequence being amplified. The described methods are believed to be applicable to be multiplex PCR and in other embodiments, also non-multiplex PCR. In certain embodiments, multiplex qPCR is utilized.

The term “strain” as used herein, refers to a subset of a pathogen species exhibiting an identifiable characteristic not present in members of the same species in general.

Reference herein to amplification of a polynucleotide is intended to encompass amplification of the entire sequence or a portion thereof.

Reference herein to a “set of primers” includes, in various embodiments, both instances where a single forward and single reverse primer are used to amplify a given target, and where a battery of primers are utilized, for example in cases of sequence variability, as exemplified herein for the forward and reverse primers of the IMP amplification. Examples of batteries of primers include a single forward primer in conjunction with multiple reverse primers, a single reverse primer in conjunction with multiple forward primers, and multiple forward primers in conjunction with multiple reverse primers, for example as exemplified herein with the IMP primers.

“Channel” as used herein refers to a range of emission wavelengths of a fluorophore. Examples of channels are those used herein, namely FAM, whose emission peak is at 520 nanometers (nm), referred to herein as the green channel, HEX, whose emission peak is at 556 nm, referred to herein as the yellow channel, Cal Fluor® Red 610, whose emission peak is at 610 nm, referred to herein as the orange channel, Quasar® 670, whose emission peak is at 670 nm, referred to herein as the red channel, and Quasar® 705, whose emission peak is at 705 nm, referred to herein as the crimson channel. Those skilled in the art will appreciate, based on the present disclosure, that the present invention is not in any way constrained by the choice of particular channels utilized herein. Different channels may be substituted, or, in other embodiments, additional channels may be added.

Hot-Start Primers

In some embodiments, the described compositions and methods utilize hot-start primers. In certain embodiments, the hot-start primers include an inactivating chemical modification that is reversed by the action of an activating enzyme present in the amplification mixture. Some examples of inactivating modifications are 3′ blocking groups and 3′ dideoxy nucleotides, in combination with an internal feature several bases away, which is cleaved by the action of a thermophilic activating enzyme, where the hot-start primers become a substrate for the thermophilic activating enzyme only when the primers are hybridized, in some embodiments stably hybridized, to a complementary sequence at elevated temperatures. The blocking group is thus removed by the action of the activating enzyme. A non-limiting example of this is “ribo-primers”, which are described in more detail hereinbelow.

In general, reference to primer sets as being “hot-start” does not preclude embodiments where a mixture of otherwise identical hot-start and non-hot-start primers are utilized. In some embodiments, spiking a hot-start primer or primer set with a relatively small amount of non-hot-start primers may help overcome PCR inhibition.

Another type of hot-start primers is described in U.S. Pat. No. 6,482,590 to Edwin Ullman et al, assigned to Aventis Behring GmbH, entitled “Method for polynucleotide amplification”, which describes modified oligonucleotides having a 3′-end that is inefficiently extendable along any polynucleotide. The modified nucleotides are reportedly relatively resistant to the 3′-exonuclease activity of Pfu polymerase activity at ambient temperatures, but undergo slow degradation to remove the modified nucleotides as the temperature is increased, resulting in gradual introduction of functional primers into the PCR reaction, thereby improving overall specificity.

US Pat. App. No. 2007/0128621, assigned to Applera Corporation, describes PCR reaction mixtures for multiplex amplification of mRNA and micro RNA targets, containing a hot-start primer having a stem-loop structure and directed against the mRNA target and a regular primer directed against the micro RNA target.

US Pat. App. No. 2007/0281308 to Gerald Zon et al, entitled “Chemically Modified Oligonucleotide Primers for Nucleic Acid Amplification”, discloses primers containing a heat-removable modification group, preferably at the 3′ terminus, which dissociates during the initial denaturation step of the amplification. The inactive primers may be present in a mixed population with functional primers.

International patent application WO 2009/004630 to Ofer Peleg of Genaphora Ltd, entitled “Chimeric Primers for Improved Nucleic Acid Amplification Reactions”, and an article by Peleg et al (The use of chimeric DNA/RNA primers in quantitative PCR for the detection of Ehrlichia canis and Babesia canis. Appl Environ Microbiol. 2009; 75(19):6393-8) describe another type of primer used for reducing non-specific amplification reactions. These primers, which incorporate a few ribonucleotides in non-adjacent positions in proximity to the initiation zone, reportedly decrease the formation of non-specific amplification products.

Another type of hot-start primers is described in articles by M Ailenberg et al. (Controlled hot start and improved specificity in carrying out PCR utilizing touch-up and loop incorporated primers (TULIPS). Biotechniques. 2000; 29(5):1018-20, 1022-4) and O K Kaboev et al (PCR hot start using primers with the structure of molecular beacons (hairpin-like structure). Nucleic Acids Res. 2000; 28(21):E94), which describe loop primers that contain additional non-template 5′ sequence that self-anneals to the 3′ region and inhibits initiation of polymerization. Upon heating of the reaction mixture, the loop regions of the primers reportedly melt and are activated.

Hot-start primers containing covalent chemical modifications are also described in the literature. US Pat. App. No. 2003/0119150 to Waltraud Ankenbauer et al, assigned to Roche Diagnostics, entitled “Composition and method for hot start nucleic acid amplification”, describes use of primers containing chemical modifications at the 3′ end of at least one primer. The reaction mixture also includes a thermostable exonuclease that is inactive at ambient temperatures, thus leaving the modified primer unaffected. When the temperature is increased, the exonuclease becomes active and removes the 3′ modification of the primer, activating the primer for amplification.

US Pat. App. No. 2003/0162199 to Alex Bonner, assigned to BioLink Partners, Inc, entitled “Reversible chemical modification of nucleic acids and improved method for nucleic acid hybridization” describes modification of target nucleic acid(s), primer(s) or nucleoside triphosphates with a removable protecting group that is releasable from the nucleic acids using heat. The chemical modification can be selected from glyoxal, derivatives thereof, 3,4,5,6-tetrahydrophthalic anhydride, 3-ethoxy-2-ketobutyraldehyde(kethoxal), ninhydrin, hydroxyacetone, diethyl oxalate, diethyl mesoxalate, 1,2-naphthoquinone-4-sulfonic acid, pyruvaldehyde, amides, γ-carboxyacylamides, amidines, and carbamates.

A V Lebedev et al. (Hot Start PCR with heat-activatable primers: a novel approach for improved PCR performance. Nucleic Acids Res. 2008. 36(20):e131) describes another type of hot start primers that contain 1-2 thermolabile, 4-oxo-1-pentyl (OXP) phosphotriester (PTE) modification groups at 3′-terminal and 3′-penultimate inter-nucleotide linkages. These modifications reportedly impair DNA polymerase primer extension under pre-reaction conditions. Incubation of the OXP-modified primers at elevated temperatures yields the corresponding unmodified phosphodiester (PDE) primer, which is a suitable DNA polymerase substrate.

U.S. Pat. No. 6,794,142, to Walter J. Laird et al, assigned to Roche Molecular Systems, Inc, entitled “Amplification using modified primers”, describes hot-start primers containing a modified nucleotide within the three 3′ terminal nucleotide positions; wherein the modified nucleotide is a 2′-O-methyl nucleotide, 2′-fluoro-nucleotide, 2′-amino nucleotide, or arabinose nucleotide. These modified primers reportedly reduce non-specific amplification by increasing the time required for the initial primer extension to occur, probably by rendering the primer-target duplex a less preferred template for extension. This reduces the likelihood that an unstable, transient hybridization duplex, such as between primers under pre-reaction conditions, will exist for a sufficient time to permit primer extension.

A different type of hot-start primers is described by D D Young et al (Light-triggered polymerase chain reaction. Chem Commun (Camb). 2008; (4):462-4). These primers are modified with a sterically demanding caging group that is removable by UV irradiation. The unmodified primers reportedly fail to catalyze a PCR reaction until exposed to UV irradiation, after which the reaction proceeds normally. Such primers are suitable for a hot-start protocol wherein the reaction mixture is heated to the annealing temperature, then exposed to UV irradiation.

“Ribo-primers”: US Pat. App. Nos. and 2009/0325169 and 2010/0167353, both assigned to Integrated DNA Technologies Inc. (IDT) and entitled “RNase H-Based Assays Utilizing Modified RNA Monomers”, describe another type of hot-start PCR primers, “ribo-primers”. The modified primers have an internal RNA base that generates an RNase H2 cleavage site when bound to DNA. In addition, the primers contain a 3′ blocking group, which precludes the ability of the primers to support PCR until the blocking group is removed. These primers are suitable for PCR reaction mixes containing a thermostable RNase H2, which cleaves internal RNA bases from a mostly DNA-DNA hybrid at the elevated temperatures employed in the reaction, or another endonuclease with similar activity. Cleavage by a thermophilic RNase H2 (for example for example Pyrococcus abyssi Ribonuclease H2 endonuclease [RNAse H2]) requires stable duplex formation of primer and target at elevated temperature, and thus is quite mismatch sensitive. For example, P. abyssi RNAse H2 exhibits minimal activity below 50° C., with peak activity around 70° C. (Dobosy et al). Thus, duplex formation at temperatures of 50° C. or higher are required for appreciable amplification in this system. This increases the specificity of priming, which reduces the impact of primer-dimer formation, lowering the background signal and improving the overall reaction specificity.

A number of other references describe use of non-functional or antagonistic primers in nucleic acid amplification reactions. US Pat. App. No. 2003/0104430 and International Application WO00/61817 to Michael Nerenberg et al, entitled “Amplification and separation of nucleic acid sequences using strand displacement amplification and bioelectronic microchip technology”, for example, describe use of non-cleavable primers in a primer mix for strand displacement amplification (SDA), in combination with bioelectronic microchip technology. SDA is an isothermal and asynchronous nucleic acid amplification process. The non-cleavable primers are intended to retain signal that was been nicked prior to denaturation of the double-stranded template, thus improving signal intensity in anchored SDA, or to bias amplification towards a desired direction. The non-cleavable primers may be provided in combination with normal SDA primers.

U.S. Pat. No. 5,712,386 to Chang-Ning Wang et al, assigned to Biotronics Corporation, discloses blocking nucleotides that hybridize to primers. The blocking nucleotides and primers may be present in a molar ratio of blocking nucleotide/primer of between 0.3-5.0.

Shared-Stem Probes

Unless explicitly defined otherwise:
The term “partial shared-stem probe” refers herein to a probe in which at least 25% of the nucleotide residues of one strand of the stem structure are also complementary to its target nucleotide sequence. The mismatches to the target sequence may be on the internal end of the stem, in the middle of the stem sequence, on the end of the probe, or any combination thereof. In this definition and all the following definitions of shared-stem probes, the “target sequence” refers to the sequence desired to be detected by the target. If the target sequence has known variants, this term refers to the most common variant thereof.
The term “majority shared-stem probe” refers herein to a probe in which the majority of the nucleotide residues of one strand of the stem structure are also complementary to its target nucleotide sequence. The mismatches to the target sequence may be on the internal end of the stem, in the middle of the stem sequence, on the end of the probe, or any combination thereof.
The term “fully shared-stem probe” refers herein to a probe in which all the nucleotide residues of one strand of the stem structure are also complementary to its target nucleotide sequence.
The term “double, partial shared-stem probe” refers herein to a probe in which at least 25% of the nucleotide residues of each strand of the stem structure are also complementary to its target nucleotide sequence. The mismatches to the target sequence may be on the internal end of the stem, in the middle of the stem sequence, on the end of the probe, or any combination thereof
The term “double, majority-stem probe” refers herein to a probe in which the majority of the nucleotide residues of each strand of the stem structure are also complementary to its target nucleotide sequence. The mismatches to the target sequence may be on the internal end of the stem, in the middle of the stem sequence, on the end of the probe, or any combination thereof
The term “double, fully shared-stem probe” refers herein to a probe in which all the nucleotide residues of both strands of the stem structure are also complementary to its target nucleotide sequence.
The term “double shared-stem probe” refers herein to any or all of the preceding three definitions, with each definition being a separate embodiment.
The term “shared-stem probe” refers herein to any or all of the preceding seven definitions, with each definition being a separate embodiment.

Asymmetric Primer Sets

As used herein, an “asymmetric” primer set is a primer set in which either the forward primer(s) or the reverse primer(s) are intentionally present in excess quantities, and the primer(s) of the other direction is present in limiting quantities, relative to the amounts that would be used for symmetric PCR. As provided herein, this may be done to facilitate preferential linear-after-exponential amplification of one strand of the PCR product (the “excess strand”). In some embodiments, the concentration of the excess primer is at least 5-fold as much as the limiting primer. As a non-limiting example, the excess primer may be present at a concentration of 0.7-1.5 micromolar, and the limiting primer present at 0.07-0.15 micromolar in other embodiments 0.07-0.2 micromolar. In light of the present disclosure and Rice et al, Gentile et al, and US Pat. Appl. Publ. No. 2004/0053254 to Wangh et al and the references cited therein, those skilled in the art will be able to readily determine the appropriate concentrations of the excess and limiting primers for a given set of reaction conditions.

Internal T_{M}, Hybrid T_{M}, and Delta T_{M}

The term “ΔT_{M}” as used herein is difference between the internal melting temperature (T_{M}) of the stem of the probe (the “internal T_{M}”) and the T_{M} of a hybrid of the probe with the target sequence that is desired to be detected (the “hybrid T_{M}”), where a positive number indicates a higher T_{M} of the probe stem. Unless indicated otherwise, both parameters are measured under PCR reaction conditions, namely 60 mM KCl, 7 mM MgCl₂, 3.2 mM each of the dNTPS, at a probe concentration of 0.125 micromolar, pH 8.3.

Fluorescence Signatures

Reference herein to target-probe fluorescence “signature(s)” (also referred to as “hybrid fluorescence signature(s)”) indicates the target-probe peak melting temperature (T_{M}), the shape of the fluorescence curve upon controlled melting of the target-probe hybrid or controlled annealing of the probe to the target, or a combination of the T_{M} and the shape of the curve. Those of skill in the art will appreciate, in light of the information and exemplification provided herein, that target-probe fluorescence signatures can be discriminated from one another by visual inspection of the fluorescence curves and/or mathematical processing of the data. In certain embodiments, it is preferred that the peak T_{M} of discriminable peaks differ by at least 5° C., or in other embodiments at least 3° C., or in various embodiments at least 4° C., at least 2° C., at least 5° C., at least 6° C., at least 7° C., or at least 8° C.

Embodiments of Reaction Mixtures

Provided herein is a reaction mixture, comprising: (a) a nucleotide-containing test sample (e.g. a DNA extract of a blood sample from a human); (b) 6 or more primer sets, wherein at least the majority of (most or all of) the primer sets, or in other embodiments every primer set, is asymmetric; and (c) 6 or more probes, which fluoresce in 4 or more different channels, wherein the following are true:
each of the probes binds to a polynucleotide selected from (i) a PCR product of a target amplified by one or more of the primer sets, typically the excess strand of a PCR product in the case of asymmetric amplification; and (ii) a control polynucleotide, whereupon fluorescence of the probe is activated;
in at least one of the channels, a plurality of different target-probe fluorescence signatures are discriminable; and
the forward and reverse primers of each of at least the majority of (most or all of) the primer sets are hot-start primers. In other embodiments, all the primers in the reaction mixture are hot-start primers.

Typically, the aforementioned reaction mixture is indicated for amplification and detection in a single reaction tube. In other embodiments, the mixture is provided in a single reaction tube.

Also provided herein is a reaction mixture, either present in a single PCR reaction tube or split into two PCR reaction tubes, or in other embodiments more than two PCR reaction tubes, comprising:
A. a test sample suspected to contain one or more of a set of target polynucleotide sequences;
B. a group of primer sets that amplify the set of targets, where the targets comprise each of the following:
   at least one Staphylococcus aureus (SA) marker polynucleotide;
   a polynucleotide selected from a non-SA Staphylococcus marker polynucleotide and a general Staphylococcus marker polynucleotide;
   an Enterococcus marker polynucleotide (in some embodiments, a marker for E. faecium and E. faecalis);
   an alpha-hemolytic Streptococcus marker polynucleotide (non-limiting embodiments of which are S. pneumoniae marker polynucleotides),
   at least one nucleotide sequence associated with vancomycin resistance; and
   at least one nucleotide sequence associated with methicillin resistance; and
C. probes that collectively fluoresce in 4 or more different channels, meaning that while each probe will typically fluoresce in a particular channel, the various probes present have 4 or more different peak fluorescence wavelengths among them,
   wherein each of the probes binds to a polynucleotide selected from (i) a PCR product of at least one of the set of targets, which is in some embodiments the excess strand of a PCR product in the case of asymmetric amplification, and (ii) a control polynucleotide, whereupon fluorescence of the probe is activated. In some embodiments, at least the majority of the primer sets in the reaction mixture are hot-start primers. In other embodiments, all the primers in the reaction mixture are hot-start primers. In still other embodiments, the reaction mixture further comprises an internal control polynucleotide and a probe for detecting same, and in yet other embodiments also primers for amplifying the internal control polynucleotide. Those skilled in the art will appreciate that the reaction mixture will typically further comprise a nucleotide-containing test sample (e.g. a DNA extract of a blood sample from a human).

In general, when reference is made to a reaction mixture that is split into 2 or more tubes, the intention, in some embodiments, is that test sample is split into several aliquots, with each aliquots being combined with certain sets of primers and their corresponding probes, as well as the other (non-specific) components of the reaction mixture. In still other embodiments, it is feasible to use more than 2 reaction tubes, for example if more blood is available, or the efficiency of sample preparation is increased. The described compositions and methods are not intended to be limited to reaction mixtures in 2 or fewer tubes.

Provided, in addition, are reaction mixtures, either present in a single reaction tube or split into several reaction tubes, comprising: (a) a test sample suspected to contain one or more of a set of target polynucleotide sequences; (b) a group of primer sets that amplify the set of targets, where the targets comprise: at least one marker polynucleotide of a gram-positive (GP) bacteria; and at least one antibiotic-resistance polynucleotide; and (c) a helicase enzyme. In certain embodiments, at least the majority, in other embodiments all, of the aforementioned primer sets are ribo-primers, and the reaction mixture further comprises an RNAse H2 enzyme. In other embodiments, the GP marker polynucleotides comprise at least one of: an SA marker; an Enterococcus marker; and an alpha-hemolytic Streptococcus marker (non-limiting embodiments of which are S. pneumoniae marker). Alternatively or in addition, the antibiotic-resistance polynucleotides comprise at least one of: a vancomycin-resistance polynucleotide and a methicillin-resistance polynucleotide. In more specific embodiments, the GP marker polynucleotides comprise an SA marker, a marker for E. faecium and E. faecalis, and an S. pneumoniae marker; and the antibiotic-resistance polynucleotides comprise a vancomycin-resistance polynucleotide and a methicillin-resistance polynucleotide. In certain embodiments, the various probes are discriminated from one another using a logic table that combines the identification of the probe color that showed positive in the qPCR phase with the Tm value that was detected in a subsequent controlled melt.

The aforementioned reaction mixtures are non-limiting examples of mixtures that focus on gram-positive bacterial markers, but are not necessarily limited to gram-positive bacterial markers. Such mixtures may be referred to as “gram-positive reaction mixtures”.

In some embodiments, the Enterococcus marker is the 16S gene (representative GenBank sequence accession number FJ378704 [accessed on Nov. 14, 2013]). In further embodiments, the 16S probe is one or both of 16S-ent-PB1 and 16S-ent-PB2 (SEQ ID NOs 22 and 121).

Alternatively or in addition, the S. pneumoniae marker may be the Spn9802 region of the genome (representative sequence accession number FQ312041 [accessed on Nov. 14, 2013]). In some embodiments, the Spn9802 probe is one or both of Spn9802-ent-PB1 and Spn9802-PB2 (SEQ ID NOs 23 and 24).

In certain embodiments, the aforementioned probes fluoresce in 4-7 different channels, in other embodiments in 4-6 different channels, in other embodiments in 4-5 different channels, in other embodiments in 5-6 different channels, in other embodiments in 5-7 different channels, in other embodiments in 4 different channels, in other embodiments in 5 different channels, in other embodiments in 6 different channels, and in other embodiments in 7 different channels.

In still other embodiments, the targets of the reaction mixture further comprise a general gram-positive bacteria marker, in other embodiments a general bacteria marker, or in other embodiments both a general gram-positive bacteria marker and a general bacteria marker. Non-limiting examples of such markers are provided herein, in the Experimental Details section.

In yet other embodiments, the targets further comprise a marker polynucleotide for Group A, C, and/or G beta-hemolytic Streptococcus. This marker may detect, in various embodiments, S. pyogenes, S. dysgalactiae, or S. canis, or in other embodiments any combination of two of these species, or in other embodiments all 3 of these species.

In still other embodiments, the targets further comprise an additional SA marker polynucleotide. In more specific embodiments, the SA marker polynucleotide and the additional SA marker polynucleotide are nuc and SPA (representative sequence accession numbers DQ399678 and EF455822, respectively [accessed on Nov. 14, 2013]). Those skilled in the art will appreciate in light of the present disclosure that, when certain members of a target pathogen do not contain a particular marker sequence, an additional marker sequence can be used for more complete detection of the pathogen. In further embodiments, the nuc and SPA are detected in the same channel. In more specific embodiments, the nuc and SPA probes may have similar hybrid T_{M}'s (e.g. within 2° C. of each other) with their desired target sequences. In some embodiments, the nuc probes are one or both of Nuc-PB and Nuc-PB2 (SEQ ID NOs. 75 and 124). In some embodiments, the SPA probes are one or both of SPA-PB and SPA-PB2 (SEQ ID NOs. 76 and 124).

Alternatively or in addition, the general Staphylococcus marker may be tuf, as exemplified herein (representative sequence accession number AF298798 [accessed on Nov. 14, 2013]). In some embodiments, the tuf probes are one or more of tuf-PB, tuf-PB2, tuf-PB3, and tuf-PB4 (SEQ ID NOs. 43-45 and 126).

Alternatively or in addition, the beta-hemolytic Streptococcus marker is Emm (representative sequence accession number DQ010932 [accessed on Nov. 14, 2013]). In some embodiments, the Emm probe is Emm-PB (SEQ ID NO. 79).

In some embodiments, the general GP bacteria marker and/or the general GN bacteria marker is the 16S gene (representative sequence accession numbers D83371 and AF233451, respectively [accessed on Nov. 14, 2013]). In further embodiments, the GP 16S probe is 16S-GP-PB (SEQ ID NO: 42). In still other embodiments, the GN 16S probe is 16S-GN-PB (SEQ ID NO: 11).

Alternatively or in addition, the Acinetobacter marker polynucleotide is rpoB (representative sequence accession number DQ207471 [accessed on Nov. 14, 2013]). In some embodiments, the rpoB probe is rpoB-PB (SEQ ID NO: 41).

Alternatively or in addition, the target nucleotide sequence(s) associated with vancomycin resistance is vanA, or in another embodiment vanB, or in another embodiment both vanA and vanB (representative sequence accession numbers GQ489013 and AY655711, respectively [accessed on Nov. 14, 2013]). In further embodiments, the vanA and vanB are detected in the same channel. In more specific embodiments, the vanA and vanB probes may have similar hybrid T_{M}'s (e.g. within 2° C. of each other) with their desired target sequences. Having close hybrid T_{M}'s maximizes, in some embodiments, their use in conjunction with one or more probes in the same channel, since there is a greater difference between the different hybrid T_{M}'s (or groups thereof) that are desired to be distinguishable. In some embodiments, the vanA probes are one or both of vanA-PB and vanA-PB2 (SEQ ID NOs. 77 and 129). In some embodiments, the vanB probes are one or both of vanB-PB and vanB-PB2 (SEQ ID NOs. 78 and 124).

In other embodiments, more than one probe that detects a nucleotide sequence associated with vancomycin resistance is present, and each of the probes fluoresces in the same channel as one another. In certain embodiments, the set of targets includes more than one nucleotide sequence associated with vancomycin resistance, and these sequences are detected in the same channel. In some embodiments, for example if this channel is limited to vancomycin resistance genes, this arrangement enables a readout of vancomycin resistance, or lack thereof, as soon as the amplification step has been completed, or shortly thereafter. Thus, the physician obtains valuable information that will guide antibiotic selection, without the need to wait until the controlled melt (or controlled annealing) is carried out.

Alternatively or in addition, the target nucleotide sequence(s) associated with methicillin resistance is at least one of mecA, or in another embodiment mecC, or in another embodiment both mecA and mecC (representative sequence accession numbers KF058908 and KC110686, respectively [accessed on Nov. 14, 2013]). In further embodiments, the mecA and mecC are detected in the same channel. In further embodiments, the mecA and mecC are detected in the same channel. In more specific embodiments, the mecA and mecC probes may have similar hybrid T_{M}'s (e.g. within 2° C. of each other) with their desired target sequences.

In still other embodiments, the target nucleotide sequences comprise at least 2 of mecA, mecC, vanA, and vanB. In still other embodiments, the markers comprise three or more of the aforementioned list. In yet other embodiments, the markers comprise all four of the aforementioned list. In other embodiments, the markers comprise both mecA and mecC in combination with at least one of vanA and vanB. In other embodiments, the markers comprise both vanA and vanB in combination with at least one of mecA and mecC. In some embodiments, the mecA probes are one or both of mecA-PB and mecA-PB2 (SEQ ID NOs. 73 and 122). In some embodiments, the mecC probes are one or both of mecC-PB and mecC-PB2 (SEQ ID NOs. 74 and 123).

In other embodiments, more than one probe that detects a nucleotide sequence associated with methicillin resistance is present, and each of the probes fluoresces in the same channel as one another. In certain embodiments, the set of targets includes more than one nucleotide sequence associated with methicillin resistance, and these sequences are detected in the same channel. In some embodiments, for example if this channel is limited to methicillin resistance genes, this arrangement enables a readout of methicillin resistance, or lack thereof, as soon as the amplification step has been completed, or shortly thereafter. Thus, the physician obtains valuable information that will guide antibiotic selection, without the need to wait until the controlled melt (or controlled annealing) is carried out. Other sets of targets that lead to the same recommendation, for example nuc and SPA, may be used together in the same manner.

In still other embodiments, one or more of the specific probes described herein from the GP panel are used, each combination of which is considered a separate embodiment.

In other embodiments, the targets of the aforementioned reaction mixtures may further comprise a Pseudomonas marker polynucleotide.

Alternatively or in addition, the targets may further comprise one or more fungus marker polynucleotides. In more specific embodiment, the fungus marker polynucleotides comprise one or more polynucleotides selected from: an Aspergillus marker, a general fungal marker, a general Candida and Aspergillus marker, and a C. albicans marker. In more specific embodiments, the Aspergillus marker may be an A. fumigatus marker. In other embodiments, the marker polynucleotides comprise two or more of the aforementioned list. In still other embodiments, the markers comprise three or more of the aforementioned list. In yet other embodiments, the markers comprise all four of the aforementioned list.

In more specific embodiments, the one or more fungus marker polynucleotides comprise at least one of: L1A1, gene encoding an 18S ribosomal RNA (rRNA), and a gene encoding a 28S rRNA (representative sequence accession numbers FJ159482, KC936147, and JQ301899, respectively [accessed on Nov. 14, 2013]). In other embodiments, the marker polynucleotides comprise two or more of the aforementioned list. In still other embodiments, the markers comprise all three of the aforementioned list. In some embodiments, the fungus probes are one or more of 28S-Aspergillus-PB, 18S fungus-PB, L1A1-PB, and 28S-CA-PB (SEQ ID NOs. 69-72).

In still other embodiments, the various fungus marker polynucleotides are all detected in the same channel, enabling a readout of fungal infection as soon as the amplification is completed, or shortly thereafter.

In still other embodiments, a single PCR reaction tube is provided, which comprises primer sets and probes for each of the following set of targets:
at least one Staphylococcus aureus (SA) marker polynucleotide;
a polynucleotide selected from a non-SA Staphylococcus marker polynucleotide and a general Staphylococcus marker polynucleotide;
an Enterococcus marker polynucleotide, for example a marker of E. faecium and E. faecalis;
an alpha-hemolytic Streptococcus marker polynucleotide (non-limiting embodiments of which are S. pneumoniae marker polynucleotides),
at least one nucleotide sequence associated with vancomycin resistance; and
at least one nucleotide sequence associated with methicillin resistance.

In more specific embodiments, the single tube further comprises primers and probes for S. pyogenes, S. dysgalactiae, and/or S. canis. Alternatively or in addition, the single tube further comprises primers and probes for an additional SA marker polynucleotide. Alternatively or in addition, the single tube further comprises primers and probes for one or more fungus marker polynucleotides, for example L1A1, an 18S rRNA, and 28S rRNA. Alternatively or in addition, the methicillin resistance marker is mecA or mecC, or in another embodiment both mecA and mecC Alternatively or in addition, the vancomycin resistance marker is vanA or vanB, or in another embodiment both vanA and vanB.

In other, more specific embodiments, the single tube comprises primers and probes for: an SA marker polynucleotide; a non-SA Staphylococcus marker polynucleotide or general Staphylococcus marker polynucleotide; a marker of E. faecium and E. faecalis; an S. pneumoniae marker polynucleotide; vanA and/or vanB; and mecA and/or mecC. In still other embodiments, the single tube comprises primers and probes for an SA marker polynucleotide; a non-SA Staphylococcus marker polynucleotide or general Staphylococcus marker polynucleotide; a marker of E. faecium and E. faecalis; an S. pneumoniae marker polynucleotide; vanA; vanB; mecA; and mecC. In yet other embodiments, the single tube comprises primers and probes for an SA marker polynucleotide; a non-SA Staphylococcus marker polynucleotide or general Staphylococcus marker polynucleotide; a marker of E. faecium and E. faecalis; an S. pneumoniae marker polynucleotide; a Pseudomonas marker polynucleotide, vanA and/or vanB; and mecA and/or mecC. In other embodiments, the single tube comprises primers and probes for an SA marker polynucleotide; a non-SA Staphylococcus marker polynucleotide or general Staphylococcus marker polynucleotide; a marker of E. faecium and E. faecalis; an S. pneumoniae marker polynucleotide; a Pseudomonas marker polynucleotide; vanA; vanB; mecA; and mecC. Alternatively or in addition, the general Staphylococcus marker may be tuf, as exemplified herein.

In still other embodiments, a reaction mixture is provided, either present in a single PCR reaction tube or split into two PCR reaction tubes, or in other embodiments more than two PCR reaction tubes, comprising primers and probes for some or all of the aforementioned GP bacterial targets, or in another embodiment the aforementioned GP bacterial and fungal targets, and in addition at least two of: (a) a general gram-negative bacteria marker polynucleotide; (b) a metallo-β-lactamase nucleotide sequence; (c) a serine-β-lactamase nucleotide sequence; and (d) an extended-spectrum-β-lactamase nucleotide sequence. In other embodiments, the marker polynucleotides comprise two or more of the aforementioned list. In still other embodiments, the markers comprise three or more of the aforementioned list. In yet other embodiments, the markers comprise all four of the aforementioned list. In still other embodiments, the reaction mixture further comprises an internal control polynucleotide and a probe for detecting same, and in yet other embodiments also primers for amplifying the internal control polynucleotide. This reaction mixture may be referred to as a “Gram-Positive and Gram Negative [or GP and GN] detection kit” or, if fungal targets are present, a “GP, GN, and fungal detection kit”.

Also provided herein is a reaction mixture, either present in a single PCR reaction tube or split into two PCR reaction tubes, or in other embodiments more than two PCR reaction tubes, comprising:
A. a test sample;
B. a group of primer sets that amplify a set of targets, where the targets comprise:
   a general gram-negative bacteria marker polynucleotide;
   a metallo-β-lactamase nucleotide sequence;
   a serine-β-lactamase nucleotide sequence; and
   a nucleotide sequence of a β-lactamase selected from a subgroup 2be β-lactamase and a subgroup 2br β-lactamase, non-limiting examples of which are 2be and 2br SHV β-lactamases; and
C. probes that collectively fluoresce in 4-7 different channels,
   wherein each of the probes binds to a polynucleotide selected from (i) a PCR product of at least one of the set of targets, in some embodiments the excess strand of a PCR product, in the case of asymmetric amplification; and (ii) a control polynucleotide, whereupon fluorescence of the probe is activated. In some embodiments, at least the majority of the primer sets in the reaction mixture are hot-start primers. In other embodiments, all the primers in the reaction mixture are hot-start primers. In still other embodiments, the reaction mixture further comprises an internal control polynucleotide and a probe for detecting same, and in yet other embodiments also primers for amplifying the internal control polynucleotide. Those skilled in the art will appreciate that the reaction mixture will typically further comprise a nucleotide-containing test sample (e.g. a DNA extract of a blood sample from a human).

Also provided herein is a kit, comprising the described GP reaction mixture and the described GN reaction mixture. In other embodiments, the kit comprises the described GP+fungal reaction mixture and the described GN reaction mixture.

Provided, in addition, are reaction mixtures, either present in a single reaction tube or split into several reaction tubes, comprising: (a) a test sample suspected to contain one or more of a set of target polynucleotide sequences; (b) a group of primer sets that amplify the set of targets, where the targets comprise: at least one marker polynucleotide of a gram-negative bacteria; and at least one antibiotic-resistance polynucleotide; and (c) a helicase enzyme. In certain embodiments, at least the majority, in other embodiments all, of the aforementioned primer sets are ribo-primers, and the reaction mixture further comprises an RNAse H2 enzyme. In other embodiments, the GN marker polynucleotide is a general GN marker polynucleotide. Alternatively or in addition, the antibiotic-resistance polynucleotides comprise at least one of: a metallo-β-lactamase sequence, a serine-β-lactamase nucleotide sequence, a subgroup 2be β-lactamase, and a subgroup 2br β-lactamase. In more specific embodiments, the GN marker polynucleotide is a general GN marker polynucleotide; and the antibiotic-resistance polynucleotides comprise a metallo-β-lactamase sequence, a serine-β-lactamase nucleotide sequence, a subgroup 2be β-lactamase, and a subgroup 2br β-lactamase. In certain embodiments, the various probes are discriminated from one another using a logic table that combines the identification of the probe color that showed positive in the qPCR phase with the Tm value that was detected in a subsequent controlled melt (or controlled annealing).

In still other embodiments is provided a kit, comprising a described helicase-containing GP reaction tube and a described helicase-containing GN reaction tube. In other embodiments, the kit comprises a described helicase-containing GP+fungal reaction tube and a described helicase-containing GN reaction tube.

The aforementioned reaction mixtures are non-limiting examples of mixtures that focus on gram-negative bacterial markers, but are not necessarily limited to gram-positive bacterial markers. Such mixtures may be referred to as “gram-negative reaction mixtures”.

In still other embodiments, one or more of the specific probes described herein from the GN panel are used, each combination of which is considered a separate embodiment.

In certain embodiments, the aforementioned probes fluoresce in 4-7 different channels, in other embodiments in 4-6 different channels, in other embodiments in 4-5 different channels, in other embodiments in 5-6 different channels, in other embodiments in 5-7 different channels, in other embodiments in 4 different channels, in other embodiments in 5 different channels, in other embodiments in 6 different channels, and in other embodiments in 7 different channels.

In other embodiments, the list of targets of the aforementioned GN reaction mixtures may further comprise a general gram-positive bacteria marker, or in other embodiments, a general bacteria marker. In still other embodiments the list of targets of the reaction mixtures further comprises a general gram-positive bacteria marker and a general bacteria marker.

In yet other embodiments, the list of targets further comprises an Acinetobacter marker polynucleotide.

The aforementioned metallo-β-lactamase is, in some embodiments, at least one of IMP-1, IMP-2, IMP-3, and IMP-4 (representative sequence accession numbers EU588392, AY055216, KC310496, and JQ407409, respectively) or is another IMP (representative sequence accession numbers HQ438058 and FJ655384) (the aforementioned entries were all accessed on Nov. 14, 2013). In other embodiments, the metallo-β-lactamase probe(s) detects all four of these IMP isoforms. In some embodiments, the IMP probes are one or both of IMP-PB 1 and IMP-PB2 (SEQ ID NOs. 91-92).

Alternatively or in addition, the Pseudomonas marker polynucleotide may be oprI (representative sequence accession number JF901402 [accessed on Nov. 14, 2013]). In some embodiments, the oprI probe is oprI-PB1 (SEQ ID NO. 93).

In other embodiments, the metallo-β-lactamase is vim (representative sequence accession number FM179468 [accessed on Nov. 14, 2013]). In some embodiments, the vim probes are one or both of VIM-PB1 and VIM-PB2 (SEQ ID NOs. 7-8).

In other embodiments, the metallo-β-lactamase is at least one of NDM-1, NDM-2, NDM-3, NDM-4, NDM-5, NDM-6, and NDM-7. In other embodiments, the metallo-β-lactamase probe(s) detects all seven of these NDM isoforms. In some embodiments, the NDM probes are one or more of NDM-PB1, NDM-PB2, and NDM-PB3 (SEQ ID NOs. 9, 10, and 100).

In still other embodiments, the set of metallo-β-lactamase primers and probes amplify and detect all of the following targets: IMP-1, IMP-2, IMP-3, and IMP-4, vim, NDM-1, NDM-2, NDM-3, NDM-4, NDM-5, NDM-6, and NDM-7.

Alternatively or in addition, the aforementioned serine-β-lactamase is at least one of KPC-2 (representative sequence accession number AY034847 [accessed on Nov. 14, 2013]), KPC-3, KPC-4, KPC-5, KPC-6, KPC-7, KPC-8, KPC-9, KPC-10, KPC-11. In other embodiments, the serine-β-lactamase probe(s) detects all 11 of these KPC isoforms. In some embodiments, the KPC probe is KPC-PB (SEQ ID NO. 38).

In other embodiments, the serine-β-lactamase is GES (In58 beta-lactamase IBC-2; representative sequence accession number AF329699 [accessed on Nov. 14, 2013]). In some embodiments, the GES probe is GES-PB (SEQ ID NO 39).

In other embodiments, the serine-β-lactamase is OXA-48 (K. pneumoniae strain 11978 insertion sequence IS1999; representative sequence accession number AY236073 [accessed on Nov. 14, 2013]). In some embodiments, the OXA-48 probe is OXA-48-PB (SEQ ID NO 40).

In still other embodiments, the set of serine-β-lactamase primers and probes amplify and detect all of the following targets: KPC-2, KPC-3, KPC-4, KPC-5, KPC-6, KPC-7, KPC-8, KPC-9, KPC-10, KPC-11, GES, and OXA-48.

Alternatively or in addition, the aforementioned extended-spectrum- or broad-spectrum-β-lactamase is at least one of a subgroup 2be or 2br variant of SHV lactamase, which are sometimes referred to in the scientific literature as extended-spectrum and broad-spectrum β-lactamases, respectively. SHV-2 and SHV-5, representative sequence accession numbers AF148851 and X55640, respectively [accessed on Nov. 14, 2013]) and SHV-12 are non-limiting examples of 2be lactamases. SHV-10 and SHV-72 are non-limiting examples of 2br lactamases. In other embodiments, the set of probes detects all of: SHV-2, SHV-3, SHV-10, SHV-72, and SHV-115. In other embodiments, the SHV probe is SHV-PB (SEQ ID NO 94).

In other embodiments, the extended-spectrum- or broad-spectrum-β-lactamase is CTXM-14, in other embodiments is CTXM-15, or in other embodiments is at least one of CTXM-14 and CTXM-15 (representative sequence accession numbers JQ003803 and JQ318855, respectively [accessed on Nov. 14, 2013]). In still other embodiments, both of these variants are amplified and detected by the primers and probes of the reaction mixture. In other embodiments, the CTXM-14 probe is CTXM-14-PB (SEQ ID NO 36). In other embodiments, the CTXM-15 probe is CTXM-15-PB (SEQ ID NO 37).

In still other embodiments, the set of primers and probes amplify and detect all of the following targets: SHV-2, SHV-3, SHV-10, SHV-72, and SHV-115, CTXM-14, and CTXM-15.

As used herein, the terms “subgroup 2be extended-spectrum-β-lactamase” and “subgroup 2br broad-spectrum-β-lactamase” as used as defined in Bush et al (Updated Functional Classification of β-Lactamases. Antimicrob Agents Chemother. 2010; 54(3): 969-976). This reference also contains antibiotic recommendations for various resistance genes.

In some embodiments, subgroup 2b β-lactamases are those that readily hydrolyze penicillins and early cephalosporins, such as cephaloridine and cephalothin, and are strongly inhibited by clavulanic acid and tazobactam. They include the TEM-1, TEM-2, and SHV-1 enzymes. Many TEM and SHV 2b enzymes have been described (G. Jacoby and K. Bush, http://www.lahey.org/Studies/).

In other embodiments, subgroup 2be enzymes retain the activity against penicillins and cephalosporins of subgroup 2b β-lactamases and in addition hydrolyze one or more oxyimino-β-lactams, such as cefotaxime, ceftazidime, and aztreonam, at a rate generally >10% that of benzylpenicillin. The first and largest subset of subgroup 2be was derived by amino acid substitutions in TEM-1, TEM-2, and SHV-1 that broadened their substrate spectrum at a cost of lower hydrolyzing activity for benzylpenicillin and cephaloridine. TEM and SHV ESBLs have been joined by the functionally similar but more rapidly proliferating CTXM enzymes that are related to chromosomally determined β-lactamases in species of Kluyvera. Most (but not all) CTXM enzymes hydrolyze cefotaxime more readily than ceftazidime. Many hydrolyze cefepime as well. Unlike TEM or SHV ESBLs, CTXM enzymes are inhibited by tazobactam at least an order of magnitude better than by clavulanic acid. Finally, there are less common ESBLs unrelated to TEM, SHV, or CTXM, including BEL-1, BES-1, SFO-1, TLA-1, TLA-2, and members of the PER and VEB enzyme families. Characteristically, subgroup 2be β-lactamases remain sensitive to inhibition by clavulanic acid.

In still other embodiments, subgroup 2br enzymes are broad-spectrum β-lactamases that have acquired resistance to clavulanic acid (IC₅₀≧1 μM) and related inhibitors while retaining a subgroup 2b spectrum of activity. Currently 36 of the 135 functionally characterized TEM enzymes have this property and include enzymes such as TEM-30 and TEM-31 (IRT-2 and IRT-1, respectively), as well as 5 of the corresponding functionally characterized 72 SHV enzymes (e.g., SHV-10) (G. Jacoby and K. Bush, http://www.lahey.org/Studies/).

In yet other embodiments, all of the following are true of the described GN reaction mixture:
A. the metallo-β-lactamase nucleotide sequence is at least one of IMP-1, IMP-2, IMP-3, IMP-4; vim, NDM-1, NDM-2, NDM-3, NDM-4, NDM-5, NDM-6, and NDM-7;
B. the serine-β-lactamase nucleotide sequence is at least one of KPC-2, KPC-3, KPC-4, KPC-5, KPC-6, KPC-7, KPC-8, KPC-9, KPC-10, KPC-11, GES, and OXA-48; and
C. the extended-spectrum-β-lactamase nucleotide sequence is at least one of SHV-2, SHV-3, SHV-10, SHV-72, SHV-115, CTXM-14, and CTXM-15.

In yet other embodiments, the described GN reaction mixture detects all of the following targets: IMP-1, IMP-2, IMP-3, IMP-4; vim, NDM-1, NDM-2, NDM-3, NDM-4, NDM-5, NDM-6, NDM-7; KPC-2, KPC-3, KPC-4, KPC-5, KPC-6, KPC-7, KPC-8, KPC-9, KPC-10, KPC-11, GES, OXA-48; SHV-2, SHV-3, SHV-10, SHV-72, SHV-115, CTXM-14, and CTXM-15.

In still other embodiments, a single PCR reaction tube is provided, which comprises primer sets and probes for the following set of targets:
a general gram-negative bacteria marker polynucleotide;
a metallo-β-lactamase nucleotide sequence;
a serine-β-lactamase nucleotide sequence; and
a nucleotide sequence of a β-lactamase selected from a subgroup 2be extended-spectrum-β-lactamase and a subgroup 2br broad-spectrum-β-lactamase, non-limiting examples of which are 2be and 2br SHV β-lactamases.

In other embodiments, the reaction mixture further comprises an internal control polynucleotide and a probe for detecting same. Alternatively in or addition, the set of targets of the single tube further comprises a general gram-positive bacteria marker. Alternatively in or addition, the set of targets may further comprise an Acinetobacter marker polynucleotide.

In still other embodiments, a single PCR reaction tube is provided, which comprises primer sets and probes for the following set of targets:
a general gram-negative bacteria marker polynucleotide;
a nucleotide sequence selected from: IMP-1, IMP-2, IMP-3, IMP-4; vim, NDM-1, NDM-2, NDM-3, NDM-4, NDM-5, NDM-6, and NDM-7;
a nucleotide sequence selected from: KPC-2, KPC-3, KPC-4, KPC-5, KPC-6, KPC-7, KPC-8, KPC-9, KPC-10, KPC-11, GES, and OXA-48; and
a nucleotide sequence selected from: SHV-2, SHV-3, SHV-10, SHV-72, SHV-115, CTXM-14, and CTXM-15.

In yet other embodiments, a single PCR reaction tube is provided, which comprises primer sets and probes that amplify and detect a general gram-negative bacteria marker polynucleotide and all of the following targets: IMP-1, IMP-2, IMP-3, IMP-4; vim, NDM-1, NDM-2, NDM-3, NDM-4, NDM-5, NDM-6, NDM-7, KPC-2, KPC-3, KPC-4, KPC-5, KPC-6, KPC-7, KPC-8, KPC-9, KPC-10, KPC-11, GES, OXA-48, SHV-2, SHV-3, SHV-10, SHV-72, SHV-115, CTXM-14, and CTXM-15.

In one embodiment, amplification reactions for such internal control loci are conducted in the same aliquot(s) of the reaction mixture as other amplification reactions for the described cycle threshold assay. In another embodiment, the internal control amplification reaction is conducted in a different aliquot of the reaction mixture than the other amplification reactions. For example, the internal control may be added to a tube containing no test sample DNA, only the other components of the assay.

Provided, in addition, is a reaction mixture, comprising: (a) test sample; and (b) at least one primer set, where, for at least one primer set in the reaction mixture, the following are true:
the primer set is asymmetric;
the forward and reverse primers of the primer set are hot-start primers that contain an inactivating chemical modification that is reversed by the action of an activating enzyme, where the primers become a substrate for the activating enzyme when the primers are hybridized to a complementary sequence, e.g. the target sequence, at elevated temperatures;
the melting temperature of the amplicon produced by extension of the primer set exceeds the initial, concentration-adjusted melting temperature (i.e. the T_{M} at the initial concentration of the primer in the reaction mixture) of a hybrid of the pre-cleavage excess primer and its target polynucleotide by more than 13° C.;
the initial, concentration-adjusted melting temperature of a hybrid of the pre-cleavage excess primer and its target polynucleotide is not above 73° C. (in other embodiments between 67-73° C., in other embodiments between 68-73° C., and in other embodiments between 69-73° C.); and
the initial, concentration-adjusted melting temperature of a hybrid of the post-cleavage excess primer and its target polynucleotide is at least 65° C., in other embodiments between 65-71° C., in other embodiments between 65-70° C., and in other embodiments between 65-69° C.

Also provided herein is a method of detecting the presence of a polynucleotide in a test sample, the method comprising the step of thermocycling a reaction mixture, while periodically measuring fluorescence at each of the channels, where the reaction mixture comprises: (a) a test sample; and (b) at least one primer set, where, for at least one primer set in the reaction mixture, the following are true:
the primer set is asymmetric;
the forward and reverse primers of the primer set are hot-start primers that contain an inactivating chemical modification that is reversed by the action of an activating enzyme, where the hot-start primers become a substrate for the activating enzyme when the hot-start primers are hybridized to a complementary sequence at elevated temperatures;
the melting temperature of the amplicon produced by the primer set exceeds the initial, concentration-adjusted melting temperature of a hybrid of the pre-cleavage excess primer and its target polynucleotide by more than 13° C.;
the initial, concentration-adjusted melting temperature of a hybrid of the pre-cleavage excess primer and its target polynucleotide is not more than 17° C. higher (in some embodiments 12-17° C. higher, in other embodiments 13-17° C. higher, in other embodiments 14-17° C. higher) than the annealing temperature of the thermocycling; and
the initial, concentration-adjusted melting temperature of a hybrid of the post-cleavage excess primer and its target polynucleotide is at least 9° C. higher (in some embodiments 9-15° C. higher, in other embodiments 9-14° C. higher, in other embodiments 9-13° C. higher) than the annealing temperature of the thermocycling.

The above temperatures of 73° C. and 65° C. are designed, in some embodiments, for a reaction with an annealing temperature of 56° C. If the annealing temperature is raised or lowered, these temperatures will be adjusted in the same manner.

In other embodiments, the above statements are true of at least the majority of the primer sets in the reaction mixture. In other embodiments, the above statements are true of at least the majority of the asymmetric primer sets in the reaction mixture. In other embodiments, the above statements are true of all the asymmetric primer sets in the reaction mixture.

In other embodiments, the above statements are true of at least the majority of the primer sets in the reaction mixture that amplify an amplicon whose GC content is at least 50%, in other embodiments at least 52.5%, in other embodiments at least 55%, in other embodiments at least 57.5%, in other embodiments at least 60%, in other embodiments at least 62.5%, in other embodiments at least 65%. In other embodiments, the above statements are true of all the primer sets in the reaction mixture that amplify an amplicon whose GC content is at least 55%, in other embodiments at least 57.5%, in other embodiments at least 60%, in other embodiments at least 62.5%, in other embodiments at least 65%.

In some embodiments, the aforementioned limitations on the pre-cleavage and post-cleavage T_{M}'s are also true of the limiting primer of the aforementioned primer set(s). The temperatures are measured at the initial concentration of the limiting primer, which of course is less than the excess primer.

Alternatively or in addition, the GC content of the amplicon of the aforementioned primer set(s) is at least 50%, in other embodiments at least 52.5%, in other embodiments at least 55% in other embodiments at least 57.5%, in other embodiments at least 60%, in other embodiments at least 62.5%, in other embodiments at least 65%.

In certain, more specific embodiments of the aforementioned reaction mixtures, the initial, concentration-adjusted melting temperature of the pre-cleavage limiting primer is at least as high as, in other embodiments at least 1° C. higher, in other embodiments at least 2° C. higher, in other embodiments at least 3° C. higher, in other embodiments at least 1° C. lower, in other embodiments at least 2° C. lower, or in other embodiments at least 3° C. lower than the initial, concentration-adjusted melting temperature of the pre-cleavage excess primer.

Alternatively or in addition, the aforementioned reaction mixtures may further comprise 6 or more probes, which fluoresce in 1-3 different channels, in other embodiments 4 or more channels, where each of the probes binds to a polynucleotide selected from (a) a PCR product of a target amplified by one or more of the primer sets; and (b) a control polynucleotide, whereupon fluorescence of the probe is activated. In further embodiments, a plurality of different target-probe fluorescence signatures may be discriminable in at least one of the channels.

In some embodiments, the aforementioned methods further comprise the steps of (a) subjecting the amplification product to a controlled heating or controlled cooling, while periodically measuring fluorescence at each of the channels; and (b) for each channel in which a signal is present and a plurality of different target-probe fluorescence signatures are discriminable, identifying the fluorescence signature that is present.

Also provided herein is a reaction mixture, comprising: (a) a test sample; and (b) at least one primer set, where, for at least one primer set in the reaction mixture, the following are true:
the primer set is asymmetric;
the forward and reverse primers of the primer set contain an inactivating chemical modification that is reversed by the action of an activating enzyme, where the primers become a substrate for the activating enzyme when the primers are hybridized to a complementary sequence at elevated temperatures;
the melting temperature of the amplicon produced by extension of the primer set exceeds the initial, concentration-adjusted melting temperature of a hybrid of the pre-cleavage excess primer and its target polynucleotide by more than 13° C.;
the GC content of the amplicon is at least 55%; and
the GC content of the region bound by the excess primer of the primer set is at least 1% lower, in other embodiments at least 2% lower, in other embodiments at least 3% lower, in other embodiments at least 4% lower, in other embodiments at least 5% lower, in other embodiments at least 6% lower, in other embodiments at least 7% lower, than the GC content of the amplicon.

In other embodiments, the above statements are true of at least the majority of the primer sets in the reaction mixture. In other embodiments, the above statements are true of at least the majority of the asymmetric primer sets in the reaction mixture. In other embodiments, the above statements are true of all the asymmetric primer sets in the reaction mixture.

In other embodiments, the above statements are true of at least the majority of the primer sets in the reaction mixture that amplify an amplicon whose GC content is at least 55%, in other embodiments at least 57.5%, in other embodiments at least 60%, in other embodiments at least 62.5%, in other embodiments at least 65%. In other embodiments, the above statements are true of all the primer sets in the reaction mixture that amplify an amplicon whose GC content is at least 55%, in other embodiments at least 57.5%, in other embodiments at least 60%, in other embodiments at least 62.5%, in other embodiments at least 65%.

In certain embodiments of the aforementioned reaction mixtures and methods, the following statements are also true:
the initial, concentration-adjusted melting temperature of a hybrid of the pre-cleavage excess primer and its target polynucleotide is not above 73° C.; and
the initial, concentration-adjusted melting temperature of a hybrid of the post-cleavage excess primer and its target polynucleotide is at least 65° C.

In some embodiments, the aforementioned limitations on the pre-cleavage and post-cleavage T_{M}'s are also true of the limiting primer of the aforementioned primer set(s). The temperatures are measured at the initial concentration of the limiting primer.

Alternatively or in addition, the aforementioned reaction mixtures may further comprise 6 or more probes, which fluoresce in 1-3 different channels, in other embodiments 4 or more channels, where each of the probes binds to a polynucleotide selected from (a) a PCR product of a target amplified by one or more of the primer sets; and (b) a control polynucleotide, whereupon fluorescence of the probe is activated. In further embodiments, a plurality of different target-probe fluorescence signatures may be discriminable in at least one of the channels.

Also provided herein is a method of detecting the presence of a polynucleotide in a test sample, the method comprising the step of thermocycling the aforementioned reaction mixtures, while periodically measuring fluorescence at each of the channels.

In some embodiments, the aforementioned methods further comprise the steps of (a) subjecting the amplification product to a controlled heating or controlled cooling, while periodically measuring fluorescence at each of the channels; and (b) for each channel in which a signal is present and a plurality of different target-probe fluorescence signatures are discriminable, identifying the fluorescence signature that is present.

It is mentioned above, in various embodiments, that the amplicon's T_{M} exceeds the initial, concentration-adjusted pre-cleavage T_{M} of the excess primer hybrid by more than 13° C. In some embodiments, the difference is more than 14° C., in other embodiments more than 15° C., in other embodiments more than 16° C., in other embodiments more than 17° C., in other embodiments more than 18° C., and in other embodiments more than 19° C.

Those skilled in the art will appreciate that T_{M}'s of primer-target hybrids can be determined inter alia using the Oligoanalyzer program, available from Integrated DNA technologies at (http://cu.idtdna.com/analyzer/applications/oligoanalyzer). For amplicon T_{M} prediction, the “Lasegene” software (DNASTAR—http://www.dnastar.com/) can be used. It can also be measured with SYBR Green (or another double strand DNA intercalating dye such as EvaGreen®).

In certain embodiments, every primer set utilized in the methods and reaction mixtures described herein is asymmetric. Typically, in these embodiments, every probe that binds to a PCR product will bind to the excess strand of the relevant PCR product. In other embodiments, at least the majority of primer sets in the reaction tube, or in each reaction tube if more than one is present, is asymmetric.

As mentioned, the described methods and compositions may utilize probes that collectively fluoresce in 4 or more different channels, meaning that while each probe will typically fluoresce in a particular channel, the various probes present have 4 or more different peak fluorescence wavelengths among them. In other embodiments, the probes fluoresce in 5 or more different channels. In other embodiments, the probes fluoresce in 4 different channels. In other embodiments, the probes fluoresce in 5 different channels. In other embodiments, the probes fluoresce in 6 different channels. In other embodiments, the probes fluoresce in 7 different channels. In other embodiments, the probes fluoresce in 4-10 different channels. In other embodiments, the probes fluoresce in 4-9 different channels. In other embodiments, the probes fluoresce in 4-8 different channels. In other embodiments, the probes fluoresce in 4-7 different channels. In other embodiments, the probes fluoresce in 4-6 different channels. In other embodiments, the probes fluoresce in 4 or 5 different channels. In other embodiments, the probes fluoresce in 5-10 different channels. In other embodiments, the probes fluoresce in 5-9 different channels. In other embodiments, the probes fluoresce in 5-8 different channels. In other embodiments, the probes fluoresce in 5-7 different channels. In other embodiments, the probes fluoresce in 5 or 6 different channels.

In other embodiments, at least 2 different target-probe fluorescence signatures are discriminable in each of at least 2 of the channels, meaning that the signals of 2 separate targets from the desired list of targets can be distinguished from one another in each of the channels. In other embodiments, at least 2 different fluorescence signatures are discriminable in each of at least 3 of the channels, or in other embodiments at least 4 of the channels, or in other embodiments at least 5 of the channels, in which the probes fluoresce. In other embodiments, at least 2 different fluorescence signatures are discriminable in each of 2 of the channels, or in other embodiments 3 of the channels, or in other embodiments 4 of the channels, or in other embodiments 5 of the channels, or in other embodiments all the channels. In other embodiments, at least 2 different fluorescence signatures are discriminable in each of the orange, red, and crimson fluorescence channels.

In other embodiments, at least 3 different target-probe fluorescence signatures are discriminable in each of at least 2 of the channels, or in other embodiments at least 3 of the channels, or in other embodiments at least 4 of the channels, or in other embodiments at least 5 of the channels, in which the probes fluoresce. In other embodiments, at least 3 different fluorescence signatures are discriminable in each of 2 of the channels, or in other embodiments 3 of the channels, or in other embodiments 4 of the channels, or in other embodiments 5 of the channels, or in other embodiments all the channels. In other embodiments, at least 3 different fluorescence signatures are discriminable in each of the orange, red, and crimson fluorescence channels. In still other embodiments, at least 3 different fluorescence signatures are discriminable in each of the orange, red, and crimson fluorescence channels, and at least 2 different fluorescence signatures are discriminable in each of the green and yellow channels. In yet other embodiments, at least 3 different fluorescence signatures are discriminable in each of the orange, red, and crimson fluorescence channels, and at least 2 different fluorescence signatures are discriminable in each of the remaining channels. In even more specific embodiments, 3 different fluorescence signatures are discriminable in each of the orange, red, and crimson fluorescence channels, and 1 or 2 of different fluorescence signatures are discriminable in each of the remaining channels.

In other embodiments of the aforementioned reaction mixtures, the probes in each channel in which at least 2 different target-probe fluorescence signatures are discriminable are considered as a group, and most or all of the probes in each of those channels is a shared-stem probe, or in other embodiments, is a fully shared-stem probe. In still other embodiments, every probe in each channel in which at least 2 different target-probe fluorescence signatures are discriminable is a shared-stem probe, or in other embodiments, is a fully shared-stem probe.

In yet other embodiments, the probes in each channel in which at least 3 different target-probe fluorescence signatures are discriminable are considered as a group, and most or all of the probes in each of those channels is a shared-stem probe, or in other embodiments, is a fully shared-stem probe. In other embodiments, every probe in each channel in which at least 3 different target-probe fluorescence signatures are discriminable is a shared-stem probe, or in other embodiments, is a fully shared-stem probe.

The aforementioned reaction mixtures have, in some embodiments, 6-25 primer sets, per tube. In other embodiments, there are 8-25 primer sets. In still other embodiments, there are 10-25 primer sets. In other embodiments, there are 12-25 primer sets. In other embodiments, there are 13-25 primer sets. In other embodiments, there are at least 6 primer sets. In other embodiments, there are at least 8 primer sets. In other embodiments, there are at least 10 primer sets. In other embodiments, there are at least 12 primer sets. In other embodiments, there are at least 13 primer sets. All the above ranges are inclusive.

Alternatively or in addition, the reaction mixtures have 6-25 probes, per tube. In other embodiments, there are 8-25 probes. In still other embodiments, there are 10-25 probes. In other embodiments, there are 12-25 probes. In other embodiments, there are 13-25 probes. In other embodiments, there are at least 6 probes. In other embodiments, there are at least 8 probes. In other embodiments, there are at least 10 probes. In other embodiments, there are at least 12 probes. In other embodiments, there are at least 13 probes. All the above ranges are inclusive.

In some embodiments, the probes bind to single-stranded polynucleotides in a sequence-specific manner. In more specific embodiments, the probes may be Molecular Beacons probes, which are described inter alia in U.S. Pat. Nos. 5,925,517, 6,037,130, 6,103,476, 6,150,097, 6,461,817 and 7,385,043, the contents of which are incorporated herein by reference.

In yet other embodiments of the aforementioned reaction mixture, there are 6-20 primer sets and 6-20 probes, which fluoresce in 4-7 different channels. In still other embodiments, there are 8-20 primer sets and 8-20 probes, which fluoresce in 4-7 different channels. In other embodiments, there are 10-20 primer sets and 8-20 probes, which fluoresce in 4-7 different channels. In other embodiments, there are 10-15 primer sets and 10-15 probes, which fluoresce in 4-7 different channels. In still other embodiments, there are 12-15 primer sets and 12-15 probes, which fluoresce in 4-7 different channels. In other embodiments, there are at least 12 primer sets and at least 12 probes, which fluoresce in 4-7 different channels. All the above ranges are inclusive.

In yet other embodiments of the aforementioned reaction mixture, there are 6-20 primer sets and 6-20 probes, which fluoresce in at least 5 different channels. In still other embodiments, there are 8-20 primer sets and 8-20 probes, which fluoresce in at least 5 different channels. In other embodiments, there are 10-20 primer sets and 8-20 probes, which fluoresce in at least 5 different channels. In other embodiments, there are 10-15 primer sets and 10-15 probes, which fluoresce in at least 5 different channels. In still other embodiments, there are 12-15 primer sets and 12-15 probes, which fluoresce in at least 5 different channels. In other embodiments, there are at least 12 primer sets and at least 12 probes, which fluoresce in at least 5 different channels. All the above ranges are inclusive.

In yet other embodiments of the aforementioned reaction mixture, there are 6-20 primer sets and 6-20 probes, which fluoresce in 5 different channels. In still other embodiments, there are 8-20 primer sets and 8-20 probes, which fluoresce in 5 different channels. In other embodiments, there are 10-20 primer sets and 8-20 probes, which fluoresce in 5 different channels. In other embodiments, there are 10-15 primer sets and 10-15 probes, which fluoresce in 5 different channels. In still other embodiments, there are 12-15 primer sets and 12-15 probes, which fluoresce in 5 different channels. In other embodiments, there are at least 12 primer sets and at least 12 probes, which fluoresce in 5 different channels. All the above ranges are inclusive.

In certain embodiments of the described methods and compositions, when the probes that fluoresce in one channel are considered, most of all of them fall within a particular range of lengths. This may be the case of multiple channels as well. For example, in some embodiments it is the case, for at least 1 of the channels, that most or all of the probes that fluoresce in that channel have a length of between 19-26 nucleotides inclusive, between 20-26 nucleotides inclusive, or between 21-26 nucleotides inclusive. It is shown herein that this length is advantageous in many of the described methods and compositions. In other embodiments, this length range applies to most of the probes of at least 2 of the channels. In other embodiments, this length range applies to most of the probes of at least 3 of the channels. In other embodiments, this length range applies to most of the probes of at least 4 of the channels. In still other embodiments, this length range applies to most of the probes of at least 5 of the channels. In still other embodiments, this length range applies to most of the probes of each of at least the orange, red, and crimson channels.

In still other embodiments, it is the case, for at least one of the channels, that each probe that fluoresces in that channel has a length of between 19-26 nucleotides inclusive, between 20-26 nucleotides inclusive, or between 21-26 nucleotides inclusive. In other embodiments, this is the case for at least two channels. In other embodiments, this is the case for at least three channels. In other embodiments, this is the case for at least four channels. In other embodiments, this is the case for at least five channels. In other embodiments, this is the case for all the channels. In other embodiments, this is the case for the orange, red, and crimson channels.

In other embodiments, the majority of probes in the reaction mixture have a length of between 19-26 nucleotides inclusive, between 20-26 nucleotides inclusive, or between 21-26 nucleotides inclusive.

In more specific embodiments, when the probes in the reaction mixture that have a length of between 21-26 nucleotides inclusive are considered as a group, the majority of these probes are shared-stem probes, or in another embodiment fully shared-stem probes. As provided herein, shared-stem probes exhibit mismatch tolerance, thereby enabling detection of targets with sequence variability. A non-limiting example of this is 28S-CA-PB, which detects the 28S gene for both Aspergillus and Candida, despite several mismatches to the Candida gene.

In other embodiments, for each channel in which most or all of the probes of said channel have a length of between 21-26 nucleotides inclusive, at least one probe is at least a partial shared-stem probe, or in another embodiment a fully shared-stem probe, or in another embodiment a double, shared-stem probe, or in another embodiment a double, fully shared-stem probe.

In yet other embodiments, most probes in at least one channel either fall within the length range of 19-26 nucleotides inclusive or within 34-55 nucleotides inclusive. Thus, the particular channel could have probes that fall within one or both of these ranges, provided that the sum of the probes within these ranges constitutes the majority of probes in that channel. In other embodiments, most probes in the channel fall between 20-26 nucleotides inclusive or 34-55 nucleotides inclusive; in yet other embodiments between 21-26 nucleotides inclusive or 34-55 nucleotides inclusive. In other embodiments, this is the case for at least 2 channels. In other embodiments, this is the case for at least 3 channels. In other embodiments, this is the case for at least 4 channels. In other embodiments, this is the case for at least 5 channels. In other embodiments, this is the case for all the channels. In other embodiments, this is the case for the orange, red, and crimson channels.

In other embodiments, the majority of probes in the reaction mixture either fall within the length range of 19-26 nucleotides inclusive, in other embodiments between 20-26 nucleotides inclusive, or in other embodiments between 21-26 nucleotides inclusive.

In other embodiments, it is the case, for each channel in which 2 or more different target-probe fluorescence signatures are discriminable, that the majority of probes have a length of between 21-26 nucleotides inclusive. In other embodiments, it is the case, for each channel in which 2 or more different target-probe fluorescence signatures are discriminable, that the majority of probes either fall within the length range of 21-26 nucleotides inclusive or 34-55 nucleotides inclusive.

In other embodiments, the majority of probes in the orange, red, and crimson channels, taken together, have a length of between 19-26 nucleotides inclusive, in other embodiments between 20-26 nucleotides inclusive, or in other embodiments between 21-26 nucleotides inclusive. In other embodiments, the majority of probes in the orange, red, and crimson channels, taken together, either fall within the length range of 21-26 nucleotides inclusive or 34-55 nucleotides inclusive.

In other embodiments, the majority of probes in the reaction mixture, exclusive of the green and yellow channels, have a length of between 19-26 nucleotides inclusive, in other embodiments between 20-26 nucleotides inclusive, or in other embodiments between 21-26 nucleotides inclusive. In other embodiments, the majority of probes in the reaction mixture, exclusive of the green and yellow channels, either fall within the length range of 21-26 nucleotides inclusive or 34-55 nucleotides inclusive.

Also provided herein is a reaction mixture, comprising: (a) a nucleotide-containing test sample (e.g. a DNA extract of a blood sample from a human); (b) 6 or more primer sets, wherein at least the majority of the primer sets is asymmetric; and (c) 6 or more probes, which fluoresce in 4 or more different channels, wherein:
i. each of the probes binds to a polynucleotide selected from (i) a PCR product of a target amplified by one or more of the primer sets, typically the excess strand in the case of asymmetric amplification; and (ii) a control polynucleotide, whereupon fluorescence of the probe is activated; and
ii. in at least 1 of the channels, a plurality of (at least 2) different target-probe fluorescence signatures are discriminable;
iii. where, for each channel in which at least two, or in other embodiments three, different target-probe fluorescence signatures are discriminable, the following two statements are true:
   a. At least the majority of the probes that fluoresce in the channel, or in other embodiments each probe in the channel, has a length of between 21-26 nucleotides inclusive or 34-55 nucleotides inclusive. Thus, the particular channel could have probes that fall within one or both of these ranges, provided that the sum of the probes within these ranges constitutes the majority of probes in that channel; and
   b. At least one probe that fluoresces in the channel is a shared-stem probe.

In other embodiments, for each channel in which at least two, or in other embodiments three, different target-probe fluorescence signatures are discriminable, at least the majority of the probes in said channel is a shared-stem probe.

Typically, the aforementioned reaction mixture is indicated for amplification and detection in a single reaction tube. In other embodiments, the mixture is provided in a single reaction tube.

Provided herein, in yet other embodiments, is a reaction mixture, comprising: (a) a nucleotide-containing test sample (e.g. a DNA extract of a blood sample from a human); (b) 6 or more primer sets, wherein at least the majority of the primer sets is asymmetric; and (c) 6 or more probes, which fluoresce in 4 or more different channels, wherein:
i. each of the probes binds to a polynucleotide selected from (i) a PCR product of a target amplified by one or more of the primer sets, typically the excess strand in the case of asymmetric amplification; and (ii) a control polynucleotide, whereupon fluorescence of the probe is activated; and
ii. in at least 1 of the channels, a plurality of (at least two) different target-probe fluorescence signatures are discriminable;
iii. where, for each channel in which at least two, or in other embodiments three, different target-probe fluorescence signatures are discriminable, at least one probe that fluoresces in the channel is a shared-stem probe.

In other embodiments of the aforementioned mixtures, for each channel in which at least two, or in other embodiments three, different target-probe fluorescence signatures are discriminable, at least the majority of the probes are shared-stem probes.

Typically, the aforementioned reaction mixtures are indicated for amplification and detection in a single reaction tube. In other embodiments, the mixture is provided in a single reaction tube.

Provided herein, in still other embodiments, is a reaction mixture, comprising: (a) a nucleotide-containing test sample (e.g. a DNA extract of a blood sample from a human); (b) 6 or more primer sets, wherein at least the majority of the primer sets is asymmetric; and (c) 6 or more probes, which fluoresce in 4 or more different channels, wherein:
i. each of the probes binds to a polynucleotide selected from (i) a PCR product of a target amplified by one or more of the primer sets, typically the excess strand in the case of asymmetric amplification; and (ii) a control polynucleotide, whereupon fluorescence of the probe is activated; and
ii. in at least 1 of the channels, a plurality of (at least 2) different target-probe fluorescence signatures are discriminable;
iii. where, for each channel in which at least two, or in other embodiments three, different target-probe fluorescence signatures are discriminable, the ΔT_{M} is between 6-13° C. inclusive.

In more specific embodiments of the aforementioned reaction mixture, Statement (A) below, in in other embodiments Statement (B), or in other embodiments both Statement (A) and Statement (B), is also true of each channel in which at least two, or in other embodiments three, different target-probe fluorescence signatures are discriminable:
Statement A: At least the majority of the probes that fluoresce in the channel, or in other embodiments each probe in the channel, has a length of between 21-26 nucleotides inclusive or 34-55 nucleotides inclusive; and
Statement B: At least one probe that fluoresces in the channel is a shared-stem probe.

In other embodiments, for each channel in which at least two, or in other embodiments three, different target-probe fluorescence signatures are discriminable, at least the majority of the probes in said channel is a shared-stem probe. Alternatively or in addition, at least the majority of the primer sets in the reaction mixture are hot-start primers. In other embodiments, all the primers in the reaction mixture are hot-start primers.

Typically, the aforementioned reaction mixture is indicated for amplification and detection in a single reaction tube. In other embodiments, the mixture is provided in a single reaction tube.

Targets

Those skilled in the art will appreciate, in light of the present disclosure, that a variety of targets are suitable for the described compositions and methods. In some embodiments, the targets are selected from known polynucleotides found in a pathogen, and thus suspected to be present in the test sample, and internal control polynucleotides. In other embodiments, polynucleotides characteristic of a known human pathogen are included in the list of targets. In more specific embodiments, the pathogen is in each case selected from a bacterium and a fungus; or in other embodiments from a bacterium, a fungus, and a parasitic protozoan; or in other embodiments, a bacterium, a fungus, and a mold; or in other embodiments, a bacterium, a fungus, a parasitic protozoan, and a mold. Malaria is a non-limiting example of a parasitic protozoan that causes disease in humans.

Reference herein to a polynucleotide sequence “characteristic of” a target pathogen, or reference to a “marker” polynucleotide, indicates that the sequence can be used to distinguish the target pathogen from other types of pathogens. Those skilled in the art will appreciate that, in various embodiments, depending on the purpose of the assay and the medical scenario, the polynucleotide sequence may be unique to a particular pathogen strain, a particular pathogen species, or a particular pathogen genus, or a particular subset of a pathogen genus, and may be carried on a plasmid or integrated into the genome. Non-limiting embodiments of pathogen-specific polynucleotides and general pathogen class marker polynucleotides that may be used in the described method and compositions are nuc and spa (immunoglobulin G binding protein A) of S. aureus; tuf of non-SA staphylococcus; the “SPN9802” sequence of Streptococcus pneumoniae; the gene encoding bacterial 16S rRNA; oprI of Pseudomonas; emm for beta-hemolytic Streptococcus; rpob of Acinetobacter; and for fungi, L1A1, and the genes encoding the 18S and 28S ribosomal RNA (rRNA). Non-limiting embodiments of antibiotic-resistance polynucleotides are mecA, mecC, vanA, vanB, SHV, CTXM-14, CTXM-15, IMP, KPC, GES, OXA-48, vim, and NDM. Additional non-limiting examples of pathogen-specific polynucleotide sequences and primers for amplifying same include Sa442 femB of S. aureus and eae (encoding Intimin Adherence Protein) of E. coli (Gene ID: 960862, updated on 26-Aug.-2013; and/or ATCC #700728), as well as markers described both herein and in US Pat. App. No. 2009/0081663.

In addition to polynucleotide sequences characteristic of a target pathogen, the list of targets also includes, in some embodiments, an antibiotic-resistance gene or polynucleotide sequence. While certain antibiotic-resistance genes or sequences may be particular to a particular pathogen species or a particular genus, others may be found in a variety of pathogen species. As a non-limiting example, the metallo-β-lactamases, serine-β-lactamases, and extended-spectrum-β-lactamases (ESBL's) tend to be found in Enterobacteriaceae (Enterobacteria). In some embodiments of the described methods and compositions, a positive result for one of the aforementioned β-lactamases indicates the presence of Enterobacteria; thus, there is no need to detect separate Enterobacteria marker polynucleotide. In some embodiment, marker polynucleotides for other major types of pathogenic gram-negative bacteria, for example Pseudomonas and/or Acinetobacter, are detected.

In more specific embodiments, the list of targets comprises at least one marker polynucleotide of a pathogenic gram-positive bacterium and at least polynucleotide associated with an antibiotic resistance in said gram-positive bacterium. In other embodiments, the list of targets may comprise at least one marker polynucleotide of a pathogenic gram-negative bacterium and at least polynucleotide associated with an antibiotic resistance in said gram-negative bacterium. Additionally, the list of targets may comprise at least one marker polynucleotide of a pathogenic fungus.

In other embodiments, the described mixtures and methods utilize an intact Archaeon as the specimen-processing control. For example, Methanothermobacter has a cell wall (like other Archaea) and has a 16S gene that differs from that of bacteria such that it is not recognized by the 16S probe used herein. Thus, amplification of this or another Archaeon polynucleotide can serve to verify that the bacterial cells have been lysed, and compounds that inhibit PCR have been removed. In other embodiments, the specimen-processing control is added to a tube that is processed in parallel with the test samples.

In other embodiments is provided a PCR reaction mixture, comprising
a DNA polymerase;
dNTPs;
magnesium ions—these are, in some embodiments, supplied separately from the dried PCR mixture;
one or more salts—which may be, in non-limiting embodiments, potassium chloride (KCl);
a pH buffer; and
an intact Archaeon.

In some embodiments, the aforementioned reaction mixture further comprises one or more of: a thermophilic RNAse, BSA, and sucrose. In some more specific embodiments, the primers are ribo-primers. In still other embodiments, probes are included as well. In some more specific embodiments, the primers and probes are designed to amplify a set of GP targets described herein, or in other embodiments, a set of GN targets described herein.

In still other embodiments is provided a method for detecting the presence of a target polynucleotide in a test sample, comprising the steps of: (a) thermocycling a reaction mixture comprising an intact Archaeon, while periodically measuring fluorescence at each of the channels. In other embodiments, the method further comprises the steps of (b) subjecting the product of step (a) to a controlled heating or controlled cooling, while periodically measuring fluorescence at each of the channels; and (c) for each channel in which at least 2 different target-probe fluorescence signatures are discriminable, identifying the fluorescence signature that is present (provided that a signal is present). In still other embodiments, the sample processing is automated.

In other embodiments, the described mixtures and methods utilize an internal control or other plasmid isolated from a non-bacterial source, such as yeast, in order to prevent false-positives from trace amounts of bacterial DNA, due to the high sensitivity of the assays.

Generating Different, Discriminable Fluorescence Signatures in a Single Channel

As provided herein, different, discriminable target-probe fluorescence signatures may be generated (a) using different probes that fluoresce in the same channel, and that bind to different single-stranded amplification products, each with a unique hybrid T_{M} and/or a unique hybrid melting curve. Different signatures may also be generated by (b) using a single probe that interacts with 2 or more known target sequences, each with a unique hybrid T_{M} and/or a unique hybrid melting curve. The different sequences may be on entirely different loci or, in other embodiments, in variations of a single locus. In some embodiments of (b), a probe utilized in the methods and compositions described herein may be engineered to be mismatch tolerant, such that sequence variations are recognized, but with different hybrid fluorescence signatures, so they can be distinguished. In still other embodiments, a probe is engineered to be mismatch intolerant, in order that only certain variant(s) of a target sequence is detected. An example of this is SHV-PB, which detects many known 2be and 2br SHV variants, such as SHV-2, but not 2b SHV variants, such as SHV-1.

In still other embodiments, a combination of (a) and (b) from the previous paragraph is utilized. In other embodiments, (a) is the case in at least two channels that have discriminable target-probe fluorescence signatures. In yet other embodiments, (b) is the case in at least one channel that has discriminable target-probe fluorescence signatures. In yet other embodiments, (a) is the case in at least two channels that have discriminable target-probe fluorescence signatures, and (b) is the case in at least one channel that has discriminable target-probe fluorescence signatures.

Ranges and Values for Delta T_{M}

In other embodiments, the ΔT_{M} values of the probes of the methods and compositions described herein are within particular ranges. In some embodiments, the ΔTM values of at least most of the probes in the reaction mixture are from 5-14° C. inclusive, from 6-13° C. inclusive, or from 7-12° C. inclusive. In other embodiments, the ΔTM values of at least most of the probes in the channels for which two, or in another embodiments three, different target-probe fluorescence signatures are discriminable, when said channels are considered together, are from 5-14° C. inclusive, from 6-13° C. inclusive, or from 7-12° C. inclusive. In still other embodiments, the ΔTM values of at least most of the probes in the orange, red, and crimson channels, when said channels are considered together, are from 5-14° C. inclusive, from 6-13° C. inclusive, or from 7-12° C. inclusive. In this latter embodiment, it is, in further embodiments, also the case that the ΔTM values of at least most of the probes in the yellow and green channels are between 7-14° C. inclusive; or in other embodiments between 8-13° C. inclusive; or in other embodiments between 9-13° C. inclusive; or in other embodiments between 8-12° C. inclusive; or in other embodiments between 9-12° C. inclusive.

In other embodiments, for cases in which more than one target sequence is desired to be detected, the ΔT_{M} value for each desired hybrid is from 5-14° C. inclusive. in still other embodiments, for cases in which more than one target sequence is known, and one or more is desired to be detected, while other sequence(s) are not desired to be detected, the ΔTM value for the desired hybrid(s) is from 5-14° C. inclusive, while the ΔTM value for the undesired hybrid(s) is higher than 15° C., or in other embodiments higher than 18° C. In other embodiments of this scenario, the ΔTM value for the desired hybrid(s) is from 6-13° C. inclusive, while the ΔTM value for the undesired hybrid(s) is higher than 15° C., or in other embodiments higher than 18° C. In still other embodiments of this scenario, the ΔTM value for the desired hybrid(s) is from 7-12° C. inclusive, while the ΔTM value for the undesired hybrid(s) is higher than 15° C., or in other embodiments higher than 18° C.

In yet other embodiments, for each probe in the reaction mixture, the ΔT_{M} value is between 1-17° C. inclusive; in other embodiments between 2-16° C. inclusive; in other embodiments between 3-15° C. inclusive; in other embodiments between 4-14° C. inclusive; or in other embodiments between 5-14° C. inclusive. In other embodiments, the ΔTM values of all the probes in the channels for which two, or in another embodiments three, different target-probe fluorescence signatures are discriminable, are between 1-17° C. inclusive; in other embodiments between 2-16° C. inclusive; in other embodiments between 3-15° C. inclusive; in other embodiments between 4-14° C. inclusive; or in other embodiments between 5-14° C. inclusive. In still other embodiments, the ΔTM values of all the probes in the orange, red, and crimson channels, are between 1-17° C. inclusive; in other embodiments between 2-16° C. inclusive; in other embodiments between 3-15° C. inclusive; in other embodiments between 4-14° C. inclusive; or in other embodiments between 5-14° C. inclusive. In this latter embodiment, it is, in further embodiments, also the case that the ΔTM values of all the probes in the yellow and green channels are between 7-17° C. inclusive; or in other embodiments between 8-16° C. inclusive; or in other embodiments between 9-15° C. inclusive; or in other embodiments between 10-15° C. inclusive; or in other embodiments between 10-14° C. inclusive.

Ranges and Values for Hybrid T_{M}

In still other embodiments, the hybrid T_{M} values of the described probes with the sequence desired to be detected, or if more than one sequence is desired to be detected, with all the desired sequences, are within particular ranges. In some embodiments, the hybrid T_{M} values of at least most of the probes in the reaction mixture are between 58-72° C. inclusive, in other embodiments between 57-73° C., in other embodiments between 56-74° C. inclusive, in other embodiments between 58-71° C. inclusive, in other embodiments between 58-70° C. inclusive, in other embodiments between 58-73° C. inclusive, in other embodiments between 58-74° C. inclusive, in other embodiments between 58-75° C. inclusive, in other embodiments between 58-76° C. inclusive. In other embodiments, the hybrid T_{M} values of at least most of the probes in the channels for which two, or in another embodiments three, different target-probe fluorescence signatures are discriminable, where these channels are considered together, are between 58-72° C. inclusive, in other embodiments between 57-73° C., in other embodiments between 56-74° C. inclusive, in other embodiments between 58-71° C. inclusive, in other embodiments between 58-70° C. inclusive, in other embodiments between 58-69° C. inclusive. In other embodiments, the hybrid T_{M} values of at least most of the probes in the orange, red, and crimson channels, where these channels are considered together, are between 58-72° C. inclusive, in other embodiments between 57-73° C., in other embodiments between 56-74° C. inclusive, in other embodiments between 58-71° C. inclusive, in other embodiments between 58-70° C. inclusive, in other embodiments between 58-69° C. inclusive.

In still other embodiments, for example if fluorescence is not monitored during amplification, the hybrid T_{M} values of at least most of the probes in the reaction mixture can be between 40-72° C. inclusive, in other embodiments between 39-73° C., in other embodiments from 41-74° C. inclusive, in other embodiments between 40-71° C. inclusive, in other embodiments from 40-70° C. inclusive, in other embodiments between 40-73° C. inclusive, in other embodiments between 40-74° C. inclusive, in other embodiments between 40-75° C. inclusive, in other embodiments between 40-76° C. inclusive.

In yet other embodiments, the hybrid T_{M} values of all the probes in the reaction mixture are between 56-76° C. inclusive, in other embodiments between 57-75° C., in other embodiments between 58-74° C. inclusive, in other embodiments between 57-76° C. inclusive, in other embodiments between 56-75° C. inclusive. In other embodiments, the hybrid T_{M} values of all the probes in the channels for which two, or in another embodiments three, different target-probe fluorescence signatures are discriminable are between 56-76° C. inclusive, in other embodiments between 57-75° C., in other embodiments between 58-74° C. inclusive, in other embodiments between 57-76° C. inclusive, in other embodiments between 56-75° C. inclusive. In other embodiments, the hybrid T_{M} values of all the probes in the orange, red, and crimson channels are between 56-76° C. inclusive, in other embodiments between 57-75° C., in other embodiments between 58-74° C. inclusive, in other embodiments between 57-76° C. inclusive, in other embodiments between 56-75° C. inclusive.

Ranges and Values for Internal TM

In still other embodiments, the internal T_{M} values of the described probes with the sequence desired to be detected, or if more than one sequence is desired to be detected, with all the desired sequences, are within particular ranges. In some embodiments, for most or all of the probes in the reaction mixture, the internal T_{M} is between 65-82° C. inclusive, in other embodiment between 64-83° C. inclusive, in other embodiment between 66-81° C. inclusive, in other embodiment between 67-80° C. inclusive, in other embodiment between 68-79° C. inclusive, in other embodiment between 68-78° C. inclusive. In other embodiments, for most or all of the probes in the channels for which two, or in another embodiments three, different target-probe fluorescence signatures are discriminable, where these channels are considered together, the internal T_{M} is between 65-82° C. inclusive, in other embodiment between 64-83° C. inclusive, in other embodiment between 66-81° C. inclusive, in other embodiment between 67-80° C. inclusive, in other embodiment between 68-79° C. inclusive, in other embodiment between 68-78° C. inclusive. In other embodiments, for most or all of the probes in the orange, red, and crimson channels, where these channels are considered together, the internal T_{M} is between 65-82° C. inclusive, in other embodiment between 64-83° C. inclusive, in other embodiment between 66-81° C. inclusive, in other embodiment between 67-80° C. inclusive, in other embodiment between 68-79° C. inclusive, in other embodiment between 68-78° C. inclusive.

In still embodiments, for all the probes in the reaction mixture, the internal T_{M} is between 63-86° C. inclusive, in other embodiment between 63-85° C. inclusive, in other embodiment between 64-85° C. inclusive, in other embodiment between 64-84° C. inclusive. In other embodiments, for all the probes in the channels for which two, or in other embodiments three, different target-probe fluorescence signatures are discriminable, the internal T_{M} is between 63-86° C. inclusive, in other embodiment between 63-85° C. inclusive, in other embodiment between 64-85° C. inclusive, in other embodiment between 64-84° C. inclusive. In other embodiments, for all the probes in the orange, red, and crimson channels, the internal T_{M} is between 63-86° C. inclusive, in other embodiment between 63-85° C. inclusive, in other embodiment between 64-85° C. inclusive, in other embodiment between 64-84° C. inclusive.

In certain embodiments, at least 3 different target-probe fluorescence signatures are discriminable in each of the orange, red, and crimson channels. In more specific embodiments, at least 3 different target-probe fluorescence signatures are discriminable in each of the orange, red, and crimson channels, and the yellow and green channels are also utilized, but without an attempt to distinguish different target-probe fluorescence signatures in these channels. In other embodiments, all the information necessary from the yellow and green channels is obtained from the amplification signal, for example if there is only one probe in these channel, or in other embodiment, if one or more of these channels has multiple probes, but there is no medical difference between the different targets detected in that channel. In still other embodiments, at least 3 different target-probe fluorescence signatures are discriminable in each of the orange, red, and crimson channels, and at least 2 different target-probe fluorescence signatures are discriminable in each of the yellow and green channels.

Other Components

In yet other embodiments, the described reaction mixture further comprises one or more of the following:
a DNA polymerase (which may be, in non-limiting embodiments, a thermophilic polymerase such as taq [Thermus aquaticus] polymerase or pfu [Pyrococcus furiosus] polymerase);
deoxynucleoside triphosphates (dNTPs);
magnesium ions—these are, in some embodiments, supplied separately from the dried PCR mixture;
one or more salts (which may be, in non-limiting embodiments, potassium chloride [KCl]);
a pH buffer; and
any one or more of: a thermophilic RNAse (which may be an RNAse H and/or a thermophilic RNAse, non-limiting examples of which are or RNAse H2, for example Pyrococcus abyssi Ribonuclease H2 endonuclease), BSA and sucrose. In more specific embodiments, the RNAse H2 enzyme is thermostable and thermophilic.

Non-limiting embodiments of a pH buffer are Tris-pH buffers, having a slightly alkaline pH, for example between 7.5-9. In some embodiments, the pH is around 8.3.

Some embodiments of the described methods and compositions utilize ribo-primers that are activated using an RNase H2 enzyme, which is, in some embodiments, a thermophilic RNase H2 enzyme. Thermostable RNase H2 enzymes and methods for using same are well known in the art (Haruki et al, Gene Cloning and Characterization of Recombinant RNase HII from a Hyperthermophilic Archaeon. Journal of Bacteriology, December 1998, p. 6207-6214.) An exemplary, non-limiting thermostable RNase H2 enzyme is the P. abyssi Ribonuclease H2 enzyme, which is utilized in the Examples herein. P. abyssi RNaseH2 is a thermo-stable and thermophilic RNaseH enzyme. The RNaseH enzyme binds to regions where a ribonucleotide is bound to a deoxyribonucleotide. Once bound, the enzyme cleaves immediately 5′ of the RNA residue, removing the ribonucleotide and bases 3′ of it, thus leaving a slightly shorter DNA oligonucleotide with a 3′ end that can be extended by polymerase. In various embodiments, the hot-start/thermophilic properties of an RNase H2 enzyme used in conjunction with ribo-primers in the described methods and compositions may be either intrinsic to the enzyme or a result of reversible chemical inactivation or a blocking antibody, as is well known in the art.

Alternatively or in addition, ribo-primers and P. abyssi RNaseH2 are used in conjunction with a hot-start polymerase such as taq polymerase. In more specific embodiments, magnesium ions may be supplied as part of the dried PCR mixture containing taq polymerase.

Methods

In other embodiments, a method is provided for detecting the presence of a target polynucleotide in a test sample, comprising the steps of: (a) thermocycling a reaction mixture described herein, while periodically measuring fluorescence at each of the channels; (b) subjecting the product of step (a) to a controlled heating or controlled cooling, while periodically measuring fluorescence at each of the channels; and (c) for each channel in which at least 2 different target-probe fluorescence signatures are discriminable, identifying the fluorescence signature that is present (provided that a signal is present). In some embodiments, the controlled heating or controlled cooling is performed stepwise, while in other embodiments, it may be gradual. Either alternative is acceptable, provided that the temperature is monitored, and fluorescence is measured at predetermined temperatures. Those skilled in the art will appreciate that the step of thermocycling typically comprises the sub-steps of strand melting, annealing and primer extension. Generally, it is performed repeatedly. In certain embodiments, the step is repeated between 30-55 times.

The terms “controlled heating” and “controlled cooling”, or “controlled melt(ing)” and “controlled anneal(ing)”, as used herein, refer to a predetermined gradual heating to cooling process. In some embodiments, the following parameters are predetermined: The minimum and maximum temperatures (starting and ending temperatures), the increments of temperature change, the rate of change between temperatures (optionally), and the pause time at each temperature. In certain embodiments, there are one or more defined steps before heating or cooling, at which the temperature is held constant. An example of such a program follows below. Those skilled in the art will appreciate in light of the present disclosure that the exact parameters of the controlled heating or controlled cooling are not critical for carrying out the described methods.

Example of Controlled Heating:
Heat to 95 deg for 60 sec
Reduce temperature to 40 deg, hold at 40 deg for 90 sec
Heat to 95 deg at increments of 1 degree, stopping for 5 sec and measuring fluorescence at each step.

Also provided is a method of detecting the presence of a gram-positive bacterium in a test sample and the presence of a polynucleotide sequence associated with antibiotic resistance in a GP bacterium, the method comprising the step of thermocycling a described reaction mixture, while periodically measuring fluorescence at each of the channels. In some embodiments, the method further comprises the steps of: (b) subjecting the product of step (a) to a controlled heating or controlled cooling, while periodically measuring fluorescence at each of the channels; and (c) for each channel in which a signal is present and at least 2 different target-probe fluorescence signatures are discriminable, identifying the fluorescence signature that is present. In some embodiments, the presence of an SA marker indicates the presence of SA in the sample, while the presence of a general Staphylococcus marker in the absence of a SA marker indicates the presence of non-aureus Staphylococcus. Alternatively or in addition, the presence of a general GP bacteria marker, in the absence of a general Staphylococcus marker, a S. pneumoniae marker, and a marker for E. faecium and E. faecalis indicates the presence of a GP bacterium that is other than Staphylococcus, S. pneumoniae, E. faecium, or E. faecalis. Alternatively or in addition, the presence of a marker polynucleotide for a pathogen, e.g. SA, S. pneumoniae, E. faecium, or E. faecalis, together with the presence of an antibiotic-resistance polynucleotide, indicates the presence of both the indicated pathogen and the indicated polynucleotide. On the other hand, the presence of the pathogen marker polynucleotide, in the absence of the antibiotic-resistance polynucleotide, indicates that the pathogen but not the indicated polynucleotide is present.

Also provided is a method of detecting the presence of a gram-positive bacterium and/or a fungus and/or the presence of a polynucleotide sequence associated with antibiotic resistance in a GP bacterium, the method comprising the step of thermocycling a described reaction mixture, while periodically measuring fluorescence at each of the channels. In some embodiments, the method further comprises the steps of: (b) subjecting the product of step (a) to a controlled heating or controlled cooling, while periodically measuring fluorescence at each of the channels; and (c) for each channel in which a signal is present and at least 2 different target-probe fluorescence signatures are discriminable, identifying the fluorescence signature that is present. In some embodiments, the presence of an SA marker indicates the presence of SA in the sample, while the presence of a general Staphylococcus marker in the absence of a SA marker indicates the presence of non-aureus Staphylococcus. Alternatively or in addition, the presence of a general GP bacteria marker, in the absence of a Staphylococcus marker, a S. pneumoniae marker, and a marker for E. faecium and E. faecalis indicates the presence of a GP bacterium that is other than Staphylococcus, S. pneumoniae, E. faecium, or E. faecalis. Alternatively or in addition, the presence of an Aspergillus or Candida marker indicates the presence of Aspergillus or Candida, respectively, while the presence of a general fungal marker in the absence of an Aspergillus or Candida marker indicates the presence of a fungal infection other than Aspergillus or Candida.

Provided in other embodiments is a method of detecting the presence of a gram-negative bacterium in a test sample and the presence of a polynucleotide sequence associated with antibiotic resistance in a GN bacterium, the method comprising the step of incubating a described reaction mixture in a thermocycler machine, while periodically measuring fluorescence at each of the channels. In some embodiments, the method further comprises the steps of: (b) subjecting the product of step (a) to a controlled heating or controlled cooling, while periodically measuring fluorescence at each of the channels; and (c) for each channel in which a signal is present and at least 2 different target-probe fluorescence signatures are discriminable, identifying the fluorescence signature that is present. In some embodiments, the presence of a GN bacteria marker in the absence of a polynucleotide encoding a metallo-β-lactamase sequence, a serine-β-lactamase nucleotide sequence, a subgroup 2be β-lactamase, or a subgroup 2br β-lactamase indicates the presence of a GN bacteria that does not contain one of the listed β-lactamases. Alternatively or in addition, the presence of a general GN bacteria marker, in the absence of an Acinetobacter marker indicates the presence of a GN bacterium that is other than Acinetobacter.

In other embodiments, a method is provided for confirming and determining the cause of a suspected case of sepsis, the method comprising the steps of: (a) thermocycling a described GP bacteria reaction mixture, while periodically measuring fluorescence at each of the channels; and (b) using a logic matrix to identify the pathogenic agents and antibiotic-resistance polynucleotides present in the test sample. In some embodiments, the method further comprises the steps of: (c) subjecting the product of step (a) to a controlled heating or controlled cooling, while periodically measuring fluorescence at each of the channels; and (d) for each channel in which a signal is present and at least 2 different target-probe fluorescence signatures are discriminable, identifying the fluorescence signature that is present, wherein the aforementioned logic matrix may be applied to the results of step (a) and/or steps (c-d).

Those skilled in the art will appreciate that logic matrices that may be used in the described methods and compositions may be derived from the Experimental Details section. For example, the presence of a marker polynucleotide for a pathogen, e.g. SA, S. pneumoniae, E. faecium, or E. faecalis, together with the presence of an antibiotic-resistance polynucleotide, indicates the presence of the indicated pathogen, carrying the indicated polynucleotide. On the other hand, the presence of the pathogen marker polynucleotide, in the absence of the antibiotic-resistance polynucleotide, indicates that the pathogen is not carrying the indicated polynucleotide. As another example, if methicillin resistance is positive, the general Staphylococcus marker is positive, and the SA marker is negative, the result is methicillin-resistant, coagulase-negative Staphylococcus.

In other embodiments, a method is provided for confirming and determining the cause of a suspected case of sepsis, the method comprising the steps of: (a) thermocycling a described GN bacteria reaction mixture, while periodically measuring fluorescence at each of the channels; and (b) using a logic matrix to identify the pathogenic agents and antibiotic-resistance polynucleotides present in the test sample. In some embodiments, the method further comprises the steps of: (c) subjecting the product of step (a) to a controlled heating or controlled cooling, while periodically measuring fluorescence at each of the channels; and (d) for each channel in which a signal is present and at least 2 different target-probe fluorescence signatures are discriminable, identifying the fluorescence signature that is present, wherein the aforementioned logic matrix may be applied to the results of step (a) and/or steps (c-d).

In other embodiments, a method is provided for confirming and determining the cause of a suspected case of sepsis, the method comprising the steps of: (a) thermocycling a described GP+GN bacteria reaction mixture, while periodically measuring fluorescence at each of the channels; and (b) using a logic matrix to identify the pathogenic agents and antibiotic-resistance polynucleotides present in the test sample. In some embodiments, the method further comprises the steps of: (c) subjecting the product of step (a) to a controlled heating or controlled cooling, while periodically measuring fluorescence at each of the channels; and (d) for each channel in which a signal is present and at least 2 different target-probe fluorescence signatures are discriminable, identifying the fluorescence signature that is present, wherein the aforementioned logic matrix may be applied to the results of step (a) and/or steps (c-d).

In other embodiments, a method is provided for confirming and determining the cause of a suspected case of sepsis, the method comprising the steps of: (a) thermocycling a described GP+GN bacteria+fungal reaction mixture, while periodically measuring fluorescence at each of the channels; and (b) using a logic matrix to identify the pathogenic agents and antibiotic-resistance polynucleotides present in the test sample. In some embodiments, the method further comprises the steps of: (c) subjecting the product of step (a) to a controlled heating or controlled cooling, while periodically measuring fluorescence at each of the channels; (d) for each channel in which a signal is present and at least 2 different target-probe fluorescence signatures are discriminable, identifying the fluorescence signature that is present, wherein the aforementioned logic matrix may be applied to the results of step (a) and/or steps (c-d).

Provided, in addition, is a method is provided for confirming and determining the cause of a suspected case of sepsis, the method comprising the steps of:
A. isothermally amplifying a test sample, using a reaction mixture that may be present in a single reaction tube or split into several reaction tubes, comprising a group of primer sets that amplify a set of targets suspected of being present in the sample, where the targets comprise: at least one marker polynucleotide of a gram-positive bacteria; and at least one antibiotic-resistance polynucleotide; and
B. using a logic matrix to identify the pathogenic agents and antibiotic-resistance polynucleotides present in the test sample.

In certain embodiments, a helicase enzyme is also present in the aforementioned reaction mixture. In other embodiments, at least the majority, in other embodiments all, of the aforementioned primer sets are ribo-primers, and the reaction mixture further comprises an RNAse H2 enzyme. In other embodiments, the GP marker polynucleotides comprise at least one of: an SA marker; an Enterococcus marker; and an alpha-hemolytic Streptococcus marker (non-limiting embodiments of which are S. pneumoniae marker). Alternatively or in addition, the antibiotic-resistance polynucleotides comprise at least one of: a vancomycin-resistance polynucleotide and a methicillin-resistance polynucleotide. In more specific embodiments, the GP marker polynucleotides comprise an SA marker, a marker for E. faecium and E. faecalis, and an S. pneumoniae marker; and the antibiotic-resistance polynucleotides comprise a vancomycin-resistance polynucleotide and a methicillin-resistance polynucleotide. In other embodiments, other embodiments mentioned herein for GP reaction mixtures, or in other embodiments GP bacteria+fungal reaction mixtures, may apply to the reaction mixture.

Provided, in addition, is a method is provided for confirming and determining the cause of a suspected case of sepsis, the method comprising the steps of:
A. isothermally amplifying a test sample, using a reaction mixture that may be present in a single reaction tube or split into several reaction tubes, comprising a group of primer sets that amplify a set of targets suspected of being present in the sample, where the targets comprise: at least one marker polynucleotide of a gram-negative bacteria; and at least one antibiotic-resistance polynucleotide; and
B. using a logic matrix to identify the pathogenic agents and antibiotic-resistance polynucleotides present in the test sample.

In certain embodiments, a helicase enzyme is also present in the aforementioned reaction mixture. In other embodiments, at least the majority, in other embodiments all, of the aforementioned primer sets are ribo-primers, and the reaction mixture further comprises an RNAse H2 enzyme. In still other embodiments, the GN marker polynucleotide is a general GN marker polynucleotide. Alternatively or in addition, the antibiotic-resistance polynucleotides comprise at least one of: a metallo-β-lactamase sequence, a serine-β-lactamase nucleotide sequence, a subgroup 2be β-lactamase, and a subgroup 2br β-lactamase. In more specific embodiments, the GN marker polynucleotide is a general GN marker polynucleotide; and the antibiotic-resistance polynucleotides comprise a metallo-β-lactamase sequence, a serine-β-lactamase nucleotide sequence, a subgroup 2be β-lactamase, and a subgroup 2br β-lactamase. In other embodiments, other embodiments mentioned herein for GP reaction mixtures, or in other embodiments GP bacteria+fungal reaction mixtures, may apply to the reaction mixture.

In yet other embodiments is provided a method for confirming and determining the cause of a suspected case of sepsis, the method comprising the steps of isothermally amplifying the aforementioned GP and GN reaction mixtures, or in other embodiments GP and GN bacteria+fungal reaction mixtures.

In yet other embodiments, the reaction mixture used in a described method further comprises one or more of the following:
a DNA polymerase (which may be, in non-limiting embodiments, a thermophilic polymerase such as taq polymerase or pfu polymerase);
deoxynucleoside triphosphates (dNTPs);
magnesium ions;
one or more salts (which may be, in non-limiting embodiments, potassium chloride [KCl]);
a pH buffer; and
any one or more of: a thermophilic RNAse (which may be an RNAse H and/or a thermophilic RNAse, non-limiting examples of which are or RNAse H2, for example Pyrococcus abyssi Ribonuclease H2 endonuclease), BSA, and sucrose. In more specific embodiments, the RNAse H2 enzyme is thermostable and thermophilic.

In some embodiments, the described methods further comprise the previous step of processing the sample to purify the DNA present therein, or to enrich the sample in DNA. In the case of a clinical sample (for example, a blood sample or a stool sample), which contains human DNA and is suspected of containing pathogen DNA as well, pathogen DNA is enriched. In more specific embodiments, the processing steps following the withdrawal of the sample from the subject may be automated. Alternatively or in addition, the steps may include selective removal of higher eukaryotic cells, lysis of pathogen cells, and selective removal of non-nucleotide molecules from the sample. In some embodiments, if the magnesium ions are desired to be added separately from the other reaction components, the magnesium ions may be added to the sample, for example at the end of the sample preparation, and then sample can be transferred to the PCR reaction tube.

Those skilled in the art will appreciate that, in certain embodiments of the described methods, fluorescence is periodically and quantitatively measured during the thermocycling and/or heating or cooling steps, such as is routinely performed by devices such as a RotorGene™ 6000 and RotorGene™ Q PCR instruments. In some embodiments, fluorescence is measured after each cycle of the amplification. Alternatively or in addition, fluorescence is measured after each step of the stepwise heating or cooling, or in other embodiments at predetermined temperatures of stepwise heating or cooling.

In some embodiments of the described methods, the appearance of fluorescence in a channel significantly deviating from a negative-control reference standard indicates the presence in the test sample of at least one target detected in that channel, that is, at least one target whose corresponding probe fluoresces in that channel.

Alternatively or in addition, the fluorescence signature is used to indicate which target (or targets) has been amplified, in the case of a positive signal emitting from a channel in which at least 2 different target-probe fluorescence signatures are discriminable.

In yet other embodiments, the aforementioned step of identifying the fluorescence signature that is present includes the following sub-steps: (a) subtracting the fluorescence value of a no-template control from the fluorescence value of the reaction mixture at each timepoint; and (b)
comparing the temporal pattern of the differences obtained in sub-step (a) to a reference standard.

Variations of the Described Methods and Compositions

In other embodiments, the amplification step in the methods and compositions described herein is sufficient to produce actionable results. Thus, fluorescence is determined only during the amplification step, and no controlled melt or annealing of the final PCR product need be performed. In certain, more specific embodiments, the primer sets may be symmetric primer sets. In still other embodiments, the amplification step is sufficient to produce actionable results for some channels, while fluorescence is measured during a controlled melt or annealing in the other channels. In still other embodiments, a first readout is produced following the amplification step, and a second readout is produced following the controlled melt or annealing step. In certain embodiments, at least for certain pathogens, the first readout may be sufficient for the physician to decide which antibiotic should be administered to the patient. Alternatively or in addition, if the first readout is unclear, the second readout will supply the missing information. In still other embodiments, the second readout confirms the result suggested by the first readout. In yet other embodiments, the first readout is sufficient for the physician to decide which antibiotic should be administered, while the second readout provides information desired by epidemiologists, such as the particular metallo-β-lactamase or the particular serine-β-lactamase carried by the patient.

In other embodiments of the methods and compositions described herein, a DNA intercalating dye that binds with little or no sequence specificity, such as SYBR® Green, is used to detect the PCR products. In these embodiments, real-time fluorescence measurement may, or in other embodiments may not, be performed. Alternatively or in addition, a controlled melt or annealing step is performed, and the hybrid fluorescence signature is used, in some embodiments in conjunction with the amplification fluorescence data, to identify the polynucleotide that has been amplified.

In yet other embodiments, a blue channel (e.g. using a probe labeled with Biosearch Blue™ of Biosearch Technologies, having a peak emission at 447 nm, is used instead of the green channel, or in other embodiments instead of the yellow channel. In still other embodiments, the blue channel is utilized in addition to the green and yellow channels.

Also provided herein is a method for detecting gram positive bacteria in a sample, wherein the blood sample contains one or a mixture of bacterial strains or species, the method comprising the steps of: (a) providing primers targeting gram positive specific bacterial strains and species; (b) combining primers into a reaction mixture; and (c) performing an amplification reaction with the reaction mixture, wherein the presence of one or more gram positive bacteria is identified by a logic matrix of the amplification products. The sample may, in various embodiments, be whole blood, plasma, serum, blood bank, neonatal or separated blood, human or veterinary; or may be a blood culture, human or veterinary. In some embodiments, the amplification reaction is a real-time polymerase chain reaction (PCR), in some embodiments using the activation enzyme taq polymerase. Alternatively or in addition, an RNaseH is utilized, for example an RNaseH2.

In still other embodiments, the aforementioned method uses an isothermal amplification reaction, for example further utilizing an RNaseH2.

In some embodiments of this assay, the gram positive bacterial strain or species is identified by amplifying one or more genetic targets, for example by target amplification cutoffs or in other embodiments by amplification curve analysis, by amplification curve comparison, by melting temperature analysis, or by melting temperature comparison. Such determinations may be made manually by an operator, or in other embodiments by an Instrument comprising computer software engineered to determine the gram positive bacterial strain or species.

Also provided is a method for detecting gram negative bacteria in a sample, wherein the blood sample contains one or a mixture of bacterial species, the method comprising: (a) providing primers targeting gram negative specific bacterial strains and species; (b) combining primers into a reaction mixture; and (c) performing an amplification reaction with the reaction mixture, wherein the presence of one or more gram negative bacteria is identified by a logic matrix of the amplification products. The sample may, in various embodiments, be whole blood, plasma, serum, blood bank, neonatal or separated blood, human or veterinary; or may be a blood culture, human or veterinary. In some embodiments, the amplification reaction is a real-time polymerase chain reaction (PCR), in some embodiments using the activation enzyme taq polymerase. Alternatively or in addition, an RNaseH is utilized, for example an RNaseH2.

In still other embodiments, the aforementioned method uses an isothermal amplification reaction, for example further utilizing an RNaseH2.

In some embodiments of this assay, the gram negative bacterial strain or species is identified by amplifying one or more genetic targets, for example by target amplification cutoffs or in other embodiments by amplification curve analysis, by amplification curve comparison, by melting temperature analysis, or by melting temperature comparison. Such determinations may be made manually by an operator, or in other embodiments by an Instrument comprising computer software engineered to determine the gram negative bacterial strain or species.

Also provided is a kit for detecting gram positive and gram negative bacteria in a sample, the kit comprising one or more primer targeting gram positive specific bacterial strains and species and gram negative specific bacterial strains and species. In some embodiments, the kit further comprises sample preparation materials for cell lysis for whole blood, or in other embodiments for lysis for separated blood, or in other embodiments for lysis of cells in blood culture. In other embodiments, one or more of the following components are included: dNTPs, an activating enzyme, and a buffer.

Also provided is a kit for detecting virus in a blood sample, the kit comprising one or more primer pairs targeting viral strains and species. In some embodiments, the kit further comprises sample preparation materials for cell lysis for whole blood, or in other embodiments for lysis for separated blood, or in other embodiments for lysis of cells in blood culture. In other embodiments, one or more of the following components are included: dNTPs, an activating enzyme, and a buffer.

Also provided is a kit for detecting fungus in a blood sample, the kit comprising one or more primer pairs targeting fungal strains and species. In some embodiments, the kit further comprises sample preparation materials for cell lysis for whole blood, or in other embodiments for lysis for separated blood, or in other embodiments for lysis of cells in blood culture. In other embodiments, one or more of the following components are included: dNTPs, an activating enzyme, and a buffer.

Also provided is a method for detecting viruses in a sample, wherein the blood sample contains one or a mixture of viral strains or species, the method comprising the steps of: (a) providing primers targeting viral strains and species; (b) combining primers into a reaction mixture; and (c) performing an amplification reaction with the reaction mixture, wherein the presence of virus is identified by a logic matrix of the amplification products.

Also provided is a method for detecting fungus in a sample, wherein the blood sample contains one or a mixture of viral strains or species, the method comprising the steps of: (a) providing primers targeting fungal strains and species; (b) combining primers into a reaction mixture; and (c) performing an amplification reaction with the reaction mixture, wherein the presence of fungus is identified by a logic matrix of the amplification products.

Also provided is a method for detecting antibiotic resistant bacteria in a sample, wherein the blood sample contains one or a mixture of bacterial strains or species, the method comprising the steps of: (a) providing primers targeting viral strains and species; (b) combining primers into a reaction mixture; and (c) performing an amplification reaction with the reaction mixture, wherein the presence of one or more antibiotic resistant bacteria is identified by a logic matrix of the amplification products. In certain embodiments, the presence of one or more gram positive antibiotic resistant bacteria is detected and identified by the method. Alternatively or in addition, the presence of one or more gram negative antibiotic resistant bacteria is detected and identified.

Also provided is a kit for detecting gram positive and gram negative bacteria, viruses and fungus in a sample, the kit comprising one or more primer targeting gram positive specific bacterial strains and species, gram negative specific bacterial strains and species, viral strains and species and fungal strains and species. In some embodiments, the kit further comprises sample preparation materials for cell lysis for whole blood, or in other embodiments for lysis for separated blood, or in other embodiments for lysis of cells in blood culture. In other embodiments, one or more of the following components are included: dNTPs, an activating enzyme, and a buffer.

Also provided is a kit for detecting viruses and fungus in a sample, the kit comprising one or more primer targeting viral strains and species and fungal strains and species. In some embodiments, the kit further comprises sample preparation materials for cell lysis for whole blood, or in other embodiments for lysis for separated blood, or in other embodiments for lysis of cells in blood culture. In other embodiments, one or more of the following components are included: dNTPs, an activating enzyme, and a buffer.

Exemplary Target Pathogens and Antibiotic Resistance Sequences

Those skilled in the art will appreciate, in light of the present disclosure, that the described target polynucleotides may fall, in some embodiments, into one or more of the following categories: a. a species-specific polynucleotide; a genus-specific polynucleotide; a virulence polynucleotide; an antibiotic resistance polynucleotide; a toxicity polynucleotide; and a generic polynucleotides with at least a region that is conserved between species of pathogen.

The terms “target nucleic acid”, “target polynucleotide”, “target polynucleotide sequence”, “target polynucleotide molecule”, and “target gene” are used interchangeably and synonymously herein and refer to the nucleotide sequence on the template nucleic acid strand to which the primer is intended to hybridize. In various embodiments, the target sequence may comprise an RNA or DNA strand. The terms may refer to a portion of a target gene or to a target gene in its entirety. In another embodiment, a described method or kit utilizes primers for amplifying a target gene or polynucleotide sequence characteristic of (specific for) a species of interest. Those skilled in the art will readily understand that a pair of primers is capable of amplifying a particular target polynucleotide sequence in a PCR reaction if they hybridize to opposite ends of the sequence in an inwardly-pointing direction. In certain embodiments, the gene or polynucleotide sequence may be any gene or polynucleotide sequence whose sequence in the pathogen of interest is unique among common microorganisms. The term “species-specific gene” and “species-specific polynucleotide” are used interchangeably herein to refer to any species-specific sequence or portion thereof, whether a gene or intergenic region.

Antibiotic-resistance polynucleotides of particular interest for the described methods and compositions include the metallo-β-lactamases, including the IMP, vim, and NDM variants (Woodford N, BE SMART Biomerieux Newsletter, October 2012).

It will be appreciated by those skilled in the art that the methods and compositions described herein can be applied to detection of a variety of antibiotic-resistant bacteria, and can utilize a variety of pathogen-specific genes, and antibiotic-resistance genes, for example the following, without intention to limit the scope of the invention to the named pathogens and antibiotic-resistance polynucleotides.

In certain embodiments, as exemplified herein, the target pathogen is S. aureus. In certain other embodiments, the target pathogen is selected from the group consisting of Clostridium Difficile, Staphylococcus aureus, Oerskoviaturbata, Aracanobacterium haemolyticum, Streptococcus bovis, Streptococcus gallolyticus, Streptococcus lutetiensis, Bacillus circulans, Paenibacillus, Rhodococcus, Enterococcus, Klebsiella. In still other embodiments, the target pathogen is selected from the group consisting of bacteria belonging to the Clostridium genus and Eggerthella lenta.

One specific example of a test for association of a mobile genetic element with a particular bacterium is a test for a pathogenic bacterium carrying an antibiotic-resistance gene. Those of skill in the art will understand that genes associated with antibiotic resistance may be located on a cassette or plasmid or may be integrated into a chromosome of the pathogen. In more specific embodiments, the target pathogen is an antibiotic-resistant bacterium.

Reference herein to a polynucleotide that “may be associated with” a particular pathogen covers cases in which the polynucleotide is only carried by a specific pathogen, is carried by a specific family of pathogens, or in non-pathogen specific.

The antibiotic-resistance polynucleotide detected by the described methods and compositions is, in other embodiments, a gene that confers resistance to one or more antibiotic agents selected from the group consisting of methicillin, vancomycin, linezolid, a penicillin-class antibiotic, a cephalosporin-class antibiotic, a carbapenem-class antibiotic, and a monobactam-class antibiotic. In other embodiments, the antibiotic resistance gene is a gene that confers resistance to any other antibiotic agent known in the art.

Bacteria resistant to vancomycin are known in the art, including, in more specific embodiments, vancomycin-resistant Staphylococcus aureus (VRSA) and vancomycin-resistant Enterococcus. Other instances of antibiotic-resistant bacteria are antibiotic-resistant gram-negative bacteria potentially involved in gram-negative bacteria-mediated sepsis. Examples of the latter are cephalosporin-resistant Toxigenic Escherichia coli, as well as E. coli and other gram-negative bacteria, for example Salmonella, Shigella, Campylobacteria, and Yersinia that are resistant to carbapenems (e.g. imipenem and meropenem); penicillins (e.g. piperacillin, ticarcillin and piperacillin/tazobactam); cephalosporins (e.g. ceftazidime and cefepime); monobactams; aminoglycosides; and fluorquinolones.

As is the case for many types of antibiotic-resistance, vancomycin-resistance may be conferred by insertion into the bacterial genome of an element containing a functional van gene, including for example vanA (NCBI Gene ID #9715206), vanB (NCBI Gene ID #'s2598280, 6385877, 4670249, and 4783144), vanB1 (NCBI Gene ID #'s4608418 and 10915848), vanB2 (NCBI Gene ID #'s4607160 and10916198), vanH (NCBI Gene ID #'s7072427 and 2598279), and vanX (NCBI Gene ID #'s7072423, 2598281, and 9988323). The described methods can be used to detect vancomycin resistance polynucleotides through amplification of a region of a van gene by means of appropriate primers. Such primers are designed according to methods known in the art. In one exemplary embodiment, such primers may by complementary to a portion of the van gene. In another exemplary embodiment, such primers may be complementary to polynucleotide sequences outside of the van gene region but that are nevertheless capable of amplifying the van gene region.

Vancomycin resistance has also been detected in strains of Oerskoviaturbata, Aracanobacteriumhaemolyticum, Streptococcus bovis, Streptococcus gallolyticus, Streptococcus lutetiensis, Bacillus circulans, Paenibacillus, Rhodococcus, as well as anaerobic bacteria belonging to the Clostridium genus and Eggerthellalenta. Methods may utilize, in some embodiments, primers capable of amplifying genes and loci that are specific to these strains. In more specific embodiments, a method can be used to detect the presence of vancomycin-resistant (VR) bacteria in a sample and to identify the species and/or strain of the VR bacteria in the sample, using primers directed to at least 2 strain-specific loci, together with primers directed to a van gene region.

The described methods and compositions can, in other embodiments, be used to detect bacteria carrying the New Delhi metallo-β-lactamase gene (NDM-1; NCBI Gene ID: 11933791), including but not limited to the following bacteria: Pseudomonas putida, Pseudomonas pseudoalcaligenes, Escherichia coli, Pseudomonas oryzihabitans, Klebsiellapneumoniae, Shigellaboydii, Sutonellaindologenes, Aeromonascaviae, Stenotrophomonasmaltophilia, Vibrio cholerae, Citrobacterfreundii, Achromobacterspp, Kingelladenitrificans, Pseudomonas aeruginosa, Klebsiellaoxytoca, Enterobacter cloacae, Acinetobacterbaumannii, Proteus mirabilis, Enterobacteraerogenes, Morganellamorganii, and Providenciastuartii.

In certain embodiments, a bridging region polynucleotide sequence may be used as one of the species-specific polynucleotide sequences in the described methods and compositions. The term “bridging region” as used herein refers to a region formed when a mobile genetic element is integrated (i.e. inserted) into the genome of a bacterium. When the term is used in the context of a particular set of primers, it refers to a region capable of being amplified by said set of primers only when the target mobile genetic element is integrated into the genome of the target bacterium. Often, a set of primers used to amplify a bridging region will comprise forward primers that recognize a sequence on the bacterial genome, near the site of integration, and reverse primers that recognize a sequence on the mobile genetic element, or vice-versa. An exemplary, non-limiting example of a bridging region that may be utilized is SCCmec:orfX. Bridging regions are well known in the art, and are described, inter alia, in Cuny and Witte (PCR for the identification of methicillin-resistant Staphylococcus aureus (MRSA) strains using a single primer pair specific for SCCmec elements and the neighboring chromosome-borne orfX. Clin Microbiol Infect. 2005; 11(10):834-7).

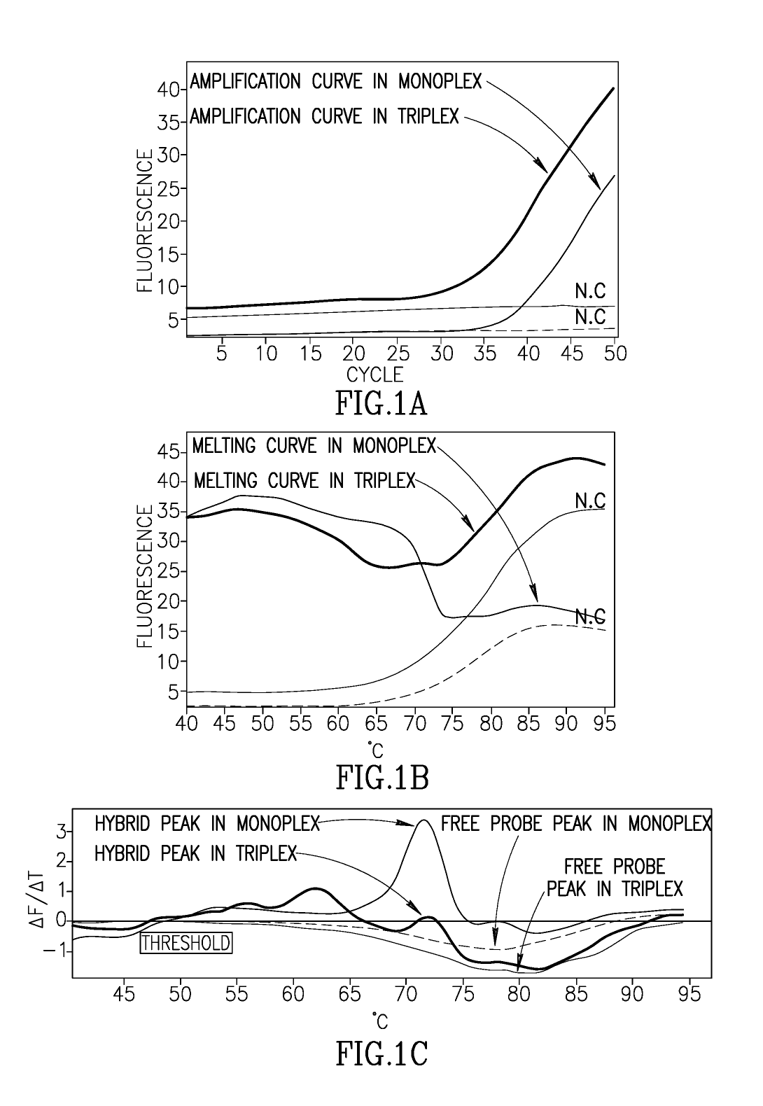

Other examples of bridging regions are comprised of the van sequence region and a region of the bacterial genome that is species and/or strain-specific. For exemplary bridging region polynucleotide sequences, see Launay et al., (2006) Antimicrob. Agents and Chemother. 50(3): 1054-62 and the sequences listed in FIG. 1 of U.S. Pat. No. 8,017,337 as SEQ ID NOs: 25-38 thereof.

Exemplary Sets of Targets

Non-limiting embodiments of the targets of the GP+fungus tube and the GN tube are depicted in Tables 11-12 and Tables 13-14, respectively. Those skilled in the art will appreciate, in light of the present disclosure, that particular targets can be added, eliminated, or moved to the other tube, without adversely affecting the overall efficacy of the assay. For example, the emm target could be removed from the GP+fungus tube. Alternatively or in addition, the oprI target and/or some or all of the IMP primers could be removed from the GN tube.

TABLE 11
Primers of an exemplary, non-limiting GP+ fungus tube.
In this and all primer listings, the ribonucleotide
residue in the sequence is preceded with an “r”.

| | | SEQ |
|---|---|---|
| | | ID |
| Name | Sequence | NO |
| *28S- | GAG TCG AGT TGT TTG GGA ATG CrAG CTC | 46 |
| Aspergillus-F | | |
| *28S- | TTT AAC TCT CTT TTC AAA GTG CTT TTC ATrC | 47 |
| Aspergillus-R | TTT C | |
| *18S fungus- | GGA GTA TGG TCG CAA GGC TrGA AAC | 48 |
| F | | |
| *18S fungus- | AAGAAAGAGCTCTCAATCTGTCArATCCT | 49 |
| R | | |
| L1A1-F | AGAAAAGTTACTAACCCATTAAGAATCCrCTGA | 50 |
| | A | |
| L1A1-R | ATAAGGTGAAGAAACCCCTTTAGArAACTT | 51 |
| 28S-CA-F | CCGGAATGCACGCTCATCAGACrACCAC | 52 |
| 28S-CA-R | GCTACTACCACCAAGATCTGCrACTAG | 53 |
| mecA-F | TGATTATCCATTTTATAATGCTCAAATTTCrAAA | 54 |
| | CA | |
| mecA-R | GCTATAGATTGAAAGGATCTGTACTGGrGTTAA | 55 |
| mecC-F | GATGGGGTACTTACCAAAGCTrAAAAT | 56 |
| mecC-R | TCATTTAACTATAGATGCTAGAGTACAAGAArA | 57 |
| | GTAT | |
| Nuc-F | GGTGATACGGTTAAATTAATGTACAAAGrGTCA | 58 |
| | A | |
| Nuc-R | CTTGCTTCAGGACCATATTTCTCTrACACC | 59 |
| Spa-F | TACATGTCGTTAAACCTGGTGATrACAGT | 60 |
| Spa-R | CCACCAAATACAGTTGTACCGATGrAATGG | 61 |
| IC-F | GCCAGGTCCTCGTTCTCGTrAATCG | 12 |
| IC-R | AGTCAAGTGTGGTTATGGTACTGrUGCGA | 13 |
| 16S-ent-F | AGAGGGGGATAACACTTGGArAACAG | 14 |
| 16S-ent-r | CGTTACCTCACCAACTAGCTAATGrCACCG | 15 |
| Spn9802-F2 | CGA GAT GAT GAA AGC CTT AAG TGT TrAT | 62 |
| | TTT | |
| Spn9802-R | ACC TCT TTC GTA CAT GTA GGA AAC TrAT TTT | 17 |
| Tuf-F | GTGTTGAACGTGGTCAAATCAArAGTTG | 25 |
| Tuf-R | ATTGAACCAGGAGCAGCTAATrACTTG | 26 |
| VanA-F | GGT ATT GGG AAA CAG TGC CGC rGTT AG | 63 |
| VanA-R | CTCGCTCCTCTGCTGAAAGrGTCTG | 64 |
| VanB-F | GATTGTCGGCGAAGTGGATCrAAATC | 65 |
| VanB-R | GCATCCAAGCACCCGATATrACTTT | 66 |
| Emm-F | CTT GAA AAA CTT AAC AAA GAG CTT GrAA | 67 |
| | GAA | |
| Emm-R | CAG CTC TTA GTT TTG CAA GTT CTT CArG CTT | 68 |
| | G | |

*All four fungal markers are not necessary. In practice, 2-3 of the fungal markers may be used.

TABLE 12
Probes of an exemplary, non-limiting GP+ fungus tube. Each
probe is labeled with the indicated fluorophore, where “QS”
stands for Quasar ® and “CFR” stands for Cal Fluor ® Red.
Capital letters signify the hybridization region with the
PCR product. FAM and HEX may be paired with BHQ1; and Cal
Fluor ® Red, QS670, and QS705 paired with BHQ2.

| Probe | | Fluorophore; |
|---|---|---|
| Name | Sequence | SEQ ID NO |
| 28S- | cggccggCTC TAC TTG TGC GCT ATC GGT | FAM/69 |
| Aspergillus- | CTC CGG CCg | |
| PB | | |
| 18S fungus- | cgg GGA CCT GGT GAG TTT CCC CG | FAM/70 |
| PB | | |
| L1A1-PB | cccatcTTT GAT CCA ACT AGA TGG G | FAM/71 |
| 28S-CA-PB | cagggCGGCCGAATGAACTAGCCCTG | FAM/72 |
| mecA-PB | ccaggCACCTTGTCCGTAACCTGg | HEX/73 |
| mecC-PB | ccTGGT TGT AAT GCT GTA CCA Gg | HEX/74 |
| Nuc-PB | cgatgcACA CCT GAA ACA AAG CAT Cg | CFR610/75 |
| Spa-PB | cctggtCAA AGC TCA AGC ATT ACC AGg | CFR610/76 |
| IC-PB4 | ccaGCAAGGGGAAGTGGCTGG | QS670/21 |
| 16S-ent-PB1 | cgGCGA CAC CCG AAA GCG CCg | QS670/22 |
| Spn9802-PB2 | ccttggTTCAAGTCGTTCCAAGG | QS670/24 |
| Tuf-PB3 | caccAGACTACGCTGAAGCTGGTG | CFR610/45 |
| VanA-PB | cgcgagCTGATTTGGTCCACCTCGCg | QS705/77 |
| VanB-PB | cggctATC AGG AAA ACG AGC CG | QS705/78 |
| Emm-PB | ccgaagG CTT TTG CTT CTG CTT Cgg | QS705/79 |

TABLE 13
Primers of an exemplary, non-limiting GN tube. The
multiple primers (and probes) for IMP were developed
to address sequence variability among variants, e.g.
IMP-1, IMP-2, IMP-3, and IMP-4.

| | | SEQ |
|---|---|---|
| | | ID |
| Name | Sequence | NO |
| IMP-F1 | AGA GTC TTT GCC AGA TTT AAA AAT TGA rGAA GC | 80 |
| IMP-F2 | AGT ATT TCC TCT CAT TTT CAT AGC GAC rAGC AC | 81 |
| IMP-F3 | GTT TGT GGA GCG CGG CTA TAA ArAT CAA | 82 |
| IMP-R2 | TTA ACT AGC CAA TAG TTA ACT CCG CTA rAAT GA | 83 |
| IMP-R3 | TAG CTT GTA CCT TAC CGT CTT TTT TrAA GAA | 84 |
| IMP-R4 | CAG TTT TGC CTT ACC ATA TTT GGA CAT TrAA TAA | 85 |
| IMP-R5 | CCC TTT AAC AGC CTG CTC CrCA TGT | 86 |
| OprI-F | TGA ACA ACG TTC TGA AAT TCT CTG CTrC TGG C | 87 |
| OprI-R | CTT GCG GCT GGC TTT TTC rCAG CA | 88 |
| SHV-F | CTG CTG ACC AGC CAG CGT rCTG AG | 89 |
| SHV-R | GCT CTG CTT TGT TAT TCG GGC rCAA GC | 90 |
| CTXM- | GAT GAA CGC TTT CCA ATG TGC AGT rACC AG | 23 |
| 14-F | | |
| CTXM- | TCT GCC AGC GTC ATT GTG CCrG TTG A | 24 |
| 14-R | | |
| CTXM- | GGG CGC AGC TGG TGA CAT GrGA TGA | 25 |
| 15-F | | |
| CTXM- | CGC GAC GGC TTT CTG CCT TArG GTT G | 26 |
| 14-R | | |
| KPC-F | CCA TTC GCT AAA CTC GAA CAG GArC TTT G | 27 |
| KPC-R | AGA AAG CCC TTG AAT GAG CTG rCAC AG | 28 |
| GES-F | CGAC ATT GGT TTT TTT AAA GCC CAG rGAG AG | 29 |
| GES-R | TGA GTT GTG TAA TAA CTT GAC CGA CrAG AGG | 30 |
| OXA-48- | GCG TAG TTG TGC TCT GGA ATG rAGA AT | 31 |
| F | | |
| OXA-48- | GTG TTC ATC CTT AAC CAC GCC CAA rATC GA | 32 |
| R | | |
| 16S-F | CGA AGC AAC GCG AAG AAC CrUT ACC | 5 |
| 16S-R | TTG ACG TCA TCC CCA CCT TrCC TCC | 6 |
| IC-F | GCCAGGTCCTCGTTCTCGTrAATCG | 12 |
| IC-R | AGTCAAGTGTGGTTATGGTACTGrUGCGA | 13 |
| rpoB-F | GGTGGTCAGCGTTTCGGTGAGrATGGA | 33 |
| rpoB-R | TAGTCACCATTTTTTAGTTCAATGTTGrATACC | 34 |
| VIM-F | CAG TCT ACC CGT CCA ATG GTrC TCA T | 1 |
| VIM-R | GAG AAG TGC CGC TGT GTT TTT rCGC AC | 2 |
| NDM-R | TCGACAACGCATTGGCATArAGTCG | 3 |
| NDM-R | AACTGGATCAAGCAGGAGATCrAACCT | 4 |

TABLE 14
Probes of an exemplary, non-limiting GN tube.
See caption to Table 12.

| Probe | | Fluorophore; |
|---|---|---|
| Name | Sequence | SEQ ID NO |
| IMP-PB1 | cccggaAGATTGAGAATTAAGCCACTCTATT | FAM/91 |
| | CCggg | |
| IMP-PB2 | cgccaCA TTT GTT AAT TCA GAT GCA TAC | FAM/92 |
| | GTG Gcg | |
| OprI-PB | cgGGC TAC CGG TTG CAG CAG Cccg | FAM/93 |
| SHV-PB | acctagCGATAAGACCGGAGCTAGgt | HEX/94 |
| CTXM-14- | cggcaTC GAG ATC AAG CCT GCC G | HEX/36 |
| PB | | |
| CTXM-15- | ccccaGACT GCC TGC TTC CTG GGg | HEX/37 |
| PB | | |
| KPC-PB | ccggcTACAGTTGCGCCTGAGCCGG | CFR610/38 |
| GES-PB | ctccgTTCG TCA CGT TCT ACG Gag | CFR610/39 |
| OXA-48-PB | ccgcatGG AAT TTT AAA GGT AGA TGC GG | CFR610/40 |
| 16S-GN-PB | CCGCTcagccatgcagcacctAGCGG | QS705/11 |
| 16S-GP-PB | CGCGCTgacaaccatgcaccacctgAGCGCG | QS670/42 |
| IC-PB | ccaGCAAGGGGAAGTGGCTGG | QS670/21 |
| RpoB-PB | ctcggTTGACCAAAGAGATCCGag | QS670/41 |
| VIM-PB2 | cccgtGCAACTCATCACCATCACGGg | QS705/8 |
| NDM-PB2 | cgcgGCGCGTGAGTCACCACCGCg | QS705/10 |

More examples of antibiotic-resistant pathogens that may be detected by the described methods and compositions are set forth in Table 15 below.

TABLE 15
Non-limiting examples of antibiotic-resistant pathogen strains.

| Examples of Species that | | |
|---|---|---|
| have been affected | Resistance Gene | Antibiotic resistance |
| Pseudomonas aeruginosa | bla_{VIM-2} | Carbapenem-resistance |
| | | from metallo-β- |
| | | lactamase |
| Klebsiella pneumoniae | Bla_{KPC} | Carbapenem-resistance |
| | Klebsiella pneumoniae | from serine-β- |
| | carbapenemase | lactamase |
| Salmonella | gyrAor parC | Ciprofloxacin- |
| | | resistance |
| Many species in the | Bla_{NDM-1} | Carbapenem-resistance |
| Enterobacteriaceae | New Delhi metallo- | from metallo-β- |
| family | beta-lactamase 1 | lactamase |
| Many species in the | bla_{GES} | Carbapenem-resistance |
| Enterobacteriaceae | | from serine-β- |
| family | | lactamase |
| Many species in the | bla_{OXA-48} | Carbapenem-resistance |
| Enterobacteriaceae | | from serine-β- |
| family | | lactamase |
| Many species in the | bla_{IMP} | Carbapenem-resistance |
| Enterobacteriaceae | | from metallo-β- |
| family | | lactamase |
| Many species in the | bla_{TEM} | β-lactamase |
| Enterobacteriaceae | | |
| family | | |
| Many species in the | bla_{SHV} | β-lactamase |
| Enterobacteriaceae | | |
| family | | |
| Many species in the | bla_{LEN} | β-lactamase |
| Enterobacteriaceae | | |
| family | | |
| Many species in the | bla_{CTXM} | β-lactamase |
| Enterobacteriaceae | | |
| family | | |
| Many species in the | bla_{OKP} | β-lactamase |
| Enterobacteriaceae | | |
| family | | |

qPCR and Other Cycle Threshold Amplification Reaction Assays

The described methods and compositions relate to detection and analysis of bacteria and antibiotic-resistance genes using a cycle threshold assay. In general, a cycle threshold assay utilizes a multi-cycle amplification reaction in which the cycle at which a particular amplification product appears relative to other co-amplified fragments can provide information on the strains and species of bacteria present in the sample. In addition, the cycle threshold assay can provide information on the presence of a specific antibiotic-resistant strain, such as, but not limited to, bacterial strains resistant to methicillin. Although the cycle threshold assay is primarily described herein in terms of real-time PCR, it will be appreciated that other template-based amplification reactions, including isothermal amplification reactions (non-limiting examples of which are helicase-mediated amplification reactions and Loop-mediated isothermal amplification [LAMP]), can be adapted for use using methods known in the art and described further herein. Other types of amplification reactions that may be utilized include, for example, nucleic acid sequence-based amplification (NASBA), self-sustained sequence replication (3SR), strand displacement amplification (SDA) and branched DNA signal amplification (bDNA).

In some embodiments, PCR is used as the amplification method in the described assays. PCR is an in vitro technique for the enzymatic synthesis of specific DNA sequences using 2 oligonucleotide primers that hybridize to complementary nucleic acid strands and flank a region that is to be amplified in a target DNA. A series of reaction steps, including (1) template denaturation, (2) primer annealing, and (3) extension of annealed primers by DNA polymerase, results in the exponential accumulation of a specific fragment whose termini are defined by the 5′ ends of the primers. The term “PCR” as used herein encompasses derivative forms of the reaction, including but not limited to real-time PCR, quantitative PCR, multiplexed PCR, reverse transcription PCR and the like.

“Real-time PCR” refers to a PCR method in which the amount of reaction product, i.e. amplicon, is monitored as the reaction proceeds. There are many forms of real-time PCR, which differ mainly in the detection chemistries used for monitoring the reaction product, e.g. Gelfand et al, U.S. Pat. No. 5,210,015 (“TaqMan®”); Wittwer et al, U.S. Pat. Nos. 6,174,670 and 6,569,627 (intercalating dyes, such as SYBER® Green); Tyagi et al, U.S. Pat. No. 5,925,517 (molecular beacons); all of which are incorporated herein by reference in their entirety for all purposes and in particular for their teachings regarding real-time PCR. Other exemplary detection chemistries include, but are not limited to Scorpion Primers, Sunrise Primers, and Eclipse Probes. Detection chemistries for real-time PCR are reviewed in Mackay et al, (2002) Nucleic Acids Research, 30:1292-1305, which is also incorporated herein by reference in its entirety for all purposes, and in particular for its disclosure of different detection chemistries for real-time PCR.

The described methods are intended for use with any type of PCR reaction, either quantitative (“real-time”) or non-quantitative. “Quantitative PCR” or “qPCR” refers to a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: (β-actin, GAPDH, (β2 microglobulin, ribosomal RNA, and the like. Techniques for quantitative PCR are well-known to those of ordinary skill in the art, as exemplified in the following references: Freeman et al, (1999) Biotechniques, 26: 112-126; Becker-Andre et al, (1989) Nucleic Acids Research, 17: 9437-9447; Zimmerman et al., (1996) Biotechniques, 21: 268-279; Diviacco et al, (1992) Gene, 122: 3013-3020; U.S. Pat. No. 6,664,080 to Klaus Pfeffer, entitled “TaqMan™-PCR for the detection of pathogenic E. coli strains”; Paitan (ibid); U.S. Pat. No. 6,329,138 to Hans De Beenhouwer et al, entitled “Method for detection of the antibiotic resistance spectrum of mycobacterium species”; U.S. Pat. No. 7,045,291 to Nancy Hanson et al, entitled “Multiplex PCR for the detection of AmpC beta-lactamase genes”; U.S. Pat. Nos. 5,994,066 and 6,001,564 to Bergeron et al, assigned to Creighton University; and International patent application WO/1996/008582 and US Pat. App. No. 2004/0185478, each to Bergeron et al. Each of these patents and applications is incorporated herein by reference.

Primers

Generally amplification methods used in the described methods will utilize primers as starting points for the amplification of the template in each cycle of the reaction. In such reactions, primers anneal to a complementary site on the template (also referred to herein as “target”) polynucleotide, and then enzymes such as DNA polymerase are used to extend the primers along the sequence of the template polynucleotide. As will be appreciated, the assays described herein may utilize mixtures of primers that include primers comprising only naturally occurring DNA and/or RNA nucleotides, primers containing non-naturally occurring nucleotides, primers containing modifications such as those described herein and known in the art, primers containing a combination of modifications and non-naturally and naturally occurring nucleotides, and any combination thereof.

Primers typically have a length in the range of from about 5 to about 50, about 10 to about 40, about 12 to about 30, and about 20 to about 25 nucleotides. The length of the primers is typically selected such that the primers bind at the desired annealing temperature with optimal selectivity to a target polynucleotide sequence(s).

Generally, primers are used as pairs which include a “forward” primer and a “reverse” primer, with the amplification target of interest lying between the regions of the template polynucleotide that are complementary to those primers. The design and selection of appropriate PCR primer sets is a process that is well known to a person skilled in the art. Automated methods for selection of specific pairs of primers are also well known in the art, see e.g. U.S. Publication No. 2003/0068625. In one embodiment, a set of amplification primers can be selected such that the distance between the two primers on the amplicon is at least 5 base pairs (bp). In other embodiments, the primers are selected such that the distance is about 5 to about 50, about 10 to about 40, and about 20 to about 30 bp. In one embodiment, amplicons resulting from real-time PCR methods are from about 50 to about 400 bp, from about 75 to about 300, from about 100 to about 200 and from about 180 to about 400 bp. In certain embodiments, the amplicon does not exceed 200 bp. Primers described as “for”, “directed to”, or “capable of amplifying” a particular target sequence are complementary to the ends of the target sequence, with the 3′ ends facing inward, such that the target sequence can be amplified in a PCR reaction.

In some embodiments, primers used are modified to reduce non-specific hybridization, such as those described in U.S. Pat. Nos. 6,001,611; 6,482,590; 6,794,142; and US Pat. App. Nos. 2007/0128621; 2007/0281308; 2003/0119150; 2003/0162199; 2009/0325169; 2010/0167353; and International Pat App. Nos. WO 2009/004630; PCT/IB2010/054613, each of which is hereby incorporated by reference in its entirety for all purposes and in particular for all teachings related to modified primers.

The terms “DNA base”, “RNA base”, “nucleotide”, “nucleoside”, “nucleotide residue”, and “nucleoside residue” as used herein refer to deoxyribonucleotide or ribonucleotide residues, or other similar nucleoside analogues capable of serving as components of primers suitable for use in a PCR reaction. Such nucleoside and derivatives thereof are used as the building blocks of the primers described herein, except where indicated otherwise. Nothing in this application is mean to preclude the utilization of nucleoside derivatives or bases that have been chemical modified, for example to enhance their stability or usefulness in a PCR reaction, provided that the chemical modification does not interfere with their recognition by DNA polymerase as deoxyguanine, deoxycytosine, deoxythymidine, or deoxyadenine, as appropriate.

In certain embodiments, some or all of the primers used in a PCR amplification performed in conjunction with the described methods and compositions are riboprimers. Riboprimers are described inter alia in US Pat. App. Nos. 2009/0325169 and 2010/0167353, both assigned to Integrated DNA Technologies Inc. (IDT) and entitled “RNase H-Based Assays Utilizing Modified RNA Monomers”, and in US Pat. App. No. 2011/0086354, entitled “Methods and Compositions for Multiplex PCR Amplifications”, to Tzubery, Tzvi et al.

In certain embodiments, primers used include an inactivating chemical modification that is reversed by the action of an activating enzyme present in the amplification mixture. As will be appreciated, the assays described herein may utilize mixtures of primers that include primers comprising only naturally occurring nucleotides, primers containing non-naturally occurring nucleotides, primers containing modifications such as those described herein and known in the art, primers containing a combination of modifications and non-naturally and naturally occurring nucleotides, and any combination thereof.

Probes

The referred-to primers may or, in other embodiments, may not be detectably labeled. In one aspect, the products of amplification reactions are detected using labeled primers. In another aspect, such products are detected using probes directed to particular regions of the template nucleic acid. In still another aspect, the assay is a molecular-beacon based assay. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., (1998) Nature Biotechnology. 16:49).

The term “probe” as used herein refers to an oligonucleotide, either natural or synthetic, that is generally detectably labeled and used to identify complementary nucleic acid sequences by hybridization. Primers and probes may have identical or different sequences. “Probe suitable for real-time PCR” refers to any probe that emits a detectable signal in real-time in the presence of the target sequence, including those described in U.S. Pat. Nos. 5,925,517, 6,037,130, 6,103,476, 6,150,097, 6,461,817 and 7,385,043, which are incorporated herein by reference.

In another embodiment, the probe is a dual-modified oligonucleotide, as utilized in the Examples herein. Dual-modified oligonucleotides are well known in the art, and are described, inter alia, in International patent application WO 2008/063194 and in US App. Pub. Nos. 2009/0068643, 2009/0325169, and 2010/0167353. These include, but are not limited to, TaqMan® Probes, Eclipse™ and Molecular Beacons. An exemplary, non-limiting type of suitable probe is a Molecular Beacon. Use of Molecular Beacons is well known in the art, and is described, inter alia, in Tyagi S and Kramer F R (1996) Molecular beacons: probes that fluoresce upon hybridization. Nat Biotechnol 14, 303-308. Molecular Beacons and other probes suitable for real-time PCR typically include a fluorescent reporter molecule at the 5′-end and a quencher molecule at the 3′-end. Probes modified with any one of an extensive group of fluorophores are commercially available and referenced in the Product Catalogs of suppliers such as Integrated DNA Technologies, Inc. (Coralville, Iowa), Eurogentec North America Inc. (San Diego, Calif.) and Biosearch Technologies Inc. (Novato, Calif.). Non-limiting examples include FAM, HEX, TET, ROX, Texas Red, Cy 5, TYE 665, TYE 563, Quasar carboxylic acids and Quasar active esters. As is well known to those skilled in the art, the selection of an appropriate quencher moiety is determined by the fluorescence emission of the probe's fluorophore and, includes, but is not limited to Black Hole Quencher-1, Black Hole Quencher-2, Black Hole Quencher-3, Iowa Black FQ, Iowa Black RQ-Sp, Dabcyl, Deep Dark Quencher I, Deep Dark Quencher II and Deep Dark Quencher III.

Alternatively or in addition, Taqman probes are used for the controlled melt (instead of molecular beacon), together with the Taq polymerase that has been modified to not digest the Taqman probes upon target labeling, such as a Taq polymerase that lacks a 5-3 nuclease activity, as described, for example, in Luo et al.

In still other embodiments, Taqman probes are used, in some embodiments in combination with Taq polymerase, as is known in the art and is described, inter alia, in Holland, PM et al (1991), “Detection of specific polymerase chain reaction product by utilizing the 5′-3′ exonuclease activity of Thermus aquaticus DNA polymerase”. PNAS USA 88(16): 7276-7280.

In other embodiments, the probes are any other type of probes known in the art. Those of skill in the art will understand in light of the disclosure provided herein that a variety of types of probes may be utilized in the described amplification reactions without appreciably affecting performance, and that any combination of different fluorophores and quenchers can be readily used for each of the probes in the reaction mixture. Each possibility may be considered as being a separate embodiment.

Methods for Detecting Target Sequences of Interest in a Test Sample

In some embodiments, the described methods and compositions amplify nucleic acids from the test sample. It will be understood by those skilled in the art that test samples containing intact cells will be typically subject to a lysis procedure prior to performing the PCR reaction. In certain embodiments, the sample lysate has not been subjected to a nucleic acid purification procedure prior to the amplification reaction. In other embodiments, the sample lysate may have been subjected to a crude nucleic acid purification procedure, but not an extensive one. In certain embodiments, the described methods overcome difficulties encountered with amplification of non-purified nucleic acid samples. Methods of preparing pathogen DNA from blood samples are known in the art with 50% yield and are commercially available, for example the MolYsis Basic10 kit from Molzym, Bremen, Germany.

Test Samples

The term “test sample” as used herein refers to any nucleotide-containing sample suspected of containing a target sequence, for instance a sample suspected of containing a pathogen of interest or human or animal DNA marker of interest. In certain embodiments, the test sample is a clinical specimen from a mammal. In certain other embodiments, the test sample is a clinical specimen from a human. In more specific embodiments, the test sample may be a blood sample from a human. In other embodiments, the test sample is a DNA extract of a blood sample from a human, or in other embodiments, a bacterial DNA extract of a blood sample from a human. The term “clinical specimen” as used herein refers alternatively to a specimen obtained from processing a body fluid, tissue, or any type of biopsy from a mammal.

In certain embodiments, the clinical specimen is a body fluid. In another embodiment, the clinical specimen is nasal fluid. In another embodiment, the clinical specimen is a nasal swab. In another embodiment, the clinical specimen is a swab from an armpit. In another embodiment, the clinical specimen is a swab from a groin, in certain embodiments a vaginal swab or a perineal swab. In another embodiment, the clinical specimen is whole blood. In another embodiment, the clinical specimen is serum. In another embodiment, the clinical specimen is plasma. In another embodiment, the clinical specimen is cerebrospinal fluid. In another embodiment, the clinical specimen is urine. In another embodiment, the clinical specimen is lymph fluid. In another embodiment, the clinical specimen is tears. In another embodiment, the clinical specimen is saliva. In another embodiment, the clinical specimen is milk of a subject. In another embodiment, the clinical specimen is amniotic fluid. In another embodiment, the clinical specimen is an external secretion of the respiratory tract. In another embodiment, the clinical specimen is an external secretion of the intestinal tract. In another embodiment, the clinical specimen is an external secretion of the genitourinary tract. In another embodiment, the clinical specimen is selected from the group consisting of nasal fluid, vaginal secretions, whole blood, serum, plasma, cerebrospinal fluid, urine, lymph fluids, tears, saliva, milk, amniotic fluid and an external secretion of the respiratory, intestinal or genitourinary tract of a subject in need of testing for a target sequence of interest. Each possibility may be considered as being a separate embodiment.

In another embodiment, the clinical specimen is a tissue from a biopsy of a subject. Typically, the tissue will have been treated appropriately (for example, by homogenization), to render it a substrate for PCR amplification. In certain embodiments, the tissue is white blood cells. In certain embodiments, the tissue is a malignant tissue. In certain embodiments, the tissue is chorionic villi. In certain embodiments, the tissue is selected from the group consisting of white blood cells, malignant tissues, and chorionic villi. In certain embodiments, the tissue comprises a cell type selected from the group consisting of white blood cells, malignant tissues, and chorionic villi. In certain embodiments, the tissue consists essentially of a cell type selected from the group consisting of white blood cells, malignant tissues, and chorionic villi. Each possibility may be considered as being a separate embodiment. In other embodiments, one of the following sample types is used to diagnose the corresponding disorder:

| Application | Sample Type |
|---|---|
| Arthritis | Synovial Fluids |
| Endocarditis | Heart Valve |
| Implant Infection | Smear from Prosthesis |
| Meningitis | Cerebrospinal Fluid (CSF) |
| Periodontitis | Smear from Deep Neck |
| Peritonitis | Ascites Fluid |
| Pleuritis | Pleural Fluid |
| Pneumonia | Bronchoalveolar Lavage |
| Sample from Blood Culture | Blood Culture Identification |
| Sepsis, Neutropenic Fever | Blood |
| Tick borne Disease | Blood, CSF, Ascites Fluids |
| Wound Infection, Biopsy | Pus, Abscess, Smear, Tissue |

It will be appreciated by those skilled in the art that samples for use in clinical, environment, sanitary, or veterinary applications can be used in accordance with the methods, compositions, and kits described herein.

Kits

Provided, in another embodiment, is a kit comprising a described PCR reaction mixture and instructions for use thereof, for example for amplifying specific target sequences in clinical specimens. In another embodiment, the kit is indicated for detecting a pathogen in a test sample and contains instructions for the detection.

In other embodiments, the described kits comprise reaction mixes for use in real-time amplification assays. Such reaction mixes can be stabilized mixtures containing all the constituents for performing the reaction in one or more containers (such as tubes for use in a PCR machine). In an exemplary embodiment, such stabilized reaction mixtures include primers and fluorescently-labeled probes. In a further embodiment, such mixtures are stabilized such that they can be stored at room temperature.

Other aspects provide a kit for of detecting the presence of a gram-positive bacterium in a test sample and the presence of a polynucleotide sequence associated with antibiotic resistance in a GP bacterium, the kit comprising: (a) a described GP reaction mixture; (b) a DNA polymerase enzyme; and (c) deoxynucleoside triphosphates (dNTPs). In certain preferred embodiments, the kit also comprises magnesium. In some embodiments, the magnesium ions are provided separately from the other components. Alternatively or in addition, the primer sets of the kit are asymmetric, and the probes are designed to hybridize to the excess product in a sequence-specific fashion. Each embodiment of the reaction mixtures described herein—for example a GP mixture, a GN mixture, a fungal mixture, a GP+fungal mixture, a GN+fungal mixture, and a GP+GN mixture—and their components should be considered a separate embodiment in the context of this kit.

Other aspects provide a kit for of detecting the presence of a gram-positive bacterium and/or a fungus and/or the presence of a polynucleotide sequence associated with antibiotic resistance in a GP bacterium, the kit comprising: (a) a described GP+fungal reaction mixture; (b) a DNA polymerase enzyme; (c) deoxynucleoside triphosphates (dNTPs); and (d) magnesium. In certain preferred embodiments, the kit also comprises a probe suitable for real-time PCR. Alternatively or in addition, the primer sets of the kit are asymmetric, and the probes are designed to hybridize to the excess product in a sequence-specific fashion. Each embodiment of the reaction mixtures described herein and their components should be considered a separate embodiment in the context of this kit.

Other aspects provide a kit of detecting the presence of a gram-negative bacterium in a test sample and the presence of a polynucleotide sequence associated with antibiotic resistance in a GN bacterium, the kit comprising: (a) a described GN reaction mixture; (b) a DNA polymerase enzyme; (c) deoxynucleoside triphosphates (dNTPs); and (d) magnesium. In certain preferred embodiments, the kit also comprises a probe suitable for real-time PCR. Alternatively or in addition, the primer sets of the kit are asymmetric, and the probes are designed to hybridize to the excess product in a sequence-specific fashion. Each embodiment of the reaction mixtures described herein and their components should be considered a separate embodiment in the context of this kit.

Other aspects provide a kit for confirming and determining the cause of a suspected case of sepsis, the kit comprising: (a) a described GP reaction mixture, or in other embodiments a described GP+fungal reaction mixture; (b) a described GN reaction mixture; (c) a DNA polymerase enzyme; (d) deoxynucleoside triphosphates (dNTPs); and (e) magnesium. In certain preferred embodiments, the kit also comprises a probe suitable for real-time PCR. Alternatively or in addition, the primer sets of the kit are asymmetric, and the probes are designed to hybridize to the excess product in a sequence-specific fashion. Each embodiment of the reaction mixtures described herein and their components should be considered a separate embodiment in the context of this kit.

Those of skill in the art will appreciate, in light of the present disclosure, that in some embodiments, the described kit will contain both (a) PCR reagents, and (b) software capable of directing a computer to analyze the fluorescence data in accordance with one or more logic matrices derivable from this disclosure. In certain embodiments, the program is physically present in the kit box on digital media such as a CD. In other embodiments, the program is provided as part of the kit in the form of an instruction in the kit User Manual that directs the user of the kit to download a software program from a specified location such as the kit supplier's website. In still other embodiments, the program is provided as part of the kit in the form of a kit User Manual instruction that directs to the user of the kit to use a particular software program provided on a data storage device such as a USB drive.

In other embodiments, the aforementioned software is loaded onto a computer. Typically, this computer also interfaces with the fluorescence reader and inputs data from same. In some embodiments, the computer belongs to the end user, while in other embodiments, the computer or processor is provided as part of the kit. In preferred embodiments, the software directs the computer to (a) access a file containing data from the fluorescence reader and (b) analyze these data.

Thus, those of skill in the art will appreciate, in light of the present disclosure, that the PCR reagents and software provided as part of the described kit, together with other reagents and equipment which may be provided either as part of the kit or by the user, for example water, test tubes, a thermocycler, and a computer or computer system, will enable a laboratory worker or technician or an automated sample processor to carry out a described method. The PCR reagents and software, operating with said other reagents and equipment, perform, in some embodiments, analysis of a sample suspected of containing a target pathogen, possibly carrying an antibiotic-resistance gene.

In still other embodiments, the described kit further comprises a set of primers for amplifying at least a portion of an additional polynucleotide sequence characteristic of said bacterium. In more specific embodiments, the additional polynucleotide sequence is associated with integration of the target mobile genetic element into the target bacterium.

In other embodiments, the divalent cation used in the described methods and compositions is stored and/or provided separately from the other components of the reaction mixture, and may be withheld until after the template is added. In other embodiments, the divalent cation is provided together with the other components of the reaction mixture.

Hydration-Reduced PCR Reaction Mixtures

In light of the disclosure provided herein, those skilled in the art will appreciate that the described compositions and methods are compatible with both ordinary and storage-stabilized PCR reaction mixtures, such as but not limited to hydration-reduced PCR reaction mixtures. The reaction mixtures utilized in the Examples herein were treated to reduce hydration and were stored at room temperature until use. However, very similar, if not identical, results may be obtained with ordinary PCR reaction mixtures. Thus, in one embodiment, the described PCR reaction mixtures are hydration-reduced PCR reaction mixtures. In another embodiment, they are ordinary reaction mixtures. In another embodiment, they are any type of reaction mixtures known in the art. Each possibility may be considered as being a separate embodiment.

Hydration-reduced PCR reaction mixtures that are ambient temperature-stabilized are further described in co-pending US patent application 2008/0050737. Such mixtures are prepared by hydration-reducing solutions containing DNA polymerase and/or dNTPs, and also containing a buffer compound containing at least one stabilizing agent, and are stored at a temperature between 25° C.-100° C., typically about 55° C. The stabilizing agent(s) may be inter alia a sugar and a protein, for example sucrose and/or BSA. Typically, 1-20% sucrose and 0.5-3 mg/ml BSA are included. In another embodiment, any other type of hydration-reduced PCR mixture known in the art is utilized. In another embodiment, any other type of ambient temperature-stabilized PCR mixture known in the art is utilized. Each possibility may be considered as a separate embodiment.

In other embodiments, the reaction mixture is lyophilized to increase its storage life.

Reference is now made to the following Examples, which, together with the above descriptions, illustrate certain embodiments of the invention in a non-limiting fashion. The Examples are representative of a large amount of research that was performed to iteratively improve many aspects of the multiplex reactions described herein.

### EXPERIMENTAL DETAILS SECTION

### Introduction

The studies described herein were performed in order to improve the ability of PCR assays of DNA extracts of blood samples to distinguish between the presence of various agents capable of causing sepsis, as well as the presence of various common antibiotic-resistance genes. The goal was to provide actionable results, i.e. a recommended antibiotic regimen, within a few hours, as opposed to several days for culturing, the gold-standard diagnostic method.

Sepsis patients can have as few as 1 pathogen DNA copy per milliliter (ml) of blood. Since only 10 ml of blood is typically drawn, and at least 50% of the pathogen DNA can be lost during purification, it is important to split the sample into as few tubes as possible. These considerations led to the development of a 2-tube assay, with each tube yielding 12-15 answers, where each “answer” refers to the presence or absence of a particular nucleotide sequence. The 2 tubes are referred to throughout the Example section as the “Gram-Positive” (or “GP”) and “Gram-Negative” (or “GN”) tubes, as shown below in Tables 1-2. (These titles may not align exactly with every primer in the tubes. For example, this embodiment of the GP tube contains fungal targets, and this embodiment of the GN tube contains a general marker for GP bacteria).

TABLE 1
Target amplicons of an exemplary, non-limiting embodiment
of the GP tube.

| | Exemplary SEQ | |
|---|---|---|
| | ID NOs: forward | |
| Target | primer(s)/reverse | |
| Name | primer(s)/probe(s) | Comments |
| 28S | 46/47/69 | See notes to Tables 11-12 regarding |
| Aspergillus | | the fungal markers. |
| 18S fungus | 48/49/70 | General fungal marker |
| L1A1 | 50/51/71 | Marker for Candida albicans. |
| (Candida | | |
| albicans) | | |
| 28S Candida | 52/53/72 | General marker for candida & |
| & | | aspergillus. |
| Aspergillus | | |
| mecA | 54/55/73 | Methicillin resistance. |
| mecC | 56/57/74 | Variant of mecA. |
| nuc | 58/59/75 | Staphylococcus aureus marker |
| spa | 60/61/76 | Additional S. aureus marker |
| IC | 12/13/18-21 | Modified jellyfish DNA sequence |
| | | included as positive control for |
| | | amplification and fluorescence |
| | | detection. |
| 16S | 14/15/22 | Specific for 16S of E. faecium and |
| | | E. faecalis |
| Spn9802 | 16/17/23-24 | S. pneumoniae marker |
| tuf | 25/26/43-45 | Generic Staphylococcus marker |
| vanA | 63/64/77 | Vancomycin resistance |
| vanB | 65/66/78 | Vancomycin resistance |
| emm | 67/68/79 | Group A, C, and G beta-hemolytic |
| | | streptococcus |

TABLE 2
Target amplicons of an exemplary, non-limiting embodiment of the
GN tube.

| | Exemplary SEQ ID NOs: | |
|---|---|---|
| Target | forward primer, reverse | |
| Name | primer, probe. | Comments |
| IMP | 80-82/83-86/91-92 | |
| OprI | 87/88/93 | |
| SHV | 89/90/94 | |
| CTXM-14 | 23/24/36 | |
| CTXM-15 | 25/26/37 | |
| KPC | 27/28/38 | |
| GES | 29/30/39 | |
| OXA-48 | 31/32/40 | |
| 16S | 5/6/11, 42 | Probes 11 and 42 detect GN and |
| | | GP 16S rRNA, respectively. |
| | | Primers 5 & 6 amplify both targets. |
| IC | 12/13/18-21 | Same as IC for other tube. |
| rpoB | 33/34/41 | |
| vim | 1/2/7-8 | |
| NDM | 3/4/9-10 | |

### Materials and Experimental Methods—General

qPCR Assays

qPCR was performed using asymmetric amplification, with ribo-primers (US Pat. App. Nos. 2009/0325169 and 2010/0167353), with the excess and limiting primer in each set present at 1 μM and 0.1 μM concentrations, respectively.

Experimental Setup

Amplification and detection reactions were run using RotorGene™ 6000 RotorGene™ Q PCR instruments (Qiagen). The following standard qPCR protocol was used:
1. 3 minutes at 95° C. to denature the DNA.
2. 50 amplification cycles, each consisting of the following three steps: (a) 15 seconds (sec) at 95° C.; (b) 50 sec at 56° C.; (c) 20 sec at 72° C. (at the end of step (b), the readings were taken for each of the five fluorescent dyes).

The amplification was followed by a controlled melt, as follows: 60 sec at 95° C., 90 sec at 40° C., then heating to 95° C. at a rate of 1° C. each 5 sec. Readings were taken for the 5 channels at the end of the 5 sec incubation at each temperature.

Controlled Melt

The sample was heated for 60 sec 95 deg, then the temperature was dropped to 40 deg and held at 40 deg for 90 sec. The sample was then heated to 95 in 1 degree increments, stopping for 5 sec and measuring fluorescence at each step.

Internal Control Polynucleotide

The internal control polynucleotide was a modified Jellyfish DNA, purchased from Integrated DNA Technologies, Coralville, Iowa. This double-stranded DNA contains 2 complementary strands, each blocked by chemical modification at their 3′ ends, which were hybridized prior to commencing the assay.

qPCR Reaction Mix

The PCR reaction mixture contained DNA Polymerase (Taq Pol from Jena Bioscience, Germany), dNTP's (Jena Bioscience), Tris-HCl, pH 8.3, KCl, BSA, and BSA to stabilize Taq polymerase.

Fluorophores

The following fluorophores are used throughout the document, unless indicated otherwise: FAM (“green”), HEX (“yellow”), Cal Fluor® Red 610 (“orange”), Quasar® 670 (“red”), and Quasar®705 (“crimson”).

### EXAMPLE 1

### Proof of Concept of Successful Identification of Bacteria and Resistance Genes in Clinical Samples

### Materials and Experimental Methods

Primers

The primers and probes used in the initial study are show in Tables 17-18, respectively:

TABLE 17
Primers used in initial study. The letter
“r” indicates a ribonucleotide residue
in the following residue

| SEQ | | |
|---|---|---|
| NO. | Name | Sequence |
| 103 | 16S-Ent-F2 | AGAGGGGGATAACACTTGGArAACAG |
| 104 | 16S-Ent-R2 | CGTTACCTCACCAACTAGCTAATGrCACCG |
| 105 | MecA-F2 | TAGCACTCGAATTAGGCAGTAAGrAAATT |
| 106 | MecA-R2 | GCTATAGATTGAAAGGATCTGTACTGGrGTTAA |
| 107 | MecC-F2 | GATGGGGTACTTACCAAAGCTrAAAAT |
| 108 | MecC-R2 | CACATTATTGGAGAAAAAGGCTGAArAACGG |
| 109 | Nuc-F2 | GGTGATACGGTTAAATTAATGTACAAAGrGTCAA |
| 110 | Nuc-R2 | CTTGCTTCAGGACCATATTTCTCTrACACC |
| 111 | Spa-F2 | TACATGTCGTTAAACCTGGTGATrACAGT |
| 112 | Spa-R2 | CCACCAAATACAGTTGTACCGATGrAATGG |
| 113 | Tuf-F2 | GTGTTGAACGTGGTCAAATCAArAGTTG |
| 114 | Tuf-R2 | ATTGAACCAGGAGCAGCTAATrACTTG |
| 115 | Eae-F | AGAACGGTAATAAGAAGTCCAGTGrAACTA |
| 116 | Eae-R | GCCAGGCTTCGTCACAGTrUGCAG |
| 117 | vanA-F2 | GTTGTGCGGTATTGGGAAACrAGTGC |
| 118 | vanA-R2 | CTCGCTCCTCTGCTGAAAGrGTCTG |
| 119 | vanB-F2 | GATTGTCGGCGAAGTGGATCrAAATC |
| 120 | vanB-R2 | GCATCCAAGCACCCGATATrACTTT |

TABLE 18
Probes used in initial study. Each probe was labeled with the
indicated fluorophore. Capital letters signify the
hybridization region with the PCR product.

| SEQ | | | Fluorophore & |
|---|---|---|---|
| ID | Name | Sequence | Quencher |
| 121 | 16S-Ent- | CGCGATCcatcagcgacacccgaaagcgccttGA | FAM/BHQ1 |
| | PB2 | TCGCG | |
| 122 | mecA-PB2 | CCATGCGagctgattcaggttacggacaaggtgaaa | HEX/BHQ1 |
| | | CGCATGG | |
| 123 | MecC-PB2 | CCATGCGggttgtaatgctgtaccagatccatcgtcat | HEX/BHQ1 |
| | | tCGCATGG | |
| 124 | Nuc-PB2 | CGCGATCttggttgatacacctgaaacaaagcatcct | Cal Fluor ®/ |
| | | GATCGCG | BHQ2 |
| 125 | Spa-PB2 | CGCGATCgaacttgttgttgataagaagcaaccagca | Cal Fluor ®/ |
| | | GATCGCG | BHQ2 |
| 126 | Tuf-PB4 | CGCCAGTccgtaaattattagactacgctgaagctggt | Quasar ® 670/ |
| | | gaACTGGCG | BHQ2 |
| 127 | Eae-PB | CGCCAGTctctgcagattaacctctgccgttccataat | Quasar ® 670/ |
| | | gtACTGGCG | BHQ2 |
| 128 | vanA-PB2 | CGCTGACgaggtggaccaaatcaggctgcagtacg | Quasar ® 705/ |
| | | gaaGTCAGCG | BHQ2 |
| 129 | vanB-PB2 | CGCTGACtcttccgcatccatcaggaaaacgagccg | Quasar ® 705/ |
| | | GTCAGCG | BHQ2 |

PCR

The PCR began with a 3 min denaturation at 95 deg, followed by 43 cycles as follows:
5 sec 95 deg,
50 sec 56 deg. (fluorescence readings in 5 channels),
20 sec 72 deg.

Clinical Performance Evaluation

The clinical evaluation was performed using residual patient samples of a 700-bed hospital. At the Medical Center, blood samples are analyzed for micro-organisms and their antibiotic resistance upon amplification using the BACTEC™ system (Becton Dickinson, BD) and a combination of molecular, biochemical and microbiology methods.

The Gram-Positive Sepsis Panel was tested in a two-part clinical study of 171 samples. The first part comprising 51 clinical samples was an open study. The second part, a double blind study comprised 120 clinical samples. Blood drawn from patients was incubated in the BD BACTECT™ blood culture system, a fully automated microbiology growth and detection system designed to detect microbial growth from blood samples.

Blood samples utilizing three types of blood culture bottles were included in the study:
Plus AerobicT™/F
Plus Anaerobic™/F Medium and
Peds PlusT™/F Medium

Blood samples were incubated for up to 6 days, with positive samples identified by the BACTEC system after an average of 17 hours of incubation. A negative sample was defined as a sample which did not reach the threshold established in the BACTEC system in 6 days. Samples reaching the threshold and thus identified as positives were then subjected to the hospital's standard clinical diagnostic protocol, which included Gram staining, selection microbiology platting, PCR and biochemical analysis, including bioMérieux's Vitek® 2 automated biochemical analysis system. All the samples used in the study were analyzed and a diagnosis reached by the hospital team. The samples for the study were numbered by the Director of the Hospital's Microbiology Laboratory and all patient identification and characterization information was removed. The investigator was requested to select samples based on high complexity and not according to prevalence of microbial species.

### Results

Preliminary experiments were performed to show that qPCR amplification can be used to differentially diagnose VRE bacteria containing vanA or vanB genes, Enterococcus not containing vanA or vanB genes (“Entero”), MRSA, MSSA, MRCNS, MSCNS, Vancomycin-resistance & MRSA (“VR+MRSA”), Vancomycin-resistance & MSSA (“VR+MSSA”), Vancomycin-resistance & MRCNS (“VR+MRCNS”), Vancomycin-resistance & MSCNS (“VR+MSCNS”) and various mixed bacterial samples.

Open study clinical evaluation: The first part of the kit evaluation, an open clinical evaluation was conducted using 51 characterized samples. The bacterial diagnosis of each sample was identified by the hospital, and the residual blood culture bottle transferred to the laboratory, together with the hospital diagnosis. 10 μl of blood culture solution from each bottle was processed using the reagents and protocol of the Gram-Positive Sepsis Panel. The hospital's clinical diagnosis was then compared to the results from the Gram-Positive Sepsis Panel. Samples with discrepant results were subjected to further testing using a standard microbiology protocol designed to analyze and identify discrepancies.

All 51 clinical samples from the first part of the study were correctly identified by the Gram-Positive Sepsis Panel, with 44 matching the hospital's diagnosis. Seven discrepant samples were subjected to discrepancy analysis with the Panel diagnosis confirmed by subsequent microbiology testing. Discrepant results were also communicated to the investigator, who performed additional testing and confirmed the new results.

Double-Blind clinical evaluation: The second part clinical evaluation was conducted using an additional 120 samples, in a blinded clinical study design. The numbered residual clinical blood culture bottles selected for the study were transferred to the laboratory for processing. The study was performed using the reagents and protocol of the Gram-Positive Sepsis Panel, by laboratory staff blind to the sample's identity and diagnosis. A file containing the results from the Gram-Positive Sepsis Panel was transferred to an individual in the laboratory, followed by transfer from the hospital of a file containing the hospital's diagnosis for each sample. The hospital's clinical diagnosis was then compared to the results from the Gram-Positive Sepsis Panel. Samples, with discrepant results were subjected to further testing using the standard microbiology protocol.

Out of 120 samples, the Gram-Positive Sepsis Panel identified 116 matching the hospital's diagnosis. From the 4 non-matching samples, 2 were correctly identified by the Gram-Positive Panel, and the correct diagnosis confirmed by subsequent standard microbiology testing. Of the two remaining discrepant samples, both were identified as “false positive” using the study criteria, one of them was later shown by more extensive microbiology analysis to be a true positive finding. More specifically, one of the two “false positive” findings was identified by the hospital's diagnosis as containing Enterococcus and identified by Gram-Positive Sepsis Panel as containing a mixture of Enterococcus and MRSA. A follow-up series of dilutions of the original blood culture was prepared and plated on several selective agar plates over 7 days. The follow-up analysis results confirmed the Gram-Positive Sepsis Panel finding, disclosing a high concentration of Enterococcus and a small, but clearly present, additional quantity of MRSA bacteria.

Strains included in the clinical study were as follows:
25—MRSA
25—MSSA
14—Entero
31—MRCNS
17—MSCNS
1—Mixed Sample: Entero+MRCNS
37—Negative for the bacteria of the kit, but positive for other bacteria or fungi
21—Negative for any bacteria or fungus

Total results from too two-part clinical study

Reference

| | | + | − | Total |
|---|---|---|---|---|
| Gram-Positive Sepsis Panel | + | 113 | 2 | 115 |
| | − | 0 | 56 | 56 |
| | Total | 113 | 58 | 171 |
| Sensitivity | 100% | | | |
| Specificity | 97% | | | |
| PPV | 98% | | | |
| NPV | 100% | | | |

Analytical Inclusivity

Analytic inclusivity of the Gram-Positive Sepsis Panel was demonstrated using a collection of 32 characterized strains reflecting a range of genetic diversity relevant to the kit. The samples included:
6—MRSA
4—MSSA
7—Vancomycin sensitive Enterococcus (VSE)
4—MRCNS
4—MSCNS
4—Vancomycin resistant Enterococcus (vanA positive)
3—Vancomycin resistant Enterococcus (vanB positive)

The Analytical Inclusivity study included strains that were not present in the above clinical study, including Not Vancomycin resistance (Van A or B). For testing, each bacterial sample was spiked into a mixture of residual clinical blood culture sample identified as negative for the presence of any bacteria or fungi. All of the samples were tested in duplicate, at below the clinically relevant level of detection for Gram-positive blood culture sample, with all but one tested at a concentration equal to ˜440 colony forming units (CFU) per reaction tube. Each of the samples was correctly identified by the Gram-Positive Sepsis Panel.

Analytical Specificity—Cross Reactivity

Samples containing purified DNA from five non-target organisms that may be present in human blood were tested. The samples included two distinct E. coli strains, one Enterobacter cloacae, one Streptococcus pyogenes and one Candida. Each sample was tested in duplicate, at a concentration equal to ˜250,000 CFU per reaction tube. Each of the samples was correctly identified as negative by the Gram-Positive Sepsis Panel.

Analytical Specificity—Microbial Interference

One MRSA strain, one MRCNS strain and one Enterococcus strain were each tested at low concentrations (˜220 CFU per reaction tube) in mixtures with high concentrations of DNA from non-target organisms (˜250,000 CFU per reaction tube). The non-target organisms included 3 bacteria (2 E. coli and 1 S. pyogenes) and one type of yeast (Candida). Each sample was tested in duplicate and all were correctly identified by the Gram-Positive Sepsis Panel.

### EXAMPLE 2

### Modification of the Vim Probe

In order to address the challenge of highly-multiplexed PCR in a single tube, multiple probes were designed that had varying affinities for the respective target polynucleotides. We performed 4-channel real-time PCR, followed by a high-resolution melting (HRM) assay in the presence of SYBR® Green in an attempt to use the combination of amplification readouts and melting signatures to distinguish the presence of 15 different targets. However, it proved very challenging to distinguish this number of signatures from each other with sufficient resolution to produce unambiguous results.

Next, it was decided to adopt a different approach, namely multi-channel PCR, namely with five different channels, with multiple probes in each channel, and using asymmetric primer sets, such that linear-after-exponential amplification occurred, resulting in an excess of one strand of the product. Multiple probes were designed that had varying affinities for the respective single-stranded (ss) PCR products, in order that the probes in each channel would have distinguishable hybrid melting signatures. The first step was to optimize the channels individually. This and the next few Examples (through Example 5) describe the optimization of the crimson channel of the “Gram-Negative” (“GN”) assay tube.

Probes and Primers (Examples 2-5)

Crimson Forward and Reverse Primers of the Gram-Negative Assay Tube

The forward and reverse primers of the “crimson” targets (i.e. targets amplified by crimson probes) in the GN assay tube, depicted in Table 3, amplified a fragment of the vim gene (SEQ ID NOs: 1-2) and NDM (SEQ ID NOs: 3-4), both metallo-β-lactamases involved in carbapenem resistance, and the gene encoding the 16S ribosomal RNA (rRNA) of GN bacteria (hereinafter “16S-GN”) (SEQ ID NO: 5-6). The primers were designed to amplify a wide range of known variants of these two genes, namely variants 1-7 in the case of NDM, and vim variants 1, 2, 3, 4, 5, 6, 8, 9, 10, 11, 12, 14, 15, 16, 17, 18, 19, 20, 23, 24, 25, 26, 27, 28, 30, 31, and 32.

In each case, either the forward or reverse primer was present in 10-fold excess, resulting in asymmetric (linear after exponential) amplification of a single strand after the limiting primer was consumed.

TABLE 3
GN forward and reverse primers for targets of
crimson probes. The letter “r” indicates a
ribonucleotide residue in the following
residue. The 16SGPN primers amplify the gene
encoding the 16S subunit rRNA of both GP and
GN bacteria.

| SEQ | | |
|---|---|---|
| NO. | Name | Sequence |
| 1 | VIM-F | CAG TCT ACC CGT CCA ATG GTrC TCA T |
| 2 | VIM-R | GAG AAG TGC CGC TGT GTT TTT rCGC |
| | | AC |
| 3 | NDM-F | TCGACAACGCATTGGCATArAGTCG |
| 4 | NDM-R | AACTGGATCAAGCAGGAGATCrAACCT |
| 5 | 16SGPN-F | CGA AGC AAC GCG AAG AAC CrUT ACC |
| 6 | 16SGPN-R | TTG ACG TCA TCC CCA CCT TrCC TCC |

Crimson Probes of the “Gram-Negative” Assay Tube

Dual-labeled Molecular BeaconT™ probes were used, as depicted in Table 4, recognizing the excess strand of the vim amplicon (SEQ ID NOs: 7-8), the NDM amplicon (SEQ ID NOs: 9-10), or the 16S-GNamplicon (SEQ ID NO: 11). Each of these probes was labeled with Quasar® 705 and BHQ2 on its 5′ and 3′ ends, respectively. Capital letters signify the hybridization region with the PCR product.

TABLE 4
GN Crimson Probes.

| SEQ | | | | |
|---|---|---|---|---|
| ID | Name | Sequence | Length | ΔAM |
| 7 | VIM- | cgccgtgCAATCAAAAGCAACTCATCACCA | 38 | 6.3 |
| | PB1 | TCACGGcg | | |
| 8 | VIM- | cccgtGCAACTCATCACCATCACGGg | 26 | 6.6 |
| | PB2 | | | |
| 9 | NDM- | cgccgGTCCTGATGCGCGTGAGTCACCAC | 34 | 3.9 |
| | PB1 | CGgcg | | |
| 10 | NDM- | cgcgGCGCGTGAGTCACCACCGCg | 24 | 9.9 |
| | PB2 | | | |
| 11 | 16SGN- | ccgctCAGCCATGCAGCACCTagcgg | 26 | 16 |
| | PB | | | |

Results

Samples containing the vim gene were amplified and detected with the VIM-1 probe. This probe worked well alone; however, it worked unexpectedly poorly in a triplex reaction where the other 2 crimson probes, NDM-PB1 and 16S-GN, were present (FIG. 1). The relatively low signal-to-noise ratio in the triplex reaction is best visualized in FIG. 1C, as evidenced by the relatively low height of the hybrid peak compared to magnitude of the downward trend at slightly higher temperatures. This was determined to be due to a strong free probe background from the other probes.

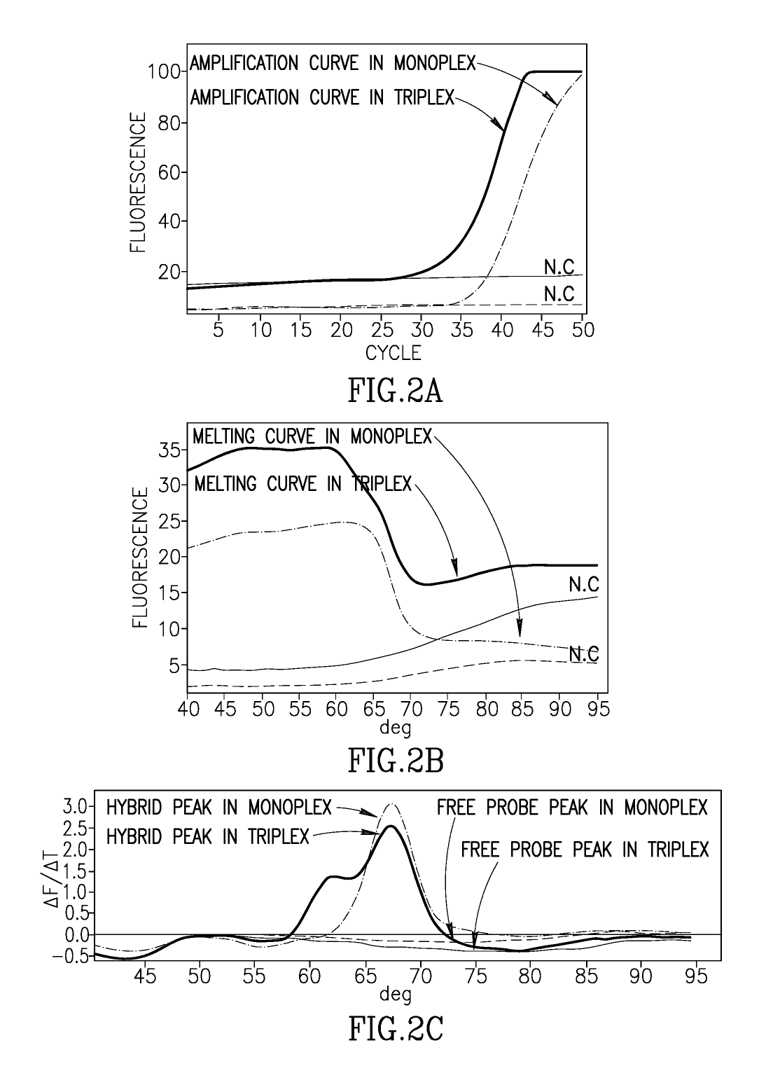

A new probe, VIM-PB2, was designed, by reducing the probe length. The probe was designed in shared-stem format in order to enable the shortening without reducing the melting temperature (“T_{M}”) of the hybrid of the probe to the desired target. As used throughout the Examples, “ΔT_{M}” refers to the difference between the internal melting temperature (“internal T_{M}”) of the probe and the T_{M} of the probe-target hybrid, as measured empirically, where a positive value indicates that the internal T_{M} is higher. Without wishing to be bound by theory, reducing the length of the probe is believed to reduce the background from fluorescence of the free probe that has undergone internal melting (i.e. opening of the stem-loop structure). Preliminary research showed that, for the majority of the primers described herein, a ΔTM of 7-13 was ideal. In some cases, where 3 discriminable melting signatures per channel were desired, a ΔTM of about 7-10 was preferable. Additionally, the NDM probe was improved and renamed NDM-PB2, as described in the following Example. Another triplex amplification was performed, this time using VIM-PB2, NDM-PB2, and 16S-GN-PB. The vim probe performed significantly better in this reaction (FIG. 2).

### EXAMPLE 3

### Modification of the NDM Probe

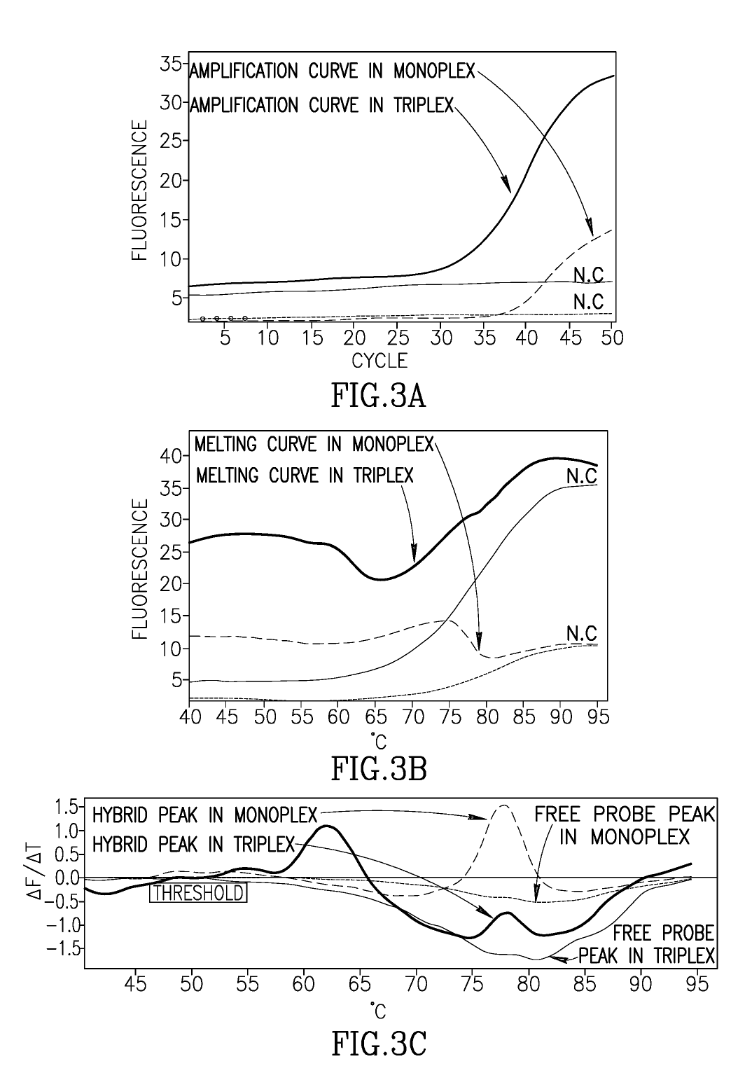

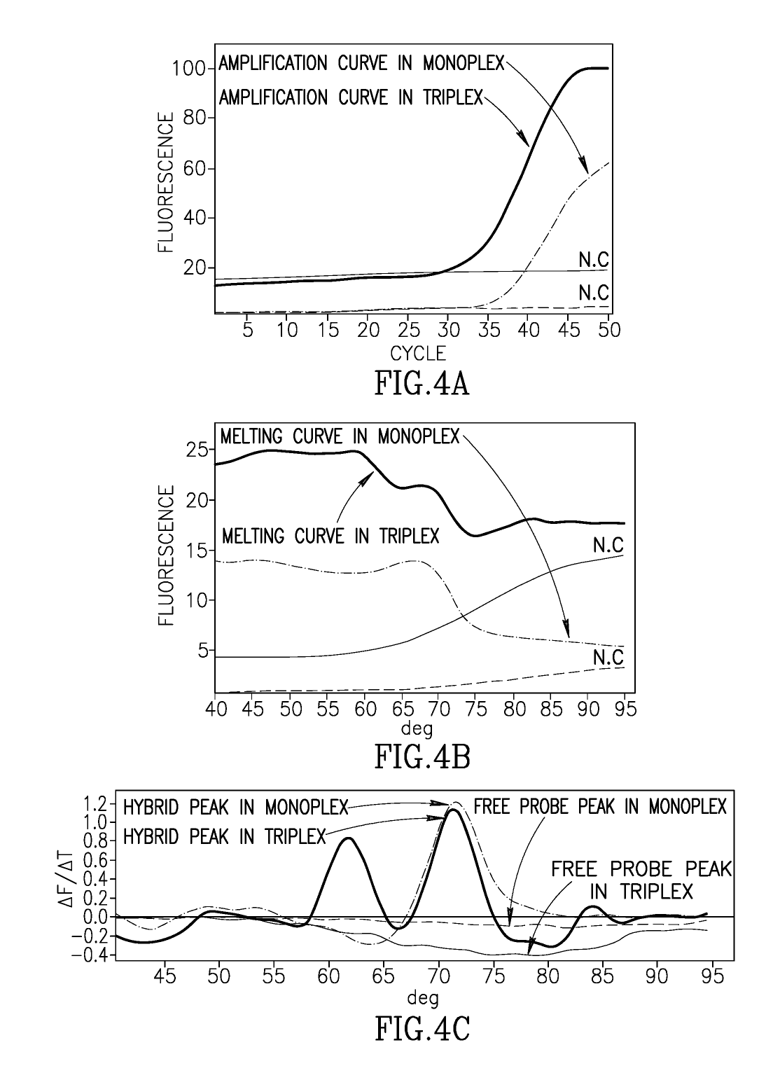

Similarly to the vim probe, the initial NDM probe, NDM-PB 1, worked well in alone, but unexpectedly exhibited a reduced signal-to-noise ratio when the other crimson probes, VIM-PB1 and 16S-GN-PB, were present (FIG. 3). This was determined to be due to a strong free probe background from the other probes. The probe was improved by reducing its length while simultaneously increasing its ΔTM to fall within the desired range, resulting in NDM-PB2. A triplex amplification was performed with VIM-PB2, NDM-PB2, and 16S-GN-PB. Similar to the vim probe, the NDM probe exhibited a significantly improved signal-to-noise ratio in this reaction (FIG. 4).

### EXAMPLE 4

### Performance of the 16S-GN Probe With Initial and Improved Versions of the Vim and NDM Probes

The 16SGN-PB probe was designed to specifically detect the gene for the GN 16S subunit rRNA. This was done by exploiting a sequence variation between the GN and GP 16S RNA, and designing the probe such that it would have a higher affinity for the gene for GN 16S RNA. Thus, in the context of the described assay, the presence of GN vs. GP 16S RNA can be determined based on the melting signature of the 2 different probes, each of which recognizes the relevant single-strand PCR product, but does not sufficiently bind the other 16S PCR product to produce a signal.

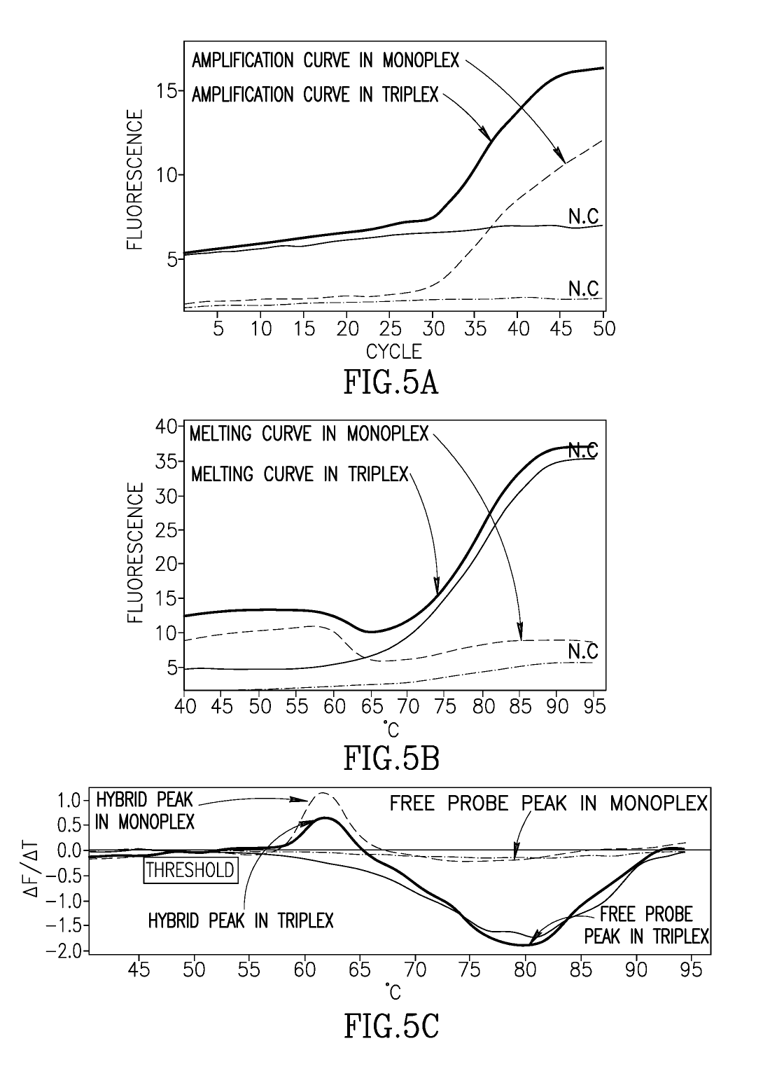

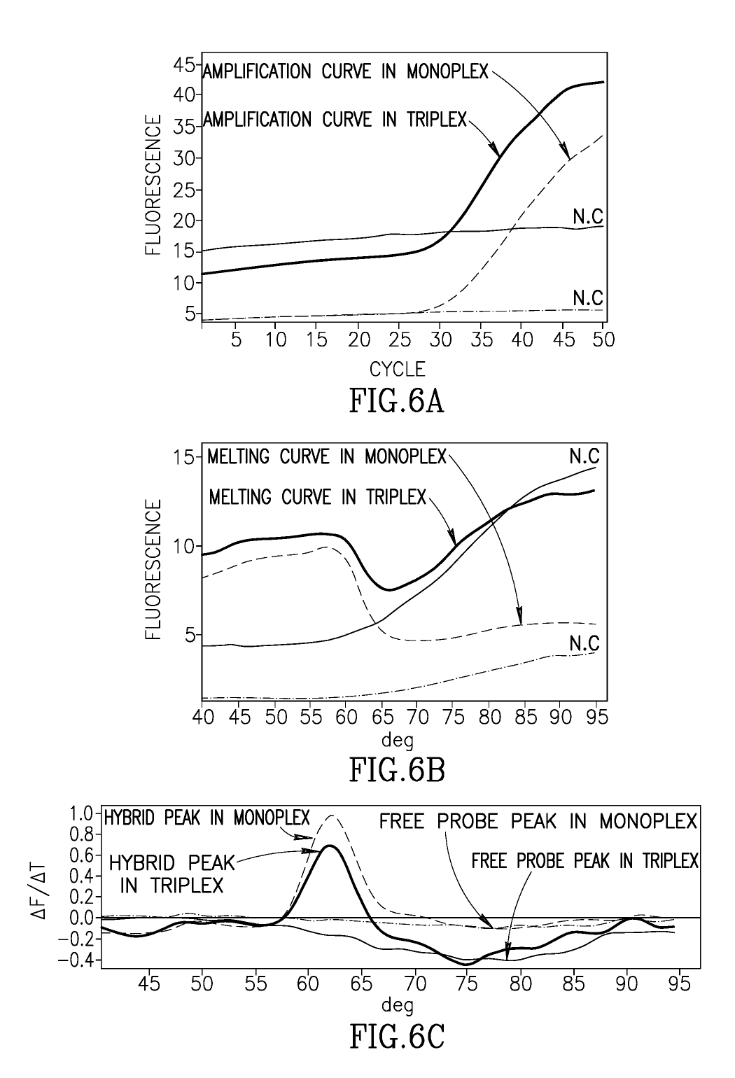

16SGN-PB worked well in the absence of other probes. In the presence of VIM-PB1 and NDM-PB1, it produced a clear melting peak, but the signal-to-noise ratio was less than optimal (FIG. 5). This is shown best in FIG. 5C, where the magnitude of the peak, corresponding to a decrease in fluorescence resulting from melting of the probe from the hybrid, is relatively small compared to the trough, produced by an increase in fluorescence due to the internal melting of free probe. This problem was also addressed by the aforementioned improvements in the vim and NDM probes. The triplex in the presence of VIM-PB2 and NDM-PB2 produced a much-improved signal-to-noise ratio (FIG. 6).

### EXAMPLE 5

### Overall Improvement of Crimson Channel Probes of Gram-Negative Tube When Used Together

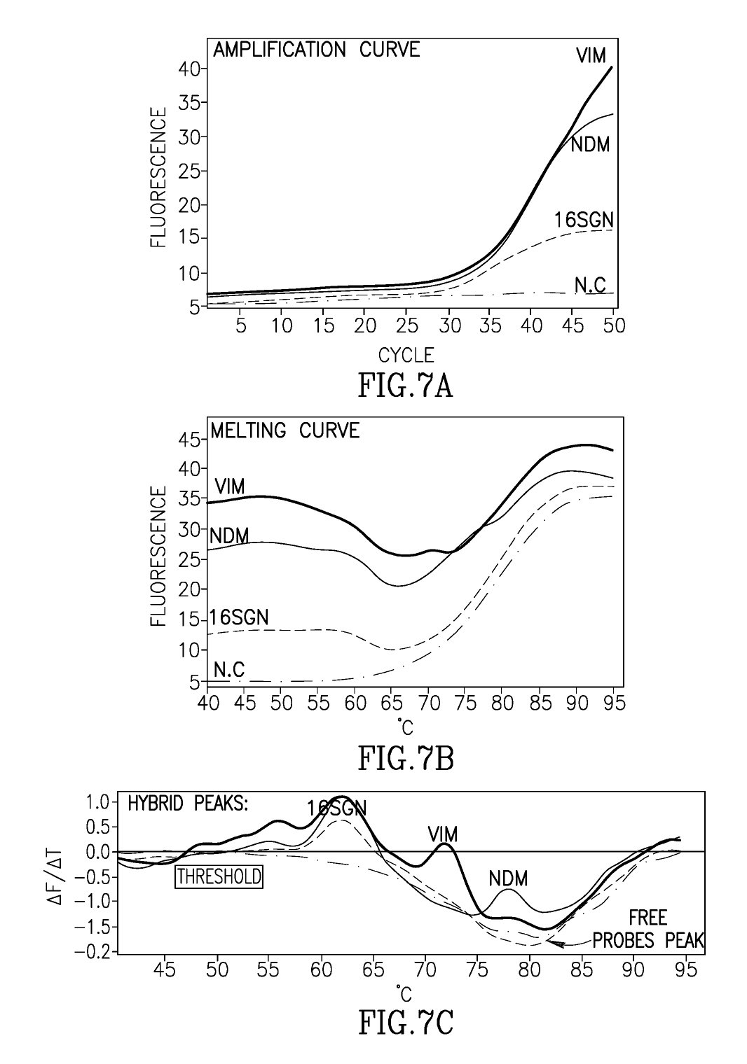

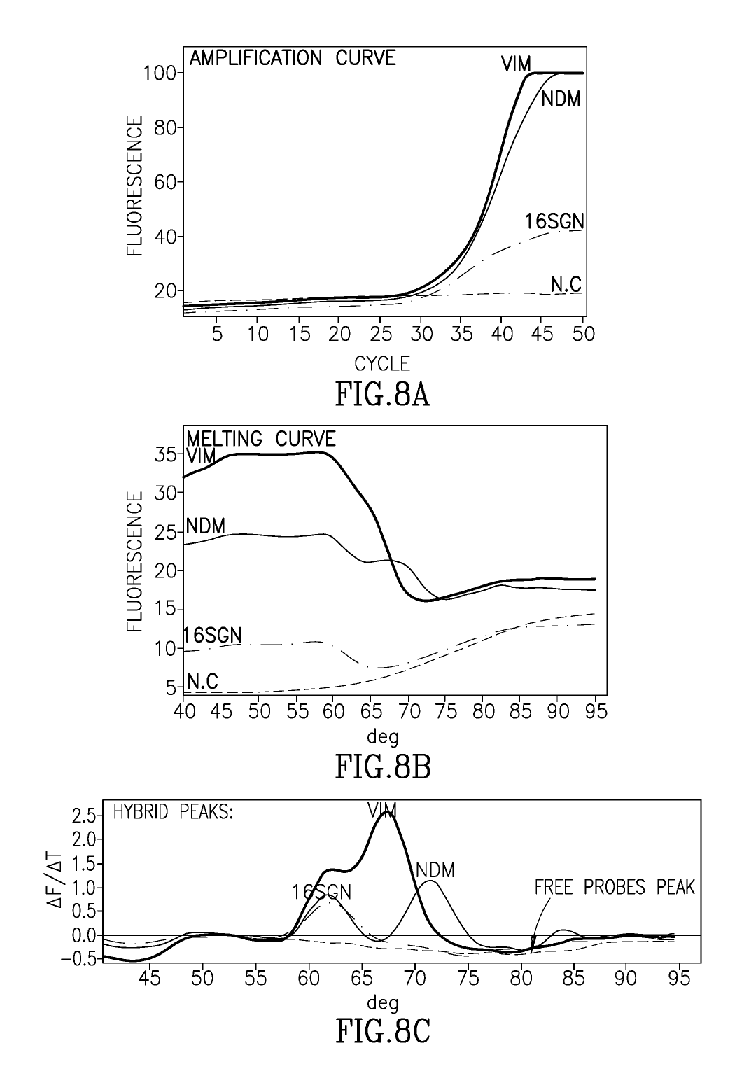

Amplifications of 3 separate tubes containing vim+16SGN, NDM+16SGN, or 16SGN were performed, in each case in the presence of VIM-PB1, NDM-PB1, and 16SGN-PB. Superimposition of the curves from these reactions showed that VIM-PB1 and NDM-PB1 exhibited a poor signal-to-noise ratio of fluorescence (FIG. 7; see specifically FIG. 7A). By contrast, when the experiment was repeated with VIM-PB2, NDM-PB2, and 16SGN-PB, all probes exhibited an acceptable signal-to-noise ratio (FIG. 8). All tubes showed a signal for 16SGN, which may have been due to the slight 16SGN contamination in the preparations of the template vim and NDM plasmids.

### EXAMPLE 6

### Modification of the Spn9802 Probe

Probes and Primers (Examples 6-8)

Red Forward and Reverse Primers of the Gram-Positive Assay Tube

The forward and reverse primers of the red targets of the GP assay tube, depicted in Table 5, amplified the IC (SEQ ID NOs: 12-13); E. faecium and E. faecalis 16S (SEQ ID NOs: 14-15), and Spn9802 (SEQ ID NO: 16-17). The primers and probes for the 16S subunit rRNA gene are included in these tables for completeness, even though optimization of this probe is not described herein. The 16S probe exploited a sequence variation that enabled specific detection of E. faecium and E. faecalis 16SrRNA as opposed to rRNA of other species.

TABLE 5
GP forward and reverse primers for targets
of red probes. The ribonucleotide residue
in the sequence is preceded with an “r”.

| SEQ | | |
|---|---|---|
| NO. | Name | Sequence |
| 12 | IC-F | GCCAGGTCCTCGTTCTCGTrAATCG |
| 13 | IC-R | AGTCAAGTGTGGTTATGGTACTGrUGCGA |
| 14 | Ent16S-F | AGAGGGGGATAACACTTGGArAACAG |
| 15 | Entl6S-R | CGTTACCTCACCAACTAGCTAATGrCACCG |
| 16 | Spn9802- | GGT AAC AAG TCT AGA TCA GAT TGA |
| | F1 | AGC rUGA TA |
| 17 | Spn9802- | ACC TCT TTC GTA CAT GTA GGA AAC |
| | R | TrAT TTT |

Red Probes of the “Gram-Positive” Assay Tube

The Red GP probes are depicted in Table 6, recognizing the excess strand of the IC (SEQ ID NOs: 18-21), the E. faecium and E. faecalis 16S amplicon (SEQ ID NO: 22), or the Spn9802amplicon (SEQ ID NOs: 23-24). Each of these probes was labeled with Quasar® 670 and BHQ2 on its 5′ and 3′ ends, respectively. Capital letters signify the hybridization region with the PCR product.

TABLE 6
GN Crimson Probes.

| SEQ | | | | |
|---|---|---|---|---|
| ID | Name | Sequence | Length | ΔTM |
| 18 | IC-PB1 | ccggGGACCTGCTCTTCCAGCCACTTCC | 32 | 0 |
| | | CCgg | | |
| 19 | IC-PB2 | cgcgtCAGGTCCTGCACGCG | 20 | 17.5 |
| 20 | IC-PB3 | cgccACGTGCAAGGGGAAGTGGCg | 24 | 18 |
| 21 | IC-PB4 | ccaGCAAGGGGAAGTGGCTGG | 21 | 7 |
| 22 | Ent16S- | cgGCGA CAC CCG AAA GCG | 21 | 11.5 |
| | PB1 | CCg | | |
| 23 | Spn9802- | CgctcACGATACAAAGAAAATATTCAAG | 34 | 12.5 |
| | PB1 | TGAGCg | | |
| 24 | Spn9802- | ccttggTTCAAGTCGTTCCAAGG | 23 | 7.5 |
| | PB2 | | | |

Results

This and the next two Examples describe individual modification of two of the red probes of the GP tube, which amplified Spn9802 and the internal control (“IC”), which was a modified jellyfish DNA sequence.

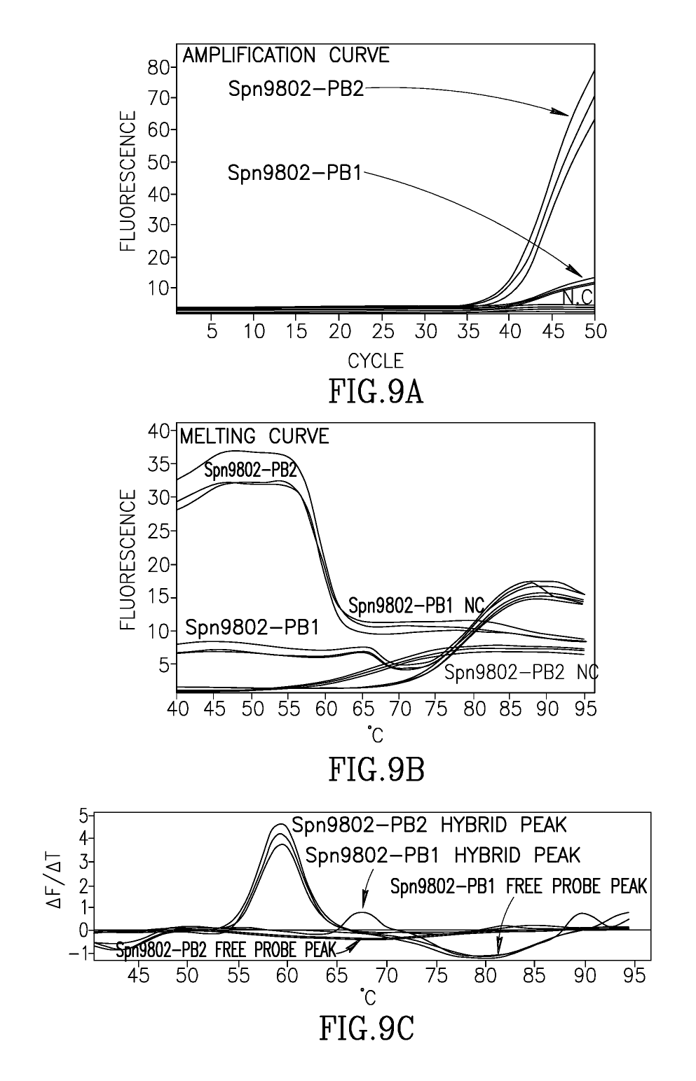

The Spn9802 probe was designed to recognize the Streptococcus pneumoniae chromosomal fragment known as “Spn9802”. This fragment correlates with clinical disease mediated by S. pneumoniae (Abdeldaim et al 2008). The initial probe, Spn9802-PB1, did not exhibit a sharp melting peak. This was believed to be partially due to the high background from free probe. Additionally, the large ΔTM was believed to excessively favor the stem-loop structure over the hybrid. Therefore, the probe was shortened while reducing its ΔTM. The resulting probe, Spn9802-PB2, performed significantly better (FIG. 9).

### EXAMPLE 7

### Modification of the IC Probe

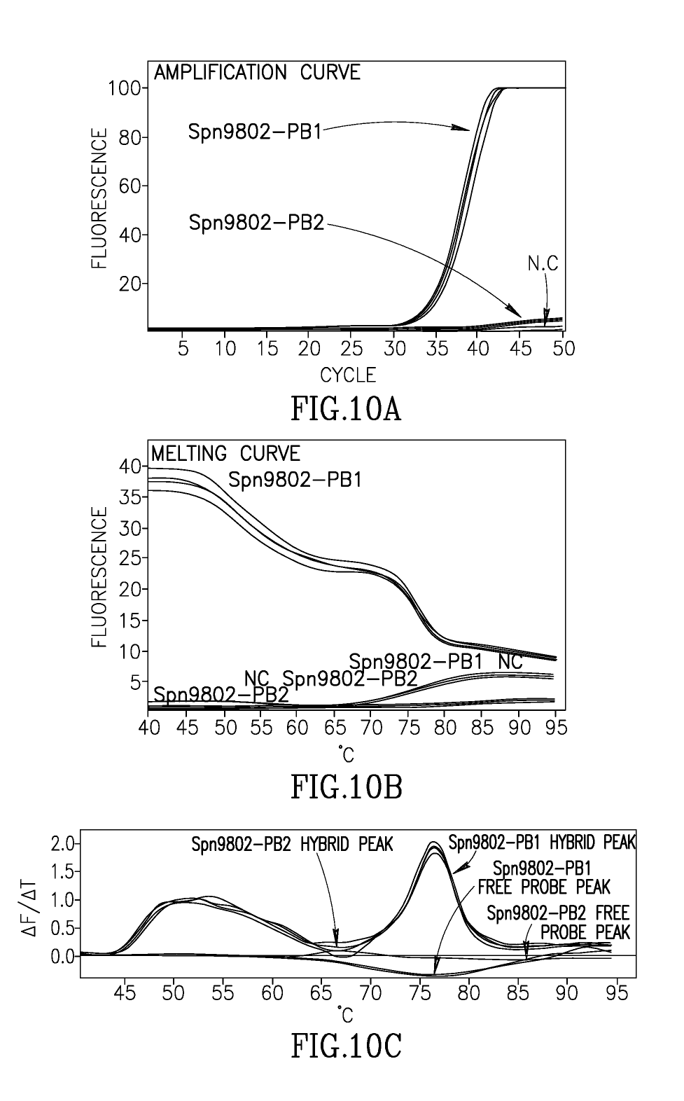

The initial IC probe, IC-PB 1, produced an easily detectable hybrid peak, yet had a high free probe background and thus was unsuitable for triplex. The probe was shortened to reduce the free probe background. The resulting probe, IC-PB2, had a lower free probe background, but also a much lower hybrid peak (FIG. 10).

### EXAMPLE 8

### Further Modification of the IC Probe

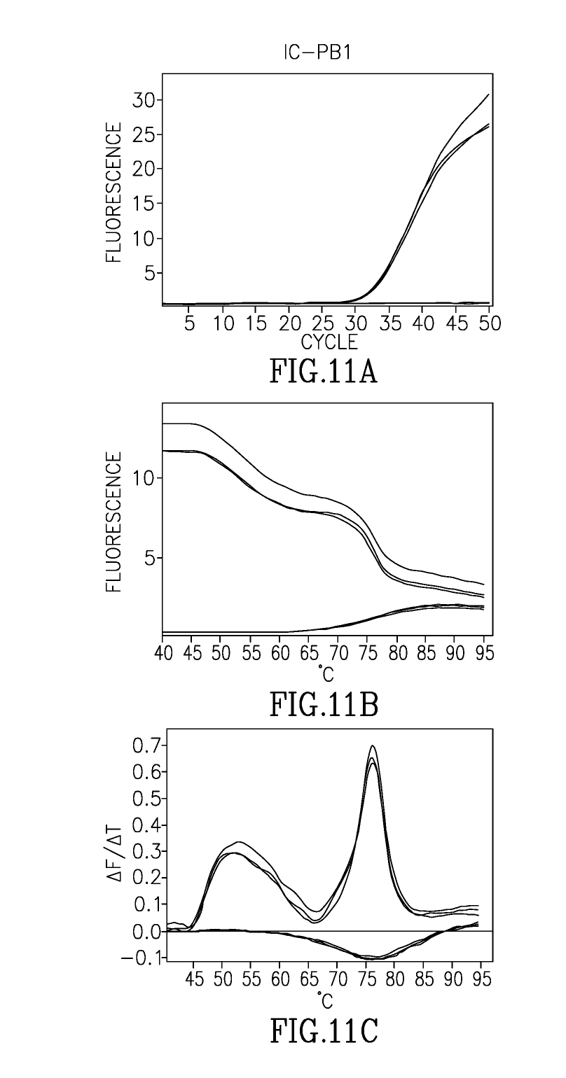

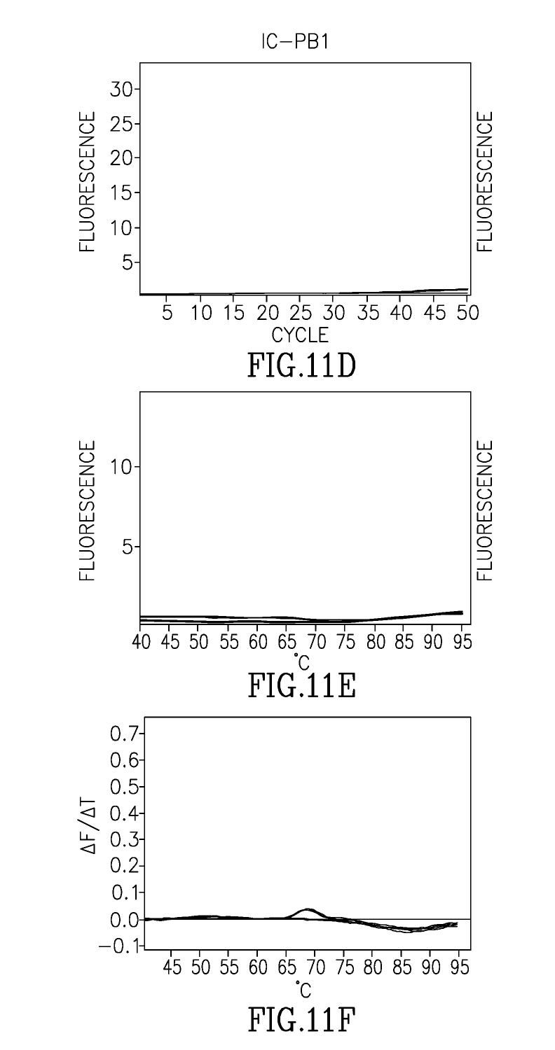

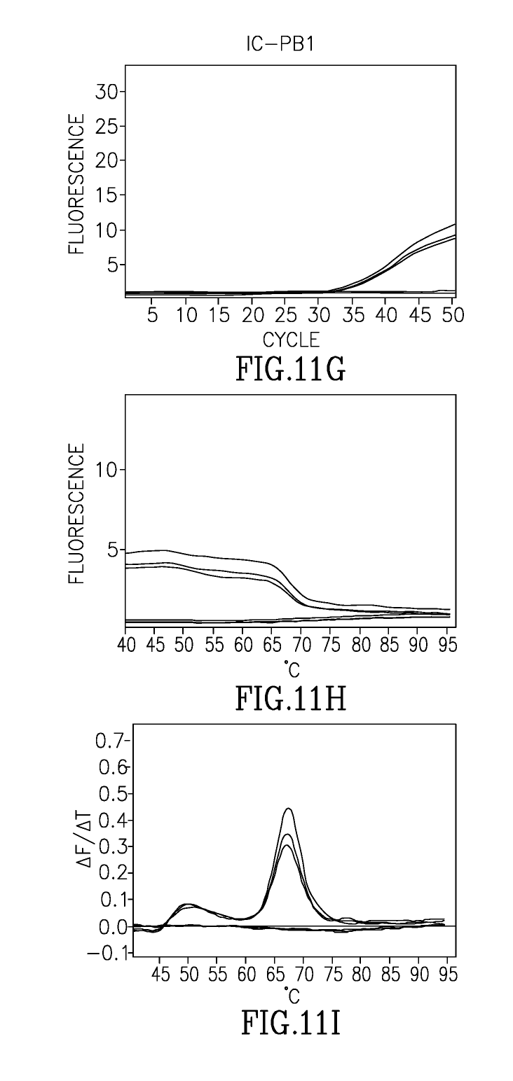

Two more IC probes were produced. Both probes were shorter than IC-PB2. IC-PB3 had similar ΔTM to IC-PB2, while IC-PB4 had a much reduced ΔTM. IC-PB4 exhibited a significant peak, with a low free probe background (FIG. 11), and is thus suitable for triplex amplification.

### EXAMPLE 9

### Serial Modification of the Tuf Probe

Probes and Primers

Forward and Reverse Tuf Primers

The forward and reverse tuf primers for are depicted in Table 7.

TABLE 7
tuf forward and reverse primers. The
ribonucleotide residue in the se-
quences is preceded with an “r”.

| SEQ | | |
|---|---|---|
| NO. | Name | Sequence |
| 25 | Tuf-F | GTGTTGAACGTGGTCAAATCAArAGTTG |
| 26 | Tuf-R | ATTGAACCAGGAGCAGCTAATrACTTG |

Probes for Tuf

The tuf probes are depicted in Table 8. Each probe was labeled with Cal Fluor® Red and BHQ2 on its 5′ and 3′ ends, respectively. Capital letters signify the hybridization region with the PCR product.

TABLE 8
tuf probes.

| SEQ | | | | |
|---|---|---|---|---|
| ID | Name | Sequence | Length | ΔTM |
| 43 | Tuf- | cgccagCCGTAAATTATTAGACTACGC | 38 | 10.5 |
| | PB1 | TGAAGCTGGcg | | |
| 44 | Tuf- | ccaccAGACTACGCTGAAGCTGGTGg | 26 | 13.5 |
| | PB2 | | | |
| 45 | Tuf- | caccAGACTACGCTGAAGCTGGTG | 24 | 7 |
| | PB3 | | | |

Results

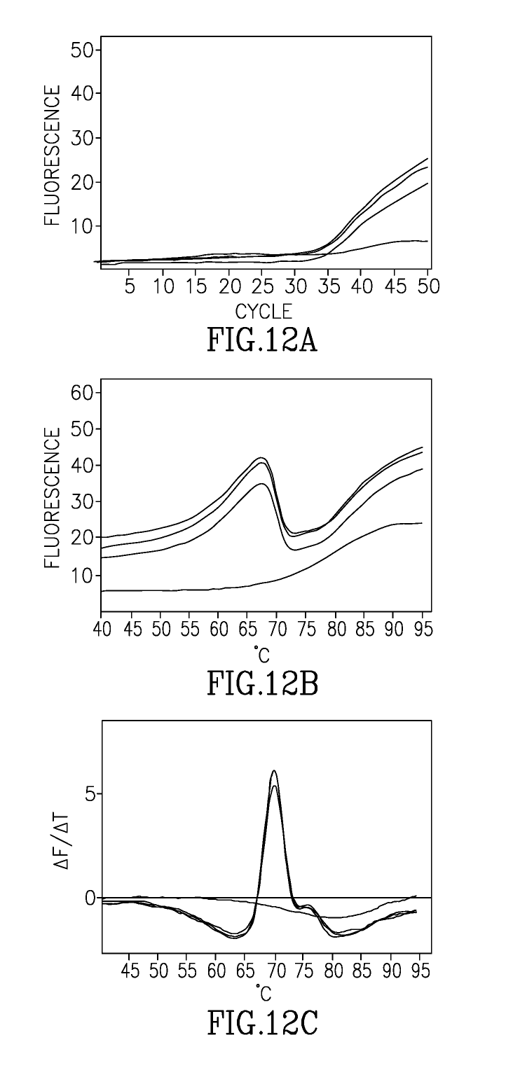

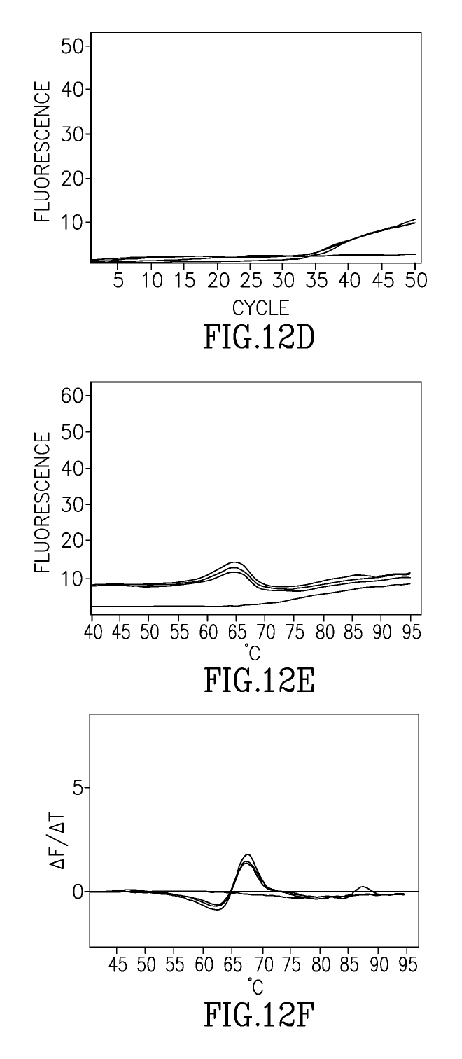

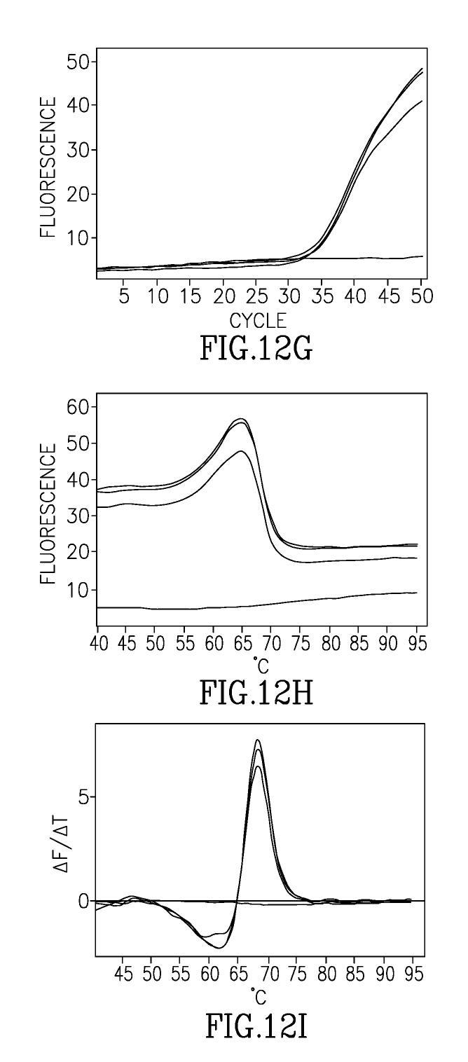

The first probe, tuf-PB1, produced a clearly detectable peak, but it was not believed to be suitable for triplex amplification, due to its high free probe background. The second probe, tuf-PB2, was shorter and had a lower free probe background. But the melting peak was significantly smaller. This problem was addressed by lowering the delta TM in the third version, tuf-PB3, which had the highest hybrid peak signal and the lowest free probe background (FIG. 12).

### EXAMPLE 10

### Successful Orange Channel Triplex Detection in the Context of the 5-Channel GN Multiplex Reaction

Probes and Primers (Examples 10-11)

Forward and Reverse Primers of the Gram-Negative Assay Tube

The forward and primers of the GN assay tube, other than those already mentioned (16S-GN, vim, NDM, and IC [the IC primers (and probe) are the same as for the GP tube]) and those omitted (IMP) are depicted in Table 9. The targets amplified are OprI (SEQ ID NOs: 21-22); SHV (SEQ ID NOs: 89-90); CTXM-14 (SEQ ID NOs: 23-24); CTXM-15 (SEQ ID NOs: 25-26); KPC (SEQ ID NOs: 27-28); GES (SEQ ID NOs: 29-30); OXA-48 (SEQ ID NOs: 31-32); and rpoB (SEQ ID NOs: 33-34).

TABLE 9
Additional GN tube forward and reverse primers.
The ribonucleotide residue in the sequence is
preceded with an “r”.

| SEQ | | |
|---|---|---|
| NO. | Name | Sequence |
| 21 | OprI_30917-F | TGA ACA ACG TTC TGA AAT TCT CTG CTrC TGG |
| | | C |
| 22 | OprI_30917-R | CTT GCG GCT GGC TTT TTC rCAG CA |
| 89 | SHV-F | CTG CTG ACC AGC CAG CGT rCTG AG |
| 90 | SHV-R | GCT CTG CTT TGT TAT TCG GGC rCAA GC |
| 23 | CTXM-14-F | GAT GAA CGC TTT CCA ATG TGC AGT rACC AG |
| 24 | CTXM-14-R | TCT GCC AGC GTC ATT GTG CCrG TTG A |
| 25 | CTXM-15-F | GGG CGC AGC TGG TGA CAT GrGA TGA |
| 26 | CTXM-15-R | CGC GAC GGC TTT CTG CCT TArG GTT G |
| 27 | KPC-F | CCA TTC GCT AAA CTC GAA CAG GArC TTT G |
| 28 | KPC-R | AGA AAG CCC TTG AAT GAG CTG rCAC AG |
| 29 | GES-F | CGAC ATT GGT TTT TTT AAA GCC CAG rGAG AG |
| 30 | GES-R | TGA GTT GTG TAA TAA CTT GAC CGA CrAG AGG |
| 31 | OXA48-F | GCG TAG TTG TGC TCT GGA ATG rAGA AT |
| 32 | OXA48-R | GTG TTC ATC CTT AAC CAC GCC CAA rATC GA |
| 33 | rpoB-F | GGTGGTCAGCGTTTCGGTGAGrATGGA |
| 34 | rpoB-R | TAGTCACCATTTTTTAGTTCAATGTTGrATACC |

Probes of the Gram-Negative Assay Tube

The probes of the GN assay tube, other than those already mentioned (16S-GN, vim, NDM, and IC) and those omitted (IMP) are depicted in Table 10. The probes recognize the excess strands of the following amplicons: OprI (SEQ ID NO: 35); SHV (SEQ ID NO: 94); CTXM-14 (SEQ ID NO: 36); CTXM-15 (SEQ ID NO: 37); KPC (SEQ ID NO: 38); GES (SEQ ID NO: 39); OXA-48 (SEQ ID NO: 40); rpoB (SEQ ID NO: 41); and 16S-GP (SEQ ID NO: 42)

Each probe was labeled with the indicated fluorophore. Capital letters signify the hybridization region with the PCR product.

TABLE 10
Additional GN Tube Probes.

| SEQ | | | |
|---|---|---|---|
| ID | Name | Sequence | Fluorophore |
| 35 | OprI_30917- | cgGGC TAC CGG TTG CAG CAG Cccg | FAM |
| | PB | | |
| 94 | SHV-PB | acctagCGATAAGACCGGAGCTAGgt | HEX |
| 36 | CTXM-14- | cggcaTC GAG ATC AAG CCT GCC G | HEX |
| | PB | | |
| 37 | CTXM-15- | ccccaGACT GCC TGC TTC CTG GGg | HEX |
| | PB | | |
| 38 | KPC-PB | ccggcTACAGTTGCGCCTGAGCCGG | Cal Fluor ® |
| | | | Red 610 |
| 39 | GES PB | ctccgTTCG TCA CGT TCT ACG Gag | Cal Fluor ® |
| | | | Red 610 |
| 40 | OXA48-PB | ccgcatGG AAT TTT AAA GGT AGA | CAL Fluor ® |
| | | TGC GG | Red 610 |
| 41 | rpoB-PB | ctcggTTGACCAAAGAGATCCGag | Quasar ® 670 |
| 42 | 16S-GP-PB | CGCGCTgacaaccatgcaccacctgAGCGCG | Quasar ® 670 |

Results

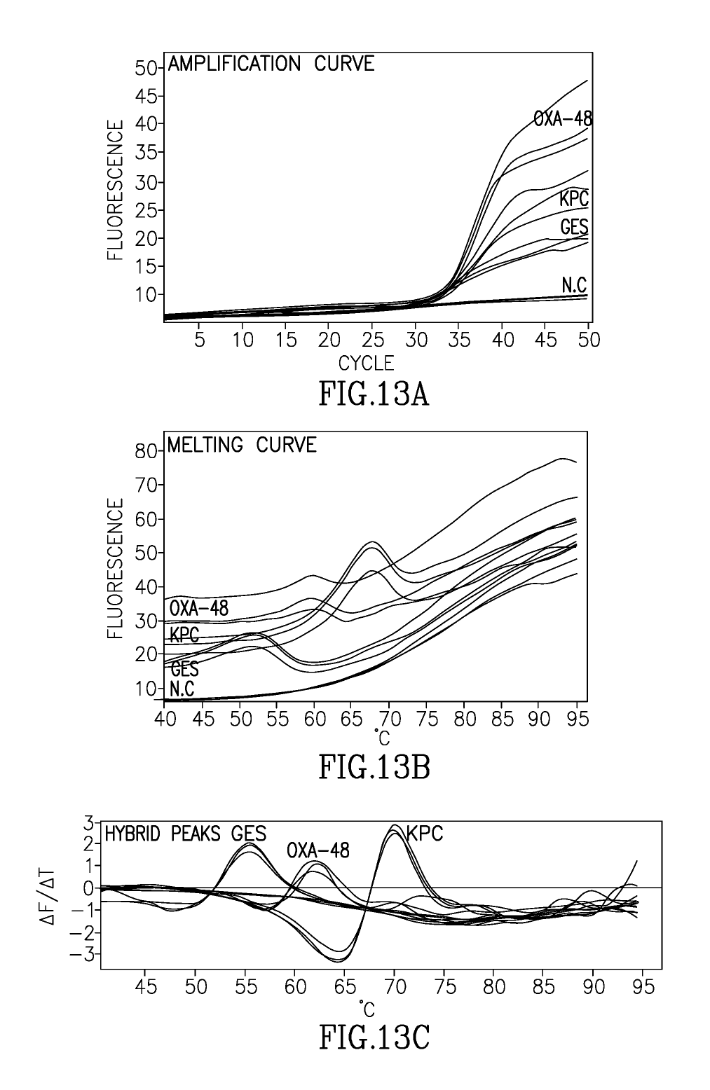

Next, amplifications of GES, OXA-48, and KPC, the orange channel targets, were performed, in three separate amplifications in the presence of almost the entire set of primers for the 5-channel GN multiplex reaction (all except the IMP primers and probes, which were still being finalized). Three clear and distinguishable peaks were observed in this channel (FIG. 13).

### EXAMPLE 11

### Successful Crimson Channel Triplex Detection in the Context of the 5-Channel GN Multiplex Reaction

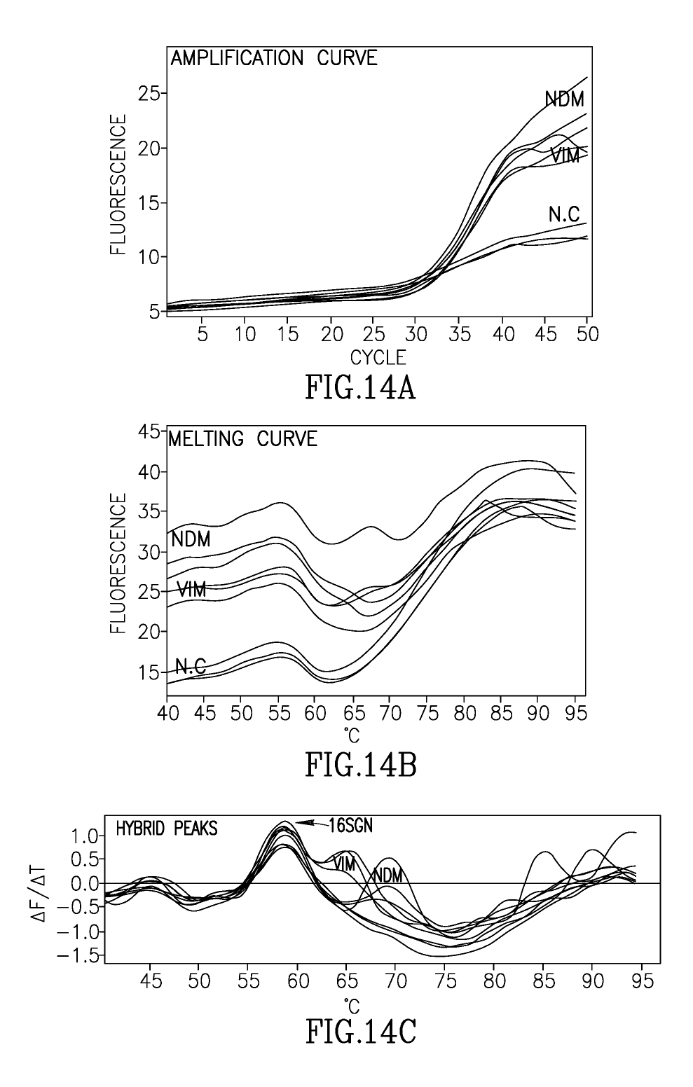

Next, amplifications of vim, NDM, and 16S-GN, the crimson channel targets, were performed, in three separate amplifications in the presence of the multi-channel set of primers described in the previous Example. Three clear and distinguishable peaks were observed in this channel (FIG. 14).

### EXAMPLE 12

### Utilization of Relatively GC-Poor Regions for Primer Binding Improves Amplification of a GC-Rich KPC Amplicon

TABLE 16
Forward and reverse primers and probes for amplification of a GC-rich
KPC amplicon. The ribonucleotide residues in the primer sequences are
preceded with an “r”. BC and AC refer to before and after cleavage,
respectively. T_{M} refers to the hybrid T_{M}. Capital letters in the
probe sequences signify the hybridization region.
PRIMERS

| Name/SEQ | | Length | GC % | TM | GC % | T_{M} |
|---|---|---|---|---|---|---|
| NO. | Sequence | (bp) | BC | BC | AC | AC |
| KPC-F2/ | CCATTCGCTAAACTCGAACAG | 28 | 46.4 | 70.2 | 47.8 | 67.5 |
| 95 | GArCTTTG | | | | | |
| KPC-R2/ | AGAAAGCCCTTGAATGAG | 26 | 50 | 68.9 | 47.6 | 62.9 |
| 96 | CTGrCACAG | | | | | |
| NDM-F2/ | TCGACAACGCATTGGCA | 24 | 50 | 67.6 | 47.4 | 62.4 |
| 97 | TArAGTCG | | | | | |
| NDM-R2/ | AACTGGATCAAGCAGGA | 26 | 46.2 | 69.7 | 47.6 | 65.1 |
| 98 | GATCrAACCT | | | | | |

PROBES

| Name/SEQ | | Length | | |
|---|---|---|---|---|
| NO. | Sequence | (bp) | GC % | T_{M} |
| KPC-PB/ | ccggcTACAGTTGCGCCTGAG | 25 | 72 | 72.1 |
| 99 | CCGG, labeled w/QS705/BHQ2 | | | |
| NDM-PB3/ | CGCCAGTccatcttgtcctgatgcgcgtg | 44 | 61.4 | 77.5 |
| 100 | agtcaccaACTGGCG | | | |

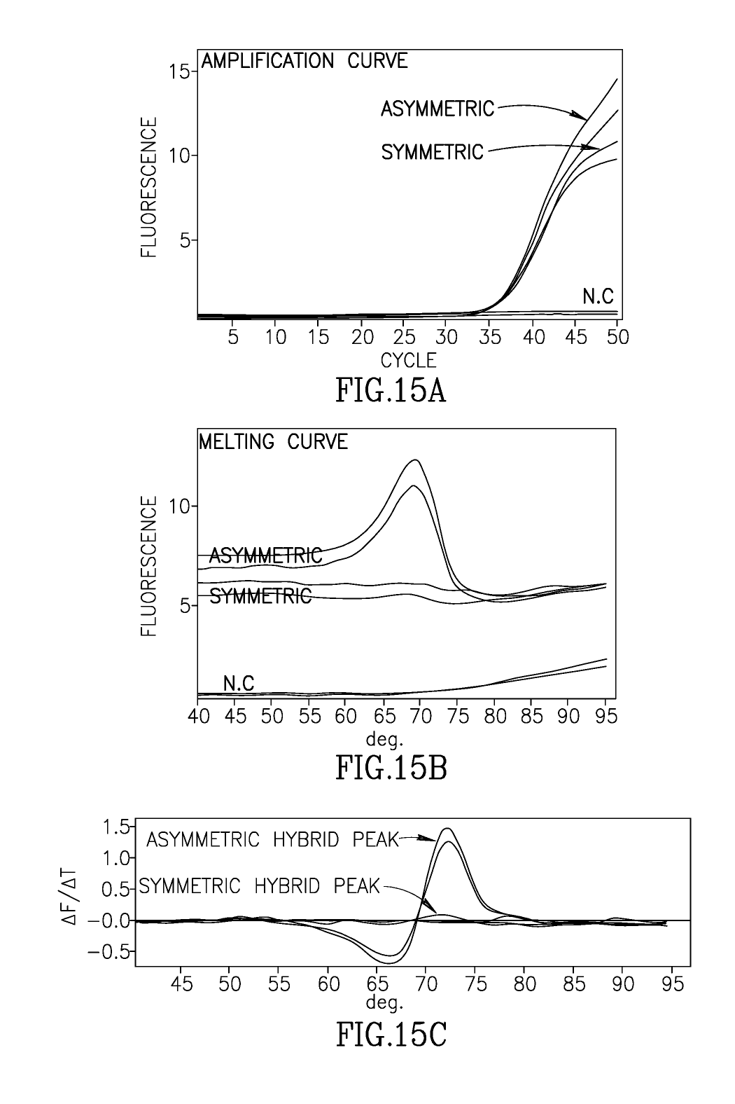

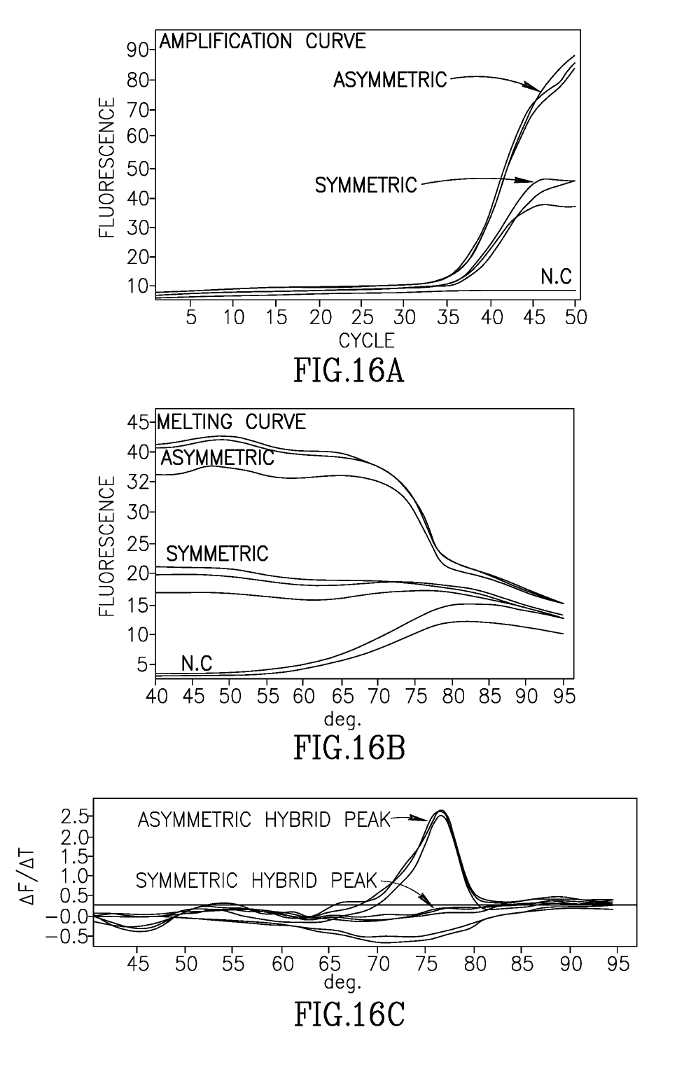

Asymmetric amplification of a GC-rich region (56.5% GC content) from KPC was attempted, using the primers KPC-F2 and KPC-R2 at 1 and 0.1 μM concentration, respectively. The predicted T_{M} for the amplicon was 88.9 C. The excess primer was designed to target a relatively GC-poor region of the amplicon (46.4% GC content). The amplification worked efficiently, as evidenced by the strong signal that was not seen with the symmetric PCR, as expected with a probe that binds only single-stranded product. The successful amplification worked despite the ΔT_{M} between the amplicon and a hybrid of the pre-cleavage excess primer and its target being 18.7° C., which is generally considered high (FIG. 15). Similar results were obtained with the NDM amplicon, in this case with NDM-PB3, a long probe of 44 bases—in that case, the reverse primer was the excess primer (FIG. 16). The amplicon sequences were as follows:

KPC:
(SEQ ID NO: 101)
ccattcgctaaactcgaacaggactttggcggctccatcggtgtgtacgc
gatggataccggctcaggcgcaactgtaagttaccgcgctgaggagcgct
tcccactgtgcagctcattcaagggctttct.
NDM:
(SEQ ID NO: 102)
aactggatcaagcaggagatcaacctgccggtcgcgctggcggtggtgac
tcacgcgcatcaggacaagatgggcggtatggacgcgctgcatgcggcgg
ggattgcgacttatgccaatgcgttgtcga.

### REFERENCES

Abdeldaim G M, Stralin K, Olcen P, Blomberg J, Herrmann B. Toward a quantitative DNA-based definition of pneumococcal pneumonia: a comparison of Streptococcus pneumoniae target genes, with special reference to the Spn9802 fragment. Diagn Microbiol Infect Dis 2008;60:143-50.

Daniels, R. Surviving the first hours in sepsis: getting the basics right (an intensivist's perspective), 2011, Journal Antimicrob. Chemother. 66 (suppl 2): ii11-23.

Dobosy J R, Rose S D, Beltz K R, Rupp S M, Powers K M, Behlke M A, Walder J A. RNase H-dependent PCR (rhPCR): improved specificity and single nucleotide polymorphism detection using blocked cleavable primers. BMC Biotechnol. 2011. 11:80.

Gentile N L, Dillier A M, Williams G V, Ackers J, Reis A H Jr, Rice L M, Wangh L J, Czajka J W, Kost G J. Verification of monoplex and multiplex linear-after-the-exponential PCR gene-specific sepsis assays using clinical isolates. J Appl Microbiol. 2013 February; 114(2):586-94.

Hall, M J Williams, S M, DeFrances, C J, and Golosinskiy, A. Inpatient Care for Septicemia or Sepsis: A Challenge for Patients and Hospitals, 2011, NCHS Data Brief, No. 62.

Tao Luo, Lili Jiang, Weiming Sun, G. Fu, Jian Mei, and Qian Gao, Multiplex Real-Time PCR Melting Curve Assay To Detect Drug-Resistant Mutations of Mycobacterium tuberculosis. J Clin Microbiol. 2011 September; 49(9): 3132-3138.

Rice L M, Reis A H Jr, Ronish B, Carver-Brown R K, Czajka J W, Gentile N, Kost G, Wangh L J. Design of a single-tube, endpoint, linear-after-the-exponential-PCR assay for 17 pathogens associated with sepsis. J Appl Microbiol. 2013 February; 114(2):457-69

1-82. (canceled)

83. A method for detecting the presence of a target polynucleotide in a test sample, comprising the steps of:
(a) thermocycling a reaction mixture, while periodically measuring fluorescence at 4 or more different channels, the reaction mixture comprising:
the test sample;
6 or more primer sets, wherein at least the majority of the primer sets are asymmetric; and
6 or more probes, which fluoresce in 4 or more different channels,
wherein each of said probes binds to a polynucleotide selected from the group consisting of: a PCR product of a target amplified by one or more of the primer sets; and a control polynucleotide, whereupon fluorescence of the probe is activated;
wherein in at least one of the channels, a plurality of different target-probe fluorescence signatures are discriminable; and
wherein forward and reverse primers of at least the majority of the primer sets are hot-start primers;
(b) subjecting the product of step (a) to a controlled heating or controlled cooling, while periodically measuring fluorescence at each of the channels; and
(c) for each channel in which a plurality of different target-probe fluorescence signatures are discriminable, identifying the fluorescence signature that is present, thereby detecting the presence of the target polynucleotide.

84. The method of claim 83, wherein in the 4 or more different channels, a plurality of different target-probe fluorescence signatures are discriminable.

85. The method of claim 83, wherein the reaction mixture is present in a single PCR reaction tube, split into two PCR reaction tubes, in a single well of a reaction plate or split into two wells of a reaction plate, wherein the targets comprise at least one pathogen marker polynucleotide and at least one polynucleotide associated with an antibiotic resistance in said pathogen, and
wherein for each channel in which a signal is present and a plurality of different target-probe fluorescence signatures are discriminable, the signature indicates which target has been amplified.

86. The method of claim 83, wherein for each channel in which most or all of the probes that fluoresce in the channel has a length of between 19-26 nucleotides inclusive, and at least one probe that fluoresces in the channel is a shared-stem probe.

87. The method of claim 83, wherein, for most of the probes, the internal melting temperature (T_{M}) of the stem of the probe is 6-13° C. inclusive higher than the T_{M} of a hybrid of the probe with the target sequence that is desired to be detected; or, if more than one target sequence is desired to be detected, the T_{M} of the probe is 6-13° C. inclusive higher than the T_{M} of a hybrid of the probe with each target sequence.

88. The method of claim 83, wherein, for each instance in which a probe also hybridizes to a known target sequence that is not desired to be detected on the channel, the internal T_{M} of the probe is at least 17° C. higher than the T_{M} of a hybrid of the probe with the known target sequence that is not desired to be detected on the channel.

89. The method of claim 83, wherein the primer sets amplify a set of targets comprising a Staphylococcus aureus (SA) marker polynucleotide; a polynucleotide selected from a non-SA Staphylococcus marker polynucleotide and a general Staphylococcus marker polynucleotide; an Enterococcus marker polynucleotide, a Streptococcus pneumoniae marker polynucleotide, a nucleotide sequence associated with vancomycin resistance, and a nucleotide sequence associated with methicillin resistance; and
wherein the probes collectively fluoresce in 4-7 different channels.

90. The method of claim 89, wherein the group of probes includes more than one probe that detects a nucleotide sequence associated with vancomycin or methicillin resistance, and the more than one probe fluoresce in the same channel.

91. The method of claim 89, wherein the targets further comprise one or more fungus marker polynucleotides.

92. The method of claim 83, wherein the reaction mixture is present in a single PCR reaction tube, split into two PCR reaction tubes, in a single well of a reaction plate or split into two wells of a reaction plate, wherein the primer sets amplify a set of targets comprising a gram-negative bacteria marker polynucleotide, a metallo-β-lactamase nucleotide sequence, a serine-β-lactamase nucleotide sequence, and a nucleotide sequence of an extended-spectrum- or broad-spectrum β-lactamase; and
wherein the probes collectively fluoresce in 4-7 different channels.

93. The method of claim 92, wherein the metallo-β-lactamase is at least one selected from the group consisting of IMP-1, IMP-2, IMP-3, IMP-4, vim, NDM-1, NDM-2, NDM-3, NDM-4, NDM-5, NDM-6, and NDM-7, and
wherein the group of probes includes at least 2 probes that detect a metallo-β-lactamase, and which fluoresce in 1-2 channels.

94. The method of claim 92, wherein the serine-β-lactamase is at least one selected from the group consisting of KPC-2, KPC-3, KPC-4, KPC-5, KPC-6, KPC-7, KPC-8, KPC-9, KPC-10, KPC-11, GES, and OXA-48, and
wherein the group of probes includes at least 2 probes that detect a serine-β-lactamase, and which fluoresce in 1-2 channels.

95. The method of claim 92, wherein the extended-spectrum- or broad-spectrum-β-lactamase is at least one selected from the group consisting 2be or 2br variant of SHV β-lactamase, CTXM-14, and CTXM-15, and
wherein the group of probes includes at least 2 probes that detect a 2be or 2br variant of SHV β-lactamase, and which fluoresce in 1-2 channels.

96. The method of claim 89, wherein the group of primer sets consists of 10 or more primer sets inclusive.

97. A method of confirming and determining a causative pathogen of a suspected case of sepsis, comprising the method of claim 89, and further comprising
using a logic matrix to identify the pathogenic agents and antibiotic-resistance polynucleotides present in said test sample.

98. A method of confirming and determining a causative pathogen of a suspected case of sepsis, comprising the method of claim 91, and further comprising
using a logic matrix to identify the pathogenic agents and antibiotic-resistance polynucleotides present in said test sample.

99. A method of confirming and determining a causative pathogen of a suspected case of sepsis, comprising the method of claim 92, and further comprising
using a logic matrix to identify the pathogenic agents and antibiotic-resistance polynucleotides present in said test sample.

100. A method of detecting the presence of a polynucleotide in a test sample, the method comprising thermocycling a reaction mixture, while periodically measuring fluorescence at each of 4 or more different said channels,
wherein said reaction mixture comprises: a test sample;
at least one primer set; and
6 or more probes, which fluoresce in the 4 or more different channels, wherein, for at least one primer set in the reaction mixture:
the primer set is asymmetric;
forward and reverse primers of the primer set are hot-start primers that contain an inactivating chemical modification that is reversed by the action of an activating enzyme, where the hot-start primers become a substrate for the activating enzyme when the hot-start primers are hybridized to a complementary sequence at elevated temperatures;
the melting temperature of the amplicon produced by the primer set exceeds the initial, concentration-adjusted melting temperature of a hybrid of the pre-cleavage excess primer and its target polynucleotide by more than 13° C.;
the initial, concentration-adjusted melting temperature of a hybrid of the pre-cleavage excess primer and its target polynucleotide is not more than 17° C. higher than the annealing temperature of the thermocycling; and
the initial, concentration-adjusted melting temperature of a hybrid of the post-cleavage excess primer and its target polynucleotide is at least 9° C. higher than the annealing temperature of said thermocycling;
wherein each of the probes binds to a polynucleotide selected from the group consisting of: a PCR product of a target amplified by one or more of the primer sets; and
a control polynucleotide, whereupon fluorescence of the probe is activated; and
wherein in at least one of the channels, a plurality of different target-probe fluorescence signatures are discriminable.

101. A method of detecting the presence of a polynucleotide in a test sample, comprising thermocycling a reaction mixture, while periodically measuring fluorescence at each of 4 or more different channels, wherein the reaction mixture comprises
a test sample
at least one primer set; and
6 or more probes, which fluoresce in the 4 or more different channels,
wherein, for at least one primer set in the reaction mixture:
the primer set is asymmetric;
forward and reverse primers of the primer set contain an inactivating chemical modification that is reversed by the action of an activating enzyme, where the primers become a substrate for the activating enzyme when the primers are hybridized to a complementary sequence at elevated temperatures;
the melting temperature of the amplicon produced by extension of the primer set exceeds the initial, concentration-adjusted melting temperature of a hybrid of the pre-cleavage excess primer and its target polynucleotide by more than 13° C.;
the GC content of said amplicon is at least 50%; and
the GC content of the region bound by the excess primer of the primer set is at least 2% lower than said GC content of the amplicon
wherein each of the probes binds to a polynucleotide selected from the group consisting of: a PCR product of a target amplified by one or more of said primer sets; and
a control polynucleotide, whereupon fluorescence of the probe is activated; and
where, in at least one of said channels, a plurality of different target-probe fluorescence signatures are discriminable.
